(19) United States
(12) Patent Application Publication
(10) Pub. No.: US 2011/0166028 A1
(43) Pub. Date: Jul. 7, 2011

(54) METHODS FOR PREDICTING TREATMENT RESPONSE BASED ON THE EXPRESSION PROFILES OF BIOMARKER GENES IN NOTCH MEDIATED CANCERS

(76) Inventors: Donald Bergstrom, Warrington, PA (US); Xudong Dai, Kirkland, WA (US); James Hardwick, Princeton Junction, NJ (US); Cole Liberator, Brookline, MA (US); A. Thomas Look, North Reading, MA (US); Jennifer O'Neil, Dedham, MA (US); Sudhir Rao, Somerville, MA (US); Peter Strack, Reading, MA (US); Christopher Winter, Needham, MA (US); Theresa Zhang, Wellesley, MA (US)

(21) Appl. No.: 12/674,981

(22) PCT Filed: Aug. 22, 2008
(86) PCT No.: PCT/US08/10006

§ 371 Date: Feb. 24, 2010

Related U.S. Application Data
(60) Provisional application No. 60/966,450, filed on Aug. 28, 2007.

(51) Int. Cl. C40B 30/00; C12Q 1/68; G01N 33/53

(52) U.S. Cl. 506/7; 435/6.12; 435/7.1

(57) ABSTRACT

The invention relates to the identification and use of gene expression profiles with clinical relevance to the treatment of cellular proliferative disorders, especially those mediated by aberrant Notch signaling using a Notch signaling inhibitor. In particular, the invention provides the identities of genes, whose individual or cumulative expression patterns may be useful in various assays. The gene expression profiles, whether embodied in nucleic acid expression, protein expression, or other expression formats, may be used to select subjects afflicted with a Notch mediated cancer who will likely respond to treatment with a gamma-secretase inhibitor or another Notch inhibiting agent. The same markers may be used in the classification of patients being treated with other Notch inhibitors. The methods may further comprise providing diagnostic, prognostic, or predictive information based on the classifying step. The methods may further comprise selecting a treatment based on the classifying step.

### BACKGROUND OF THE INVENTION

(1) Field of the Invention

The present invention relates generally to the identification of novel biomarkers and their use including prognostic assay for parameters which are indicative of a condition or event associated with the aberrant Notch signaling. The expression patterns of individual or collective biomarkers detailed herein are useful for risk assessment, early detection, establishing prognosis, and evaluation of intervention. More particularly, the present invention provides an assay to detect parameters associated with a Notch mediated cellular proliferative disorders, especially cancer. The identification of a specific gene expression profile or encoded protein expression parameters or more particularly a pattern of parameters enables the prognosis of patients sensitive to treatment with a Notch inhibitor or the identification of a patient at risk of failing treatment with a Notch inhibitor. The biomarker expression parameters may also be useful in stratifying patients for a clinical trial as well as establishing a therapeutically effective dose of a Notch inhibitor.

In the main, the invention relates to the identification and use of gene expression profiles, or patterns, with clinical relevance to the treatment of cellular proliferative disorders, especially those mediated by aberrant Notch signaling using a Notch signaling inhibitor.

In particular, the invention provides the identities of various genes, such as HES1, DTX1. MYC, p19, etc, whose expression pattern is correlated with patient survival and treatment outcome especially in patients treated with a Notch inhibitor, such as a gamma secretase inhibitor or another “Notch” inhibiting (“iNotch”) agent. The gene expression profiles, whether embodied in nucleic acid expression, protein expression, or other expression formats, may be used to select subjects afflicted with a Notch mediated cancer who will likely respond positively to treatment with the gamma-secretase inhibitor or another iNotch agent against Notch mediated cancers as well as those who will likely be non-responsive and thus candidates for other treatments.

(2) Description of Related Art

Cancer is the end point of the accumulation of genetic mutations caused, in part, by inherited, viral or environmental insults. The increased number of cancer cases reported in the United States, and, indeed, around the world, is a major concern. Currently there are only a handful of treatments available for specific types of cancer, and these provide no guarantee of success. In order to be most effective, these treatments require not only an early detection of the malignancy, but a reliable assessment of the severity of the malignancy.

Several lines of evidence indicate that tumorigenesis in humans is a multistep process and that these steps reflect genetic alterations that drive the progressive transformation of normal human cells into highly malignant derivatives. Towards this end, there is mounting evidence suggesting that deregulated expression and/or activity of wild-type Notch receptors occurs frequently in human malignancies and that constitutively active Notch receptors have transforming activity. Indeed, the importance of Notch receptors and components of this cascade during development has indicated that this pathway is involved in a wide range of events, intimately involved with key cellular processes such as differentiation, proliferation and apoptosis. Support for this conclusion is apparent from studies of Drosophila, C. Elegans, zebrafish and mammals which have demonstrated that the Notch pathway is an evolutionarily conserved signaling mechanism that functions to modulate numerous cell-fate decisions. Indeed, Notch signaling has been shown to directly affect numerous cellular programs, including proliferation, differentiation and apoptosis and these events are highly dependent on signal strength and cellular context. Artavanis-Tsakonas et al., Science 268:225-232 (1995); Kadesch, T., Exp. Cell. Res. 260:1-8 (2000). Depending on the cellular context, Notch signaling may both inhibit and induce differentiation, induce proliferation, and promote cell survival—Artavanis-Tsakonas et al., 1995, supra; Lewis, 1998; Weinmaster, J. Virol., 71:1938-45 (1997). In fact, Notch signaling appears to influence many different types of cell-fate decisions by providing inhibitory, inductive or proliferative signals depending on the environmental context. Reviewed in Artavanis-Tsakonas et al., 1995, supra; Greenwald, 1998; Robey, Curr Opin Genet Dev., 7:551-7 (1997); Vervoort et al., Curr Opin Neurobiol., 7:21-28 (1997). This pleiotropic function suggests that Notch modulates multiple signaling pathways in a spatio-temporal manner.

Consistent with Notch regulating cell-fate decisions, the four mammalian Notch genes encode large, multidomain proteins that consist of a single transmembrane domain and large extracellular and intracellular domains. The Notch receptor family includes Notch in Drosophila, LIN-12 and GLP-1 in C. elegans, and mNotch1 and mNotch2 in mouse, among others. Artavanis-Tsakonas et al. (1995) Science 268:225-232. Five mammalian ligands have been described so far, Delta-like-1, Delta-like-3 and Delta-like-4 (DLL1, DLL3 and DLL4) and Jagged 1 and Jagged2 (JAG1 and JAG2).

The current model for Notch signaling suggests that it is elicited by receptor-ligand interaction between two neighboring cells. Receptor-ligand interaction leads to two successive proteolytic cleavages of Notch, resulting in the release of the intracellular domain of the receptor (icNotch). This part of the receptor translocates to the nucleus where it converts the transcription factor CBF1/Su(H)/LAG1 (CSL) from a repressor to a transcriptional activator. As of yet, only a limited number of target genes have been defined, though members of the Hairy-Enhancer of split (HES) and Hes-related protein (HERP/HEY) families are important in many tissues. They belong to the basic helix-loop-helix family of transcription factors and act as transcriptional repressors, suppressing expression of cell type specific target genes. Li, X., and Greenwald, I. Proc. Natl. Acad. Sci. USA 95:7109-7114 (1998); Thinakaran et al., Neuron 17:181-190 (1996); Podlisny et al., Neurobiol. Dis. 3:325-337 (1997); Capell et al., J. Biol. Chem. 273:3205-3211 (1998). Thus, Notch appears to undergo proteolytic events that resemble those involved in cleavage of APP, i.e., sequential hydrolysis by β (beta) and γ (gamma) secretases. Jarrault et al., Nature, 377: 355-358 (1995).

Over the years much has been learned about the regulation and function of Notch signaling during development, but the link to tumorigenesis was for some time restricted to malignancies wherein structural DNA rearrangements affecting the Notch receptor were implicated. Now, a growing body of evidence suggests that augmented or abnormally-prolonged Notch signaling is involved in tumorigenesis. Callahan and Egan, J. Mammary Gland Biol. Neoplasia (2004), 9, 145-163; Collins et al, Semin. Cancer Biol. (2004), 14, 357-64; Axelson, ibid. (2004), 14, 317-319; Zweidler-McKay and Pear, ibid (2004), 14, 329-340; and Weng et al, Mol. Cell. Biol. (2003), 23, 655-664).

The first example of Notch and its link to tumorigenesis was described in a subset of T-cell acute lymphoblastic leukemia (T-ALL) carrying the (7;9) (q34;q34.3) translocation, as reviewed in detail by Zweidler-McKay and Pear, supra. In these tumors Notch1 was found to be fused to the T-cell receptor β (TCRβ) locus, leading to constitutive expression of the intracellular domain of Notch1, and subsequent facsimile experiments in mice have confirmed the oncogenic effect of Notch activation in T-cells. Significantly, although the t(7;9) translocation is only found in a limited subset of T-ALL, subsequent studies have shown that almost all T-ALL express high levels of Notch1 or Notch3. Notch signaling and cancer: emerging complexity, Seminars in cancer Biology, 14:317-319 (2004).

Modified Notch1 signaling has also been implicated in lymphoblastic leukemia/lymphomas, mammary gland tumors, lung cancer, colon cancer, neuroblastomas, skin cancer, cervical cancer, epithelial tumors and prostate cancer. See Allenspach et al., Cancer Biology and Therapy, 1:5, 466-476, (2002). Activating mutations in Notch1 are also implicated in human T Cell Acute Lymphoblastic Leukemia (T-ALL), Weng, et al., Science, 306:269-271 (2004).

Taken together, the data suggest that perturbations in the regulation of Notch signaling are responsible for malignant transformation. Maillard et al., Immunity 19:781-791 (2003); Radtke et al., Nat. Rev. Cancer. 3:756-767 (2003) and tumor suppression function. See Nicolas et al., Nat. Genet. 33:416-421 (2003); Radtke et al., Nat. Rev. Cancer. 3:756-767 (2003).

In view of the involvement in tumorigenesis, there has been much interest in inhibition of Notch signaling as a method of treating malignancies. Various types of intervention in the signaling process have been considered, such as inhibiting expression of the Notch protein, blockade of the receptor to prevent ligand binding, and inhibition of the intra-membrane proteolysis.

Towards this end, a convergence of evidence indicates that the gamma secretase complex, comprised of the presenilin subunits, in addition to APP processing leading to .beta.-amyloid synthesis, mediates the intra-membrane cleavage of other type I transmembrane proteins (reviewed in Fortini, M. E. (2002). “Gamma-secretase-mediated proteolysis in cell-surface-receptor signaling” Nat Rev Mol Cell Biol 3(9): 673-84, see also Struhl, G. and A. Adachi (2000). “Requirements for presenilin-dependent cleavage of notch and other transmembrane proteins.” Mol Cell 6(3): 625-36.) Noteworthy among the known substrates of gamma-secretase is mammalian Notch 1. The Notch 1 protein is important for cell fate determination during development, and tissue homeostasis in the adult.

Support for this is evident from various studies. For example, disruption of Notch signaling via genetic knock-out (KO) results in embryonic lethal phenotype in mice. Swiatek, P. J., C. E. Lindsell, F. F. del Amo, G. Weinmaster and T. Gridley (1994). “Notch1 is essential for postimplantation development in mice.” Genes Dev 8(6): 707-19; Conlon, R. A., A. G. Reaume and J. Rossant (1995). “Notch1 is required for the coordinate segmentation of somites.” Development 121(5): 1533-45.) The Notch KO phenotype is very similar to the phenotype observed PS1 KO mice, and precisely reproduced by PS1/PS2 double KO mice (De Strooper et al., “Deficiency of presenilin-1 inhibits the normal cleavage of amyloid precursor protein.” Nature 391(6665): 387-90 (1998); Donoviel, D. B., A. K. Hadjantonakis, M. Ikeda, H. Zheng, P. S. Hyslop and A. Bernstein, “Mice lacking both presenilin genes exhibit early embryonic patterning defects.” Genes Dev. 13(21): 2801-10 (1999); Herreman, A., L. Serneels, W. Annaert, D. Collen, L. Schoonjans and B. De Strooper, “Total inactivation of gamma-secretase activity in presenilin-deficient embryonic stem cells.” Nat Cell Biol. 2(7): 461-2 (2000).

Cellular proliferative disorders such as cancer account for nearly one-quarter of deaths in the United States, exceeded only by heart diseases. The disease contributes to a major financial burden to the community and to individuals. A central paradigm in the care and treatment of patients presenting with cellular proliferative disorders mediated by Notch is to offer better risk assessment, screening, diagnosis, prognosis and selection and monitoring of therapy. Such cellular proliferative disorders are those affected by aberrant Notch signaling, particularly where Notch is over-expressed relative to normal. Methods for quantifying normal expression are well known.

In clinical practice, accurate diagnosis of various subtypes of cancer is important because treatment options, prognosis, and the likelihood of therapeutic response all vary broadly depending on the diagnosis. Accurate prognosis as well as a determination of treatment outcome could allow the oncologist to tailor the administration of therapy with patients having poorer prognoses being given the most aggressive treatment. Furthermore, accurate prediction of treatment outcome, favorable or poor prognosis would greatly impact clinical trials for new cancer therapies, because potential study patients could then be stratified according to prognosis. Trials could then be limited to patients having poor prognosis, in turn making it easier to discern if an experimental therapy is efficacious. In the early clinical development of anti-cancer agents, clinical trials are typically designed to evaluate the safety, tolerability, and pharmacokinetics, as well as to identify a suitable dose and schedule for further clinical evaluation. Scientists believe that the development of new validated biomarkers will lead to significant reductions in healthcare and drug development costs as well as provide a tool for achieving successful preventive intervention. Since early diagnosis and prognosis is the key to surviving cancer, identification of disease biomarkers or biomarkers predictive of response to treatment with a particular moiety or a class thereof has been an active research area. Increasingly, efforts are being expended towards discriminating patients sensitive to treatment with a Notch inhibitor from those resistant to such therapy.

Although conventional histological and clinical features have been correlated with prognosis, new prognostic and predictive markers are needed to accurately foretell a patient's response to drugs in the clinic. Such markers would facilitate the individualization of therapy for each patient. Thus, there is an on-going need towards identifying subjects afflicted with a Notch mediated cancer who will likely respond positively to treatment with an iNotch agent as well as those who will likely be non-responsive and thus candidates for other treatments. This would allow for the earlier identification of patients favored to respond positively to treatment with an iNotch agent but also towards the identification of non-responders, e.g., at-risk patients, which would help in the development of molecular-targeted interventions to prevent or delay neoplasia. Mindful that prognosis and prediction of response are necessary for the selection of neoadjuvant or adjuvant chemotherapy, it would be useful to be able to identify clinically relevant intermediate end points, which may predict not only the final outcome of a chemopreventive trial but also help identify high-risk patients. After all, avoiding ineffective therapies is as important as identifying effective ones.

As a consequence, a great deal of effort is being directed to using new technologies to find new classes of biomarkers, which is becoming one of the highly prized targets of cancer research. See Petricoin et al, Nature Reviews Drug Discovery, 1: 683-695 (2002); Sidransky, Nature Reviews Cancer, 2: 210-219 (2002). Recently, many studies have used gene expression profiling to analyze various cancers, and those studies have provided new diagnosis and prognosis information in the molecular level. See Zajchowski et al., —‘Identification of Gene Expression Profiled that Predict the Aggressive Behavior of Breast Cancer Cells,” Cancer Res. 61:5168 (2001); West et al, “Predicting the Clinical Status of Human Breast Cancer by Using Gene Expression Profiles,” Proc. Natl. Acad. Sc. U.S.A. 98:11462 (2001); van't Veer et al., “Gene Expression Profiling Predicts the Outcome of Breast Cancer,” Nature 415:530 (2002); Roberts et al., “Diagnosis and Prognosis of Breast Cancer Patients,” WO 02/103320; Sorlie et al, Proc. Natl. Acad. Sc U.S.A. 100:8418 (2003); Perou et al, Nature 406:747 (2000); Khan et al, Cancer Res 58, 5009 (1998); Golub et al, Science 286, 531 (1999); Alizadeh et al, Nature 403, 503 (2000). Methods for the identification of informative genesets for various cancers have also been described. See Roberts et al., “Diagnosis and Prognosis of Breast Cancer Patients,” WO 02/103320; Golub et al, U.S. Pat. No. 6,647,341.

Genesets have been identified that are informative for—differentiating individuals having, or suspected of having, breast cancer based on estrogen receptor (ER) status, or BRCA1 mutation vs. sporadic (i.e., other than BRCA1-type) mutational status. See Roberts et al, WO 02/103320; van't Veer et al., Nature 415:530 (2001). Genesets have also been identified that enable the classification of sporadic tumor-type individuals as those who will likely have no metastases within five years of initial diagnosis (i.e., individuals with a good prognosis) or those who will likely have a metastasis within five years of initial diagnosis (i.e., those having a poor prognosis). Roberts, supra; van't Veer, supra. Roberts et al. WO 02/103320 describes a 70-gene set, useful for the prognosis of breast cancer, which outperformed clinical measures of prognosis, and which showed good potential in selecting good outcome patients, thereby avoiding over-treatment, van de Vijver et al, N. Engl. J. Med. 347:1999 (2002).

Overall, prognostic biomarkers will find use not only in diagnosis but also predict response to therapy, identify potential candidates who may best be suited for a particular chemopreventive intervention, aid in the rational design of future intervention therapy. The study of biomarkers that can possibly predict how a person's disease may progress or respond to treatment, falls under the category of chemoprevention. Biomarkers used to measure a response to an intervention are called surrogate endpoint biomarkers or SEBs (Kelloff et al., Cancer Epidemiology, Biomarkers and Prev., 5: 355-360 (1996). Examples of biomarkers include genetic markers (e.g., nuclear aberrations [such as micronuclei], gene amplification, and mutation), cellular markers (e.g., differentiation markers and measures of proliferation, such as thymidine labeling index), histologic markers (e.g., premalignant lesions, such as leukoplakia and colonic polyps), and biochemical and pharmacologic markers (e.g., ornithine decarboxylase activity).

Current predictive and prognostic biomarkers include DNA ploidy, S-phase, Ki-67, Her2/neu (c-erb B-2), p53, p21, the retinoblastoma (Rb) gene, MDR-1, bcl-2, cell adhesion molecules, blood group antigens, tumor associated antigens, proliferating antigens, oncogenes, peptide growth factors and their receptors, tumor angiogenesis and angiogenesis inhibitors, and cell cycle regulatory proteins. Beta human chorionic gonadotropin (β-hCG), carcinoembryonic antigen, CA-125, CA 19-9, and others have been evaluated and shown to correlate with clinical response to chemotherapy. See de Vere White, R. W., Stapp, E., “Predicting prognosis in patients with superficial bladder cancer” Oncology (Hunting), 12(12):1717-23; discussion 1724-6 (1998); Stein, J. P. et al., “Prognostic markers in bladder cancer: a contemporary review of the literature” J. Urol.; 160 (3 Pt 1):645-59 (1998); Cook, A. M. et al., “The utility of tumour markers in assessing the response to chemotherapy in advanced bladder cancer” Proc. Annu. Meet. Am. Soc. Clin. Oncol., 17:1199 (1998).

In the case of cancer, molecular markers such as the level of HER2/neu, p53, BCL-2 and estrogen/progesterone receptor expression have been clearly shown to correlate with disease status and progression. This example demonstrates the value of diagnostic and prognostic markers in cancer therapy. Reports from retrospective studies have shown that multivariate predictive models combining existing tumor markers improve cancer detection. See van Haaften-Day C. et al., “OVX1, macrophage-colony stimulating factor, and CA-125-II as tumor markers for epithelial ovarian carcinoma: a critical appraisal”, Cancer (Phila), 92: 2837-44, (2001).

Recent studies have demonstrated that polynucleotide expression information generated by microarray analysis of human tumors can predict clinical outcome (L. J. van't Veer et al., 2002, Nature, 415:530-536; M. West et al., 2001, Proc. Natl. Acad. Sci. USA, 98:11462-11467; T. Sorlie et al., 2001, Proc. Natl. Acad. Sci. USA, 98:10869-10874; M. Shipp et al., 2002, Nature Medicine, 8(1):68-74). These findings bring hope that cancer treatment will be vastly improved by better predicting the response of individual tumors to therapy. Notwithstanding the above references, the scientific literature is innocently silent of any teachings about prognostic biomarkers useful for tailoring a therapeutic protocol involving an iNotch agent for against Notch mediated cellular proliferative disorders.

Although current prognostic criteria and molecular markers provide some guidance in predicting patient outcome and selecting appropriate course of treatment, a significant need exists for a specific and sensitive method for evaluating cancer prognosis and diagnosis, particularly in early-stages. Such a method should specifically distinguish cancer patients with a poor prognosis from those with a good prognosis and permit the identification of high-risk cancer patients who are likely to need aggressive adjuvant therapy.

As well, there is a need for identifying new parameters that can better predict a patient's sensitivity to treatment or therapy. The classification of patient samples is a crucial aspect of cancer diagnosis and treatment. The association of a patient's response to drug treatment with molecular and genetic markers can open up new opportunities for drug development in non-responding patients, or distinguish a drug's indication among other treatment choices because of higher confidence in the efficacy. Further, the pre-selection of patients who are likely to respond well to a medicine, drug, or combination therapy may reduce the number of patients needed in a clinical study or accelerate the time needed to complete a clinical development program (M. Cockett et al., 2000, Current Opinion in Biotechnology, 11:602-609).

Also needed in the art are new and alternative methods and procedures to determine drug sensitivity in patients and which are necessary to treat diseases and disorders, particularly cancers such as those mediated by aberrant Notch signaling, based on patient response at a molecular level. There also remains an unmet need for better ways to detect and diagnose aberrant Notch signaling mediated cellular proliferative disorders, e.g., cancer, including a need for specific biomarkers of the disease.

The present invention aims at overcoming the above deficiencies by providing clinically relevant prognostic and diagnostic tools useful in correlating a patient's response to a chemotherapeutic agent able to modulate Notch signaling as well as identifying patients at risk of failing a therapeutic regiment involving either a particular iNotch agent e.g., a gamma-secretase inhibitor or a test Notch inhibitor. Towards this end, the present invention identifies various genes whose profiles may be used in a clinical setting including predicting a treatment outcome for a patient diagnosed with a Notch mediated cellular proliferative disorder as well as being able to identify potential Notch signally pathway inhibitors based upon expression profiles of some of the early response gene signatures attendant a patient diagnosed with a Notch mediated cancer. Indeed, it is demonstrated in the examples appearing hereunder that the expression profiles of the various genes and/or gene sets detailed herein, individually or collectively with other genes, is predictive of the patient's response to treatment with a Notch inhibitor, such as, for example, a gamma-secretase inhibitor as well as, a instructive of the therapeutic efficacy of a Notch inhibitor in many instances.

### BRIEF SUMMARY OF THE INVENTION

A broad aspect of the invention relates to the identification of biomarker genes (“prognostic markers”) and their use in classifying patients that are likely to respond to treatment with a Notch inhibitor from those that are unlikely to be responsive to treatment with the Notch inhibitor. The assay of the invention can be used prognostically to identify tumors/disease states that have high levels of Notch signaling and could be candidates for therapy with a Notch inhibitor. Alternatively, the assays can also be used to assess the degree of Notch pathway inhibition by anti-Notch drugs, including gamma-secretase inhibitors. The inhibitor need not be limited to a gamma secretase inhibitor. It may include any other Notch signaling inhibitor including an anti-Notch antibody (blocking antibody), an antibody specific for a ligand specific for Notch (neutralizing antibody), a RNAi molecule, an antisense molecule, or any other inhibitor of Notch signaling, including small molecule inhibitors of Notch.

Thus, in one aspect, the gene expression profiles as evidenced by either the nucleic acid expression patterns or polypeptide expression levels attendant one or more of the prognostic biomarker genes disclosed herein correlate with (and thus be able to discriminate) patients with good or poor treatment outcomes. Depending upon the prognostic biomarker. expression levels lower or higher than normal, or a cut-off level are predictive of the patients sensitivity to a Notch inhibitor, such as a gamma secretase. Responsiveness may be viewed in terms of better survival outcomes over time.

The present invention thus provides means for correlating a molecular expression phenotype with a physiological response or lack thereof to a therapeutic moiety. This correlation, in turn, provides a way to molecularly predict the patient's response and/or determine treatment for a cancer afflicted subject. Use of the sequences to identify cells of a sample as responsive, or not, to gamma secretase based treatment may be used to determine the choice, or alteration, of therapy used to treat such cells in the subject, as well as the subject itself, from which the sample originated. As a consequence, the invention provides a non-subjective means of achieving successful preventive intervention in those patients classified as not likely to respond to a specific Notch inhibitor.

The invention in certain aspects thus provides a non-subjective means for the identification of patients with Notch mediated cancer as likely to have a good or poor response outcome to treatment with a notch inhibitor such as gamma secretase by assaying for the expression patterns disclosed herein. As such, where subjective interpretation may have been previously used to determine the prognosis and/or treatment of such cancer patients, the present invention provides objective expression patterns, which may be used alone or in combination with subjective criteria to provide a more accurate assessment of cancer patient outcomes or expected outcomes, including responsiveness to treatment with a particular therapeutic moiety. The expression patterns of the invention thus provide a means to determine cancer prognosis.

The ability to discriminate or identify patients likely to respond (sensitive) to treatment with a Notch inhibitor from those likely to be unresponsive or Notch-inhibitor resistant patient is conferred by the identification of expression of the individual or group of genes or proteins as relevant and not by the form of the assay used to determine the actual level of expression. An assay may utilize any identifying feature of an identified individual gene or protein as disclosed herein or in combination with other genes or encoded proteins as long as the assay reflects, quantitatively or qualitatively, expression of the gene or protein in the “transcriptome” (the transcribed fraction of genes in a genome) or the “proteome” (the translated fraction of expressed genes in a genome). Identifying features include, but are not limited to, unique nucleic acid sequences used to encode (DNA), or express (RNA) said gene or epitopes specific to, or activities of, a protein encoded by said gene. All that is required is the identity of the gene(s) or proteins necessary to identify a potential patient likely to respond to treatment with a Notch-inhibitor or one at-risk of failing a Notch inhibitor, e.g. gamma secretase-based treatment.

The gene expression patterns comprise one or more than one sequence capable of discriminating between cancer treatment outcomes with significant accuracy. The sequences are identified as correlating with cancer treatment outcomes such that the levels of their expression are relevant to a determination of the preferred treatment protocols for a given patient.

In one example, a large sampling of the gene expression profile of a sample is obtained through quantifying the expression levels of mRNA corresponding to many genes. This profile is then analyzed to identify genes or proteins, the expressions of which are positively, or negatively, correlated, with responsiveness to treatment with SAHA. An expression profile of a subset of human proteins or genes may then be identified by the methods of the present invention as correlated with a particular outcome. The use of multiple samples increases the confidence which a gene or sequence may be believed to be correlated with a particular treatment outcome. Without sufficient confidence, it remains unpredictable whether expression of a particular gene or sequence is actually correlated with an outcome and also unpredictable whether expression of a particular gene or protein may be successfully used to identify the outcome for a Notch mediated cancer patient (Notch+ cancer patient). In one embodiment, the Notch mediated cancer is lymphoma. In a particular embodiment, the Notch mediated cancer is cutaneous T cell lymphoma (cancer).

A profile of genes or gene products that are highly correlated with one outcome relative to another may be used to assay a sample from a subject afflicted with cancer to predict the likely responsiveness (or lack thereof) to Notch inhibitor in the subject from whom the sample was obtained. Such an assay may be used as part of a method to determine the therapeutic treatment for said subject based upon the cancer treatment outcome identified.

The correlated genes may be used singly with significant accuracy or in combination to increase the ability to accurately correlate a molecular expression phenotype with a treatment outcome. This correlation is a way to molecularly provide for the determination of survival outcomes and treatment responsiveness as disclosed herein. Additional uses of the correlated gene(s)/proteins are in the classification of cells and tissues; determination of prognosis; and determination and/or alteration of therapy.

In another aspect, the present invention relates to the identification of early response genes or target genes whose gene expression patterns (or profiles or “signatures”) are clinically relevant as risk biomarker for correlating its expression patterns as a potential predictor of therapeutic efficacy of a test Notch inhibitor. Thus, in certain aspects, the invention discloses that low gene expression levels of any one or more of HES5, DTX1, HES4, MYC or SHQ1 post-administration with a Notch inhibitor is correlated with a good prognosis that the Notch inhibitor is therapeutically effective. Similar results have been linked to the use of a gene set comprising at least one or more of HES1, HES4, HES5, HESL, HEY-2, DTX1, MYC, NRARP, PTCRA, SHQ1, whose mean average gene expression level post-administration of the test Notch inhibitors are likely to decrease upon administration of an effective Notch inhibitor. Likewise, higher gene expression levels of at least one of p19, p21 or p27, post-administration of a Notch-inhibitor predicts a better the prognosis that the patient will benefit from treatment with a Notch signaling inhibitor, e.g., the Notch inhibitor is therapeutically effective in inhibiting target genes as evidenced by an increase in the cell cycle genes such as p19, p21 or p27.

In some embodiments, the comparison of the measured value and the reference or control value includes calculating a fold difference between the measured value and the reference value. In some embodiments the measured value is obtained by measuring the level of the prognostic biomarker gene expression in the sample, while in other embodiments the measured value is obtained from a third party.

As used herein, the phrase “fold difference” refers to a numerical representation of the magnitude difference between a measured value and a reference value for either a prognostic biomarker or the early response biomarker gene. Fold difference may be calculated mathematically by division of the numeric measured value with the numeric reference value.

As used herein, a “reference value” or ‘control value” can be an absolute value; a relative value; a value that has an upper and/or lower limit; a range of values; an average value; a median value, a mean value, or a value as compared to a particular control or baseline value. A reference value can be based on an individual sample value, such as for example, a value obtained from a sample from the individual diagnosed with a Notch mediated cancer, but at an earlier point in time such as when determining whether a patient should continue treatment with a Notch inhibitor, or a value obtained from a sample from a patient other than the individual being tested, or a “normal” individual, that is an individual not diagnosed with a Notch mediated cancer. The reference value can be based on a large number of samples, such as from patients diagnosed with a Notch mediated cancer or normal individuals or based on a pool of samples including or excluding the sample to be tested.

In certain aspects, the invention provides for the identification of a gene or protein expression patterns by analyzing gene or protein expression patterns from single cells or homogenous cell populations which have been dissected away from, or otherwise isolated or purified from diseased cancer cells beyond that possible by a simple biopsy. Because the expression of numerous genes and/or proteins fluctuate between cells from different patients as well as between cells from the same patient sample, multiple data from expression of individual genes and/or proteins and gene/protein expression patterns are used as reference data to generate models which in turn permit the identification of individual gene and/or protein(s), the expression of which are most highly correlated with particular treatment outcomes.

In additional embodiments, the invention provides physical and methodological means for detecting the expression of gene(s) identified by the models generated by individual expression patterns. These means may be directed to assaying one or more aspects of the DNA template(s) underlying the expression of the gene(s), of the RNA used as an intermediate to express the gene(s), or of the proteinaceous product expressed by the gene(s).

A broad aspect of the invention, there is provided a method to determine the outcome of a subject afflicted with cancer by assaying a cell containing sample from said subject for expression of one or more of the genes or protein sequences (risk biomarkers) disclosed herein as correlating with responsiveness to a Notch inhibitor based therapy.

The expression levels of the identified sequences may be used alone or in combination with other sequences capable of determining responsiveness to gamma secretase treatment. Preferably, the sequences of the invention are used alone or in combination with each other or other gene sequences, such as in the format of a ratio of expression levels that can have improved predictive power over analysis based on expression of sequences corresponding to individual gene/proteins(s).

The prognostic biomarker gene sequences are one or more of HES1, HES4, HES5, HESL, HEY-2, DTX1, MYC, NRARP, PTCRA and/or SHQ1. Preferred sequences are those identified herein by accession numbers, including splice variants and analogs thereof. Likewise, early response biomarker genes predictive of therapeutic efficacy attendant a Notch inhibitor include at least one gene selected from the group consisting of HES1, HES4, HES5, HESL, HEY-2, DTX1, MYC, NRARP, PTCRA, SHQ1, p19, p21 and/or p27.

As noted supra, an assay of the invention may utilize a means related to the expression level of the sequences disclosed herein as long as the assay reflects, quantitatively or qualitatively, expression of the sequence. Preferably, however, a quantitative assay means is preferred. The ability to determine gamma secretase responsiveness and thus outcome of treatment therewith is provided by the recognition of the relevancy of the level of expression of the identified sequences and not by the form of the assay used to determine the actual level of expression. Stated differently, the invention may be practiced by assaying one or more aspect of the DNA template(s) underlying the expression of the disclosed sequence(s), of the RNA used as an intermediate to express the sequence(s), or of the proteinaceous product expressed by the sequence(s). As such, the detection of the amount of, stability of, or degradation (including rate) of, such DNA, RNA and proteinaceous molecules may be used in the practice of the invention. Thus, for example, the biomarker of the invention may be identified via quantitative analysis of RNA expression using quantitative PCR. It can also be carried out using a Northern blot, microarray analysis, serial analysis of gene expression, nuclease protection assay, or other well known assays. Likewise, protein levels can be assessed by Western blot, immunohistochemistry, ELISA, and/or mass spectroscopy can also be used to assess Notch pathway signaling.

The practice of the present invention is unaffected by the presence of minor mismatches between the disclosed sequences and those expressed by cells of a subject's sample. A non-limiting example of the existence of such mismatches are seen in cases of sequence polymorphisms between individuals of a species, such as individual human patients within Homo sapiens. Knowledge that expression of the disclosed sequences (and sequences that vary due to minor mismatches) is correlated with the presence of non-normal or abnormal cells and cancer is sufficient for the practice of the invention with an appropriate cell containing sample via an assay for expression.

An embodiment of the invention thus provides for the identification of the expression levels of the disclosed sequences by analysis of their expression in a sample of diseased cells. In one preferred embodiment, the sample contains single cells or homogenous cell populations which have been dissected away from, or otherwise isolated or purified from cancer cells beyond that possible by a simple biopsy. Alternatively, un-dissected cells within a “section” of tissue may be used. Multiple means for such analysis are available, including detection of expression within an assay for global, or near global, gene expression in a sample (e.g. as part of a gene expression profiling analysis such as on a microarray) or by specific detection, such as quantitative PCR (Q-PCR), or real time quantitative PCR, Western blot or any other assay well known to one skilled in the art.

The invention also provides a predictor set comprising any one or more of the predictor biomarker genes of the invention. The identified sequences, e.g., polynucleotide or amino acid sequences of any one or more of the risk biomarkers disclosed herein may thus be used in the methods of the invention for predicting a particular patient's responsiveness to SAHA treatment via analysis of lymphoma cells in a tissue or cell containing sample from a subject. As such, the present invention provides a non-empirical means for determining SAHA responsiveness in cancer patients. This provides advantages over the use of a “wait and see” approach following treatment with a Notch inhibitor, e.g., a gamma secretase inhibitor.

In another embodiment, the methods of the invention comprise generating a template profile comprising measurements of levels of at least one or more of the genes or gene sets disclosed herein in a plurality of patients having a chosen prognosis level, e.g., favorable prognosis for treatment with a Notch inhibitor. Thus, such templates are informative of a subset of patients' predicted response to treatment with a particular notch modulating moiety.

It is another aspect of the present invention to provide a method of determining or predicting if an individual requiring drug or chemotherapeutic treatment or therapy for a disease state, or a cancer or tumor of a particular type will successfully respond or will not respond to the drug or chemotherapeutic treatment or therapy prior to the administration of such treatment or chemotherapy. Preferably, the treatment or therapy involves a Notch signaling modulating agent, e.g., an inhibitor of the Notch signaling cascade. Also in accordance with the present invention, cells from a patient tissue sample, e.g., cancer biopsy, are assayed to determine their polynucleotide or polypeptide expression pattern of at least one or more of the prognostic biomarker genes of the invention prior to treatment with a Notch modulating compound or drug, preferably a gamma secretase inhibitor. The resulting polynucleotide expression profile of the test cells before exposure to the compound or drug is compared with the polynucleotide expression pattern of the predictor set of polynucleotides, e.g. control or normal cells.

Success or failure of treatment with a drug can be determined based on the polynucleotide expression pattern of cells from the test tissue (test cells), e.g., a tumor or cancer biopsy, as being relatively similar to or different from the polynucleotide expression pattern of the predictor set of polynucleotides. Thus, if the test cells show a polynucleotide expression profile which corresponds to that of the predictor set of polynucleotides in the control panel of cells which are sensitive to the drug or compound, it is highly likely or predicted that the individual's cancer or tumor will respond favorably to treatment with the drug or compound. By contrast, if the test cells show a polynucleotide expression pattern corresponding to that of the predictor set of polynucleotides of the control panel of cells which are resistant to the drug or compound, it is highly likely or predicted that the individual's cancer or tumor will not respond to treatment with the drug or compound.

In a broad aspect of the invention, there is provided a method for determining or predicting whether an individual requiring therapy for a disease state or disorder such as cancer will or will not respond to treatment, prior to administration of the treatment, wherein the treatment comprises one or more agents that modulate Notch activity. The one or more agents that modulate notch activity can be small molecules or biological molecules. In one aspect, the agent is a small molecule that inhibits NOTCH activity.

Towards this end, the invention provides for the use of the prognostic biomarker genes via determining gene expression or protein expression levels to predict a patient's response or sensitivity to treatment with a Notch inhibitor such as a gamma secretase inhibitor. The data suggest that at least one or more of the prognostic genes are over expressed or exhibit increased expression of said prognostic biomarker genes in such patients and a measurement of their expression levels is predictive of the patient's response to treatment with a Notch inhibitor. In general, increased expression levels of at least one or more genes, individually or cumulatively predict a favorable response meaning that the patient is likely to be sensitive to treatment with the gamma secretase compound.

In yet another aspect, the invention provides a method of monitoring the treatment of a patient having a disease treatable by a compound or agent that modulates a Notch. This can be accomplished by comparing the resistance or sensitivity polynucleotide expression profile of cells obtained from a patient tissue sample, e.g., a tumor or cancer biopsy, prior to treatment with a drug or compound that inhibits the Notch activity. The isolated test cells from the patient's tissue sample are assayed to determine the polynucleotide or polypeptide expression pattern of any one or more of the early response biomarker genes detailed herein. The resulting polynucleotide expression profile of the test cells is compared with the polynucleotide expression pattern in a control sample. Cells expressing higher or lower than normal expression of the polynucleotide or polypeptide expression of the early response gene or protein, predict that the patient is more than likely to respond favorably to treatment with a Notch inhibitor compound. Alternatively, lower than normal expression of at least one or more biomarker protein or polynucleotide indicates that the patient is likely to be resistant to treatment with a Notch signaling inhibitor. Thus, if a patient's response becomes one that is responsive to treatment by a Notch inhibitor compound, based on a correlation of the expression profile of the predictor biomarker, the patient's treatment prognosis can be qualified as favorable and treatment can continue. On the other hand, if the expression profile of the protein biomarker is below that of a control level, this can serve as an indicator that the current treatment should be modified, changed, or even discontinued. Such a monitoring process can indicate success or failure of a patient's treatment with a drug or compound, and the monitoring processes can be repeated as necessary or desired.

An embodiment of the invention is provides for a method for predicting the response of a patient diagnosed with a Notch mediated cancer to treatment with a Notch inhibitor comprising determining the gene expression level of one or more prognostic biomarker genes in a biological sample comprising cancer cells obtained from said subject, wherein the predictive biomarker gene is one or more genes selected from the group consisting of HES1, HES5, and DTX1, wherein gene expression levels of at least one biomarker gene above or below a pre-determined cut-off level is predictive of the patient's treatment response to the anti-cancer agent.

A similar method may be used with a gene set comprising HES1, HES4, HES5, HESL, HEY-2, DTX1, MYC, NRARP, PTCRA and SHQ1 except that the method contemplates obtaining a cumulative gene expression measurement for the gene set followed by determining whether the levels are above or below those of a control or a pre-determined cut-off value. Thus, while individual expression levels of each of HES1, HES5 and DTX1 may be predictive of the patient's sensitivity to the gamma secretase inhibitor compound in certain embodiments, in the above embodiment, it is the cumulative gene expression level of the gene set as a whole that is used to predict the patient's response to treatment with a gamma secretase inhibitor.

Thus, a separate embodiment is directed to a method for predicting the response of a patient diagnosed with a Notch mediated cancer to treatment with a Notch inhibitor, which comprises obtaining a gene expression measurement level of each of a plurality of genes selected from the group consisting of HES1, HES4, HES5, HESL, HEY-2, DTX1, MYC, NRARP, PTCRA and SHQ1 in a biological sample comprising cancer cells obtained from said subject, calculating a mean average expression level from each of said gene expression measurement levels from said plurality of genes, and predicting the response of said patient to treatment with said Notch inhibitor. The step of predicting the response comprises comparing the calculated mean average expression level to a pre-determined cut-off or threshold value/level wherein the patient is predicted to not respond to the treatment protocol when the calculated mean average expression level is below the pre-determined threshold level. Alternatively, the patient is predicted to respond to the treatment protocol when the calculated mean average expression level is equal to or above the pre-determined threshold level. Thus, variation in the gene expression level is predictive of the patients response based upon a measurement for the calculated mean average expression level above or below to a pre-determined level. Gene expression level maybe determined using microarray hybridization, real-time polymerase chain reaction, or northern blot hybridization.

The invention further concerns a prognostic method comprising: (a) subjecting a sample comprising cancer cells obtained from a patient to quantitative analysis of the expression level of the RNA transcript of at least one gene selected from the group consisting of MYC and HES1 or their product, and (b) identifying the patient as likely to have an increased likelihood of responding to a Notch inhibitor if the normalized expression levels of the gene or genes, or their products, are elevated above a defined expression threshold.

Thus, in certain prognostic embodiments, it may be desirable to correct for (normalize away) both differences in the amount of RNA assayed and variability in the quality of the RNA used. On a gene-by-gene basis, measured normalized amount of a patient tumor mRNA is compared to the amount found in a corresponding cancer tissue reference set. The number (N) of cancer tissues in this reference set should be sufficiently high to ensure that different reference sets (as a whole) behave essentially the same way. If this condition is met, the identity of the individual cancer tissues present in a particular set will have no significant impact on the relative amounts of the genes assayed. Unless noted otherwise, normalized expression levels for each mRNA/tested tumor/patient may be expressed as a percentage of the expression level measured in the reference set. Methods of such determination are well known in the art.

In yet another embodiment, the invention provides a method for determining whether a patient presenting with a Notch mediated cellular proliferative disorder is likely to respond to a Notch signaling inhibitor based therapy comprising the steps of:

(a) contacting sample of diseased cell obtained from said patient with a nucleic acid probe that hybridizes to at least one nucleic acid molecule encoding at least one biomarker protein under stringent conditions and detecting a probe-nucleic acid molecule complex;

(b) repeating step (a) wherein the sample is from a normal patient; and

(c) comparing levels of expression of said at least one biomarker protein, wherein an increase in the expression levels of said at least one biomarker protein in said diseased sample indicates that the patient is likely to respond favorably to said Notch inhibitor therapy.

In yet another embodiment the invention provides utilizing the above assays to stratify patient population for a clinical trial.

In certain embodiment, the pre-determined level may comprise a level that is above or below a cut-off. This may include an expression level that is statistically significant, e.g., a p-value of <0.05.

Method of monitoring a patient with a good treatment outcome (good prognosis) from a bad prognosis is also within the scope of the invention. This can be accomplished by comparing the resistance or sensitivity polynucleotide expression profile of cells obtained from a patient tissue sample, e.g., a tumor or cancer biopsy, prior to treatment with a drug or compound that inhibits the Notch activity. The isolated test cells from the patient's tissue sample are assayed to determine the polynucleotide or polypeptide expression pattern of any one or more of the early response biomarker genes detailed herein. The resulting polynucleotide expression profile of the test cells is compared with the polynucleotide expression pattern in a control sample. Cells expressing higher than a reference or control level of the polynucleotide or polypeptide expression of the predictor biomarker genes or proteins, predict that the patient is more than likely to respond favorably to treatment with a Notch inhibitor compound. Thus, if a patient's response becomes one that is responsive to treatment by a Notch inhibitor compound, based on a correlation of the expression profile of the predictor biomarker, the patient's treatment prognosis can be qualified as favorable and treatment can continue. On the other hand, if the expression profile of the protein biomarker is below that of a control level, this can serve as an indicator that the current treatment should be modified, changed, or even discontinued. Such a monitoring process can indicate success or failure of a patient's treatment with a drug or compound, and the monitoring processes can be repeated as necessary or desired.

As noted, supra, it is understood that a gene expression level can be obtained by any method and that the measurement level can be a absolute level, i.e., intensity level, a ratio, i.e., compared to a control level either of a reference gene or the gene itself, or a log ratio. For example, the pre-determined level may comprises performing the same gene expression determination in a control sample of cells and comparing the same to the sample obtained from patient diagnosed with a Notch mediated disorder. The control sample may be a plurality of samples obtained from a single or a plurality of patients that are not diagnosed with Notch mediated cancer (non-diseased cells) or a sample of cells from the same patient comprising cells that do not express aberrant Notch signaling. Alternatively, a control may be derived from patients with a good prognosis. Other controls are within the level of skill level of a skilled clinician.

As used herein, “response” or “responding” includes, for example, a biological response (e.g., a cellular response) or a clinical response (e.g., improved symptoms, a therapeutic effect, or an adverse event) in the mammal.

Another broad aspect of the invention is directed to the identity of responder or early response/target genes, whose individual or cumulative expression levels may be used to assess therapeutic efficacy of a Notch inhibitor. In certain embodiments, one of more of the target genes are over expressed in patients with Notch mediated cancers and their gene expression levels before and after treatment with a Notch-inhibitor may used a to assess the therapeutic efficacy of the particular Notch inhibitors. Consequently, for those gene that are over-expressed relative to a control or pre-determined value will be expected to decrease after treatment with a Notch inhibitor. Therapeutic efficacy is thus hypothesized to occur by inhibition of gene expression with respect to these particular genes. Conversely, if the Notch inhibitor is not therapeutically effective, then the gene expression of these particular early response genes will either remain unchanged or may increase relative to a reference level or the level before administration of the compound.

Also provided is the discovery of certain cell cycle genes exemplified by p19, p21 or p27, whose gene expression levels may also be used to assess the therapeutic efficacy of a Notch inhibitor. Provided herein is data supporting the hypotheses that the gene expression levels, measured, for example, by measuring RNA transcript levels corresponding to one or more of the cell cycle gene or the encoded protein levels, of any one or more of the genes is generally decreased relative to a reference level, e.g., control level with a Notch mediated disorders and that levels of at least one of these target genes should increase or be over-expressed after treatment with a notch inhibitor. Consequently, one way of determining the therapeutic efficacy of treatment protocol with a Notch inhibitor is to assess the expression levels of at least one of the cell cycle genes identified herein before and after treatment with the Notch inhibitor.

Thus, an embodiment of the invention provides a method of predicting the response of a patient diagnosed with a Notch mediated cellular proliferate disorder to a Notch inhibitor, comprising: determining in a biological sample comprising cancer cells obtained from the patient after administration of a therapeutically effective amount of said Notch inhibitor the gene expression level of at least one target gene selected from the group consisting of HES4, HES5, DTX1, MYC, and SHQ1; wherein a change in the gene expression level of said at least one target gene relative to a control correlates with treatment response.

Yet another embodiment of the invention relates to a method of predicting the response of a patient diagnosed with a Notch mediated cellular proliferative disorder to a Notch inhibitor, comprising obtaining a gene expression measurement level for a plurality of genes selected from the group consisting of HES1, HES4, HES5, HESL, HEY-2, DTX1, MYC, NRARP, PTCRA and SHQ1 from a biological sample comprising cancer cells obtained from said subject, prior to and after administration of a Notch inhibitor, calculating an average gene expression level from said plurality of gene expression measurement levels in each of said samples, wherein an decrease in said average gene expression level in the post-dose sample relative to the pre-dose sample is predictive of the patient's treatment response to the Notch inhibitor, means average gene expression level above or below a pre-determined cut-off level correlates with treatment response.

In an alternative embodiment, the post-dose measurement may be compared to a control group comprising non-diseased cells or cells characterized as not exhibiting aberrant Notch signaling.

Another embodiment of the invention provides a method of predicting the response of a patient diagnosed with a Notch mediated cellular proliferative disorder to a Notch inhibitor, comprises determining in a biological sample comprising cancer cells obtained from the a patient after administration of a therapeutically effective amount of said Notch inhibitor the gene expression level of at least one target gene selected from the group consisting of p19, p21 and p27, wherein a change in gene expression level of said at least one target gene above or below a pre-determined cut-off level correlates with treatment response.

In another embodiment, the invention provides a method to determine whether a patient diagnosed with a Notch mediated cancer should continue treatment with a Notch inhibitor, comprising:

(a) determining the level of expression of at least one gene selected from the group consisting of p19, p21 and p27 in a clinical sample of cancer cells obtained from said patient prior to administering a therapeutically effective amount of a Notch inhibitor to said patient to obtain a pre-dosing level and after administration of said Notch inhibitor to obtain a post-dose level, and

(b) comparing the pre-dose and post-dose levels in the sample, wherein an increase in the expression level at least one biomarker gene selected from the group consisting of p19, p21 and p27 is predictive of said patient having a favorable response to treatment with said Notch inhibitor, whereas a decrease in the expression pattern of said at least one biomarker gene is predictive of an unfavorable response to treatment with said Notch inhibitor.

In yet another embodiment, the invention provides a method for determining the therapeutic efficacy of a Notch inhibitor for treating a Notch mediated cellular proliferative disorder comprising assaying a sample of diseased cells from a subject diagnosed with a Notch mediated disorder to determine the gene expression level of each of a plurality of genes selected from the group consisting of HES1, HES4, HES5, HESL, HEY-2, DTX1, MYC, NRARP, PTCRA, SHQ1 and subjecting them to a statistical analysis to obtain a mean average expression level at a first time point after administration of a therapeutically effective amount of a Notch inhibitor, wherein a variation in the mean average level of expression of the plurality of genes at said first time point relative to a control sample is indicative of the therapeutic efficacy of said Notch inhibitor. The above assay may be iterative and the gene expression levels measured at a later point in time may be compared to an earlier time point as a means of comparing the mean average expression level of the entire gene set comprising the above genes. As well, the mean average expression level of the diseased cell sample may be carried out at the same time as calculating the mean average gene expression level of corresponding genes in a control or reference sample.

The gene expression levels of the early response gene may also be used to determine an appropriate dosage level of a Notch inhibitor that will result in effective inhibition of Notch pathway so as to correct aberrant Notch signaling attendant diseased cells. Consequently, in accordance with this embodiment, the invention provides a method of determining a therapeutically effective dosage of a Notch inhibitor to effectively to treat a Notch mediated cellular proliferative disorder in a subject comprising the steps of:

(a) administering to a diseased non-human animal varying dosages of said Notch inhibitor,

(b) determining in a biological sample obtained from said subject after administration of each dosage a gene expression profile of at least one biomarker gene selected from the group consisting of HES1, HES4, HES5, HESL, HEY-2, DTX1, MYC, NRARP, PTCRA, SHQ1, p19, p21, and p27; and

(c) selecting an appropriate dosage based upon the results of the gene expression profile.

Another object of the present invention is to provide one or more specialized microarrays, e.g., oligonucleotide microarrays or cDNA microarrays, comprising those polynucleotides or combinations thereof, as described herein, showing expression profiles that correlate with either sensitivity or resistance to Notch inhibitor compounds. Such microarrays can be employed in in vitro assays for assessing the expression level of the polynucleotides on the microarrays in the test cells from tumor biopsies, for example, and determining whether these test cells will be likely to be resistant or sensitive to the Notch inhibitor compound(s). For example, a specialized microarray can be prepared using some or all of the polynucleotides, polynucleotide subsets, or combinations thereof, as described herein.

In another aspect, the invention provides a kit for predicting treatment outcome or evaluating the treatment outcome of an anti-cancer agent in a patient such as a Notch inhibitor, comprising one or more biomarker genes of the invention. This aspect contemplates a kit comprising a pair of primers for amplification or a probe for hybridization of cDNA of a nucleic acid encoding any one or more of the prognostic RNA transcripts corresponding to the prognostic biomarker genes of the invention, e.g., HES1, HES5 etc. in a biological sample obtained from said patient; and an instructional material for use of the primers or the probe to determine the presence or the absence of the cDNA in the biological sample.

In an alternative embodiment, the kit comprises one or more antibodies having binding specificity to at least one or more of polypeptides encoded by the corresponding biomarker gene in the biological sample from the subject; and an instructional material for use of the antibody(s) to determine the presence or the absence of the polypeptide biomarker in the biological sample

Yet another aspect of the invention proposes developing a cell line expressing any one or more of the prognostic biomarker genes of the invention in order to develop a model to identify potential Notch modulators effective to treat patients expressing higher than normal levels of any one or more of the gene or polypeptide biomarkers of the invention. The cell line may enable one to identify therapeutic moieties capable of eliciting a favorable therapeutic response from otherwise gamma secretase-resistant cells. Animal models following the same protocol are also envisioned by the invention.

It is a further aspect of the present invention to provide a kit for determining or predicting drug susceptibility or resistance by a patient having a disease, with particular regard to a cancer or tumor, namely, a lung cancer or tumor. Such kits are useful in a clinical setting for testing a patient's biopsied tumor or cancer sample, for example, to determine or predict if the patient's tumor or cancer will be resistant or sensitive to a given treatment or therapy with a drug, compound, chemotherapy agent, or biological agent that is directly or indirectly involved with modification, preferably, inhibition, of the activity of a Notch or a cell signaling pathway involving Notch activity. This aspect contemplates a kit comprising a pair of primers for amplification or a probe for hybridization of cDNA of a nucleic acid encoding any one or more polypeptide biomarkers of the invention, e.g., HES1 in a biological sample obtained from said patient; and an instructional material for use of the primers or the probe to determine the presence or the absence of the cDNA in the biological sample. Alternatively, provided in the kit are one or more microarrays, e.g., oligonucleotide microarrays or cDNA microarrays, comprising those polynucleotides that correlate with resistance and sensitivity to Notch modulators, particularly, inhibitors of gamma secretase; and, in suitable containers, the modulator agents/compounds for use in testing cells from patient tissue specimens or patient samples; and instructions for use. In addition, kits contemplated by the present invention can include reagents or materials for the monitoring of the expression of the predictor or marker polynucleotides of the invention at the level of mRNA or encoded protein, using other techniques and systems practiced in the art, e.g., RT-PCR assays, which employ primers designed on the basis of one or more of the predictor polynucleotides described herein, immunoassays, such as enzyme linked immunosorbent assays (ELISAs), immunoblotting, e.g., Western blots, or in situ hybridization, and the like, as further described herein.

The biological sample used in the invention is preferably selected from the group consisting of serum, plasma, and a tissue sample, but generally excludes a normal placental tissue. Those skilled in the art should understand that in the methods of the invention, the “providing a biological sample from a subject” is not a necessary feature to exploit the invention. Therefore, some embodiments of the invention may exclude this step.

While the present invention is described mainly in the context of human cancer, it may be practiced in the context of cancer, lung cancer, colon cancer or any other NOTCH mediated cellular proliferative disorder that is generally responsive to treatment with an NOTCH-inhibitor. Any animal known to be potentially afflicted by cancer may be used.

The cancer can be any types of cancer for example T-ALL. Other types of Notch mediated cancers include cancer cells and tumors expressing aberrant notch signaling. Representative disorders of thus type include breast cancer, ovarian cancer, melanoma, colon cancer, lung cancer, medulloblastoma, glioblastoma neuroblastoma, and pancreatic cancer. See, for example, Miele, Miao et al. (2006).

In a different aspect, the invention concerns a method of preparing a personalized genomics profile for a patient, comprising the steps of: (a) subjecting RNA extracted from a tumor tissue obtained from the patient to gene expression analysis; (b) determining the expression level of one or more the prognostic biomarker genes disclosed herein, wherein the expression level is normalized against a control gene or genes and optionally is compared to the amount found in a Notch mediated cancer reference tissue set; and (c) creating a report summarizing the data obtained by the gene expression analysis.

The report may, for example, include prediction of the likelihood of treatment with a Notch inhibitor (treatment outcome) and/or recommendation for a treatment modality of said patient. Thus, in the foregoing method, if increased expression of one or more of prognostic biomarker genes or the corresponding expression product, is determined, the report includes a prediction that said subject has an increased likelihood of response to chemotherapy comprising a Notch inhibitor.

In this case, in a particular embodiment, the method includes the additional step of treating the patient with a Notch inhibitor.

The biological sample used in the invention is preferably selected from the group consisting of serum, plasma, and a tissue sample. Those skilled in the art should understand that in the methods of the invention, the “providing a biological sample from a subject” is not a necessary feature to exploit the invention. Therefore, some embodiments of the invention may exclude this step.

While the present invention is described mainly in the context of human cancer, it may be practiced in the context of any cellular proliferative disorder that is generally responsive to treatment with a Notch signaling inhibitor. Any animal known to be potentially afflicted by cancer may be used.

### BRIEF DESCRIPTION OF THE DRAWINGS

FIG. 1—Notch signaling pathway activity and response of human T-ALL cell lines to gamma secretase inhibitor using a gene set of 10 genes as detailed in Table 3.

FIG. 2—Gamma secretase inhibitor sensitivity across T-ALL cell lines using transcriptional profiling data, grouped according to gamma secretase inhibitor sensitivity.

FIG. 3—Details the quantification of sensitivity to gamma secretase inhibitor treatment in gamma secretase sensitive cells.

FIG. 4—Notch-10 gene set response in thirteen T-ALL cell lines used for additional gene analysis.

FIG. 5—Heat map of genes which negatively correlated with GSI sensitivity (expression was higher in sensitive cells) and are positively correlated by GSI treatment (expression was diminished in GSI sensitive cells).

FIG. 6—Genes which are positively correlated with GSI-sensitivity of cells (expression was lower in sensitive cells) and negatively correlated with GSI treatment (expression was increased by GSI treatment).

### DETAILED DESCRIPTION OF THE INVENTION

Before the present proteins, nucleotide sequences, and methods are described, it is to be understood that the present invention is not limited to the particular methodologies, protocols, cell lines, vectors, and reagents described, as these may vary. It is also understood that the terminology used herein is for the purpose of describing particular embodiments only, and is not to limit the scope of the present invention.

The singular forms “a,” “an,” and “the” include plural reference unless the context clearly dictates otherwise.

Unless defined otherwise, technical and scientific terms used herein have the same meaning as commonly understood by one of ordinary skill in the art to which this invention belongs. Singleton et al., Dictionary of Microbiology and Molecular Biology 2nd ea., J. Wiley & Sons (New York, N.Y. 1994), and March, Advanced Organic Chemistry Reactions, Mechanisms and Structure 4th ea., John Wiley & Sons (New York, N.Y. 1992), provide one skilled in the art with a general guide to many of the terms used in the present application. The practice of the present invention will employ, unless otherwise indicated, conventional techniques of protein chemistry and biochemistry, molecular biology, microbiology and recombinant DNA technology, which are within the skill of the art. Such techniques are explained fully in the literature.

One skilled in the art will recognize many methods and materials similar or equivalent to those described herein, which could be used in the practice of the present invention. Indeed, the present invention is in no way limited to the methods and materials described. All patents, patent applications, and publications mentioned herein, whether supra or infra, are each incorporated by reference in its entirety.

### A. Definitions

For purposes of the present invention, the following terms are defined below.

The term “polynucleotide,” when used in singular or plural, generally refers to any a polyribonucleotide or polydeoxyribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term ‘polynucleotides” as defined herein. In general, the term “polynucleotide” embraces all chemically, enzymatically and/or metabolically modified forms of unmodified polynucleotides. Polynucleotides can be made by a variety of methods, including in vitro recombinant DNA-mediated techniques and by expression of DNAs in cells and organisms.

The term “microarray” refers to an ordered arrangement of hybridizable array elements, preferably polynucleotide probes, on a substrate.

The terms “differentially expressed gene,” “differential gene expression” and their synonyms, which are used interchangeably, refer to a gene whose expression is activated to a higher or lower level in a subject suffering from a disease, specifically cancer, such as notch meadited cancer, relative to its expression in a normal or control subject. The terms also include genes whose expression is activated to a higher or lower level at different stages of the same disease. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example. Differential gene expression may include a comparison of expression between two or more genes or their gene products, or a comparison of the ratios of the expression between two or more genes or their gene products, or even a comparison of two differently processed products of the same gene, which differ between normal subjects and subjects suffering from a disease, specifically cancer, or between various stages of the saline disease. Differential expression includes both quantitative, as well as qualitative, differences in the temporal or cellular expression pattern in a gene or its expression products among, for example, normal and diseased cells, or among cells which have undergone different disease events or disease stages.

Differential gene expression can, for example, be a measure of the “fold difference” between two samples. Thus, for example, “differential gene expression” may be considered to be present when there is at least an about 1.1, or 1.2 or 1.5-fold difference between the expression of a given gene in normal and diseased subjects, or in various stages of disease development in a diseased subject. Differential gene expression can also be measured using a p-value. When using p-value, a biomarker gene is identified as being differentially expressed as between a first and second population when the p-value is less than 0.1. In certain embodiments the p-value is less than 0.05, while in others it may be lower.

As used herein, the phrase “fold difference” refers to a numerical representation of the magnitude difference between a measured value and a reference value for one or more of the biomarker genes of the invention. Fold difference is calculated mathematically by division of the numeric measured value with the numeric reference value.

“Up-regulated,” as used herein, refers to increased expression of a gene and/or its encoded polypeptide. “Increased expression” refers to increasing (i.e., to a detectable extent) replication, transcription, and/or translation of any of the biomarker genes described herein since up-regulation of any of these processes results in concentration/amount increase of the polypeptide encoded by the gene (nucleic acid). Conversely, “down-regulation,” or “decreased expression” as used herein, refers to decreased expression of a gene and/or its encoded polypeptide. The up-regulation or down-regulation of gene expression can be directly determined by detecting an increase or decrease, respectively, in the level of mRNA for the gene, or the level of protein expression of the gene-encoded polypeptide, using any suitable means known to the art, such as nucleic acid hybridization or antibody detection methods, respectively, and in comparison to controls. In general, the variation in gene expression level is “statistically significant”. Up- or down-regulation may be expressed as a fold-difference, e.g., genes or encoded proteins which demonstrate a e.g., 1.1 fold, 1.2 fold, 1.4 fold, 1.6 fold, 1.8 fold, or more increase or decrease in gene expression (as measured by RNA expression or protein expression), relative to a control.

As used herein, the term “stratifying” refers to sorting individuals into different classes or strata based on the features of a particular disease state or condition. For example, stratifying a population of individuals with Notch mediated cancer involves assigning the individuals on the basis of the severity of the disease (e.g., mild, moderate, advanced, etc.) or tumor classification.

An “individual” is a mammal, more preferably a human. Mammals include, but are not limited to, humans, primates, farm animals, sport animals, rodents and pets. A “normal” individual or sample from a “normal” individual as used herein for quantitative and qualitative data refers to an individual who has or would be assessed by a physician as not having a Notch mediated cellular proliferative disorder or a disorder characterized by aberrant Notch signaling.

According to the invention, a “control level” or “control sample” or “reference level” means a separate baseline level measured in a comparable control cell, which is generally disease free. It may be from the same individual or from another individual who is normal or does not present with the same disease from which the diseased or test sample is obtained. Thus, a “reference value” can be an absolute value; a relative value; a value that has an upper and/or lower limit; a range of values; an average value; a median value, a mean value, or a value as compared to a particular control or baseline value. A reference value can be based on an individual sample value, such as for example, a value obtained from a sample from the individual with a Notch mediated cancer, but at an earlier point in time, or a value obtained from a sample from a patient diagnosed with a Notch cancer other than the individual being tested, or a “normal” individual, that is an individual not diagnosed with a Notch mediated cancer. The reference value can be based on a large number of samples, such as from Notch+patients (patients diagnosed with a Notch mediated cancer) or normal individuals or based on a pool of samples including or excluding the sample to be tested.

The term “normalized” with regard to a gene transcript or a gene expression product refers to the level of the transcript or gene expression product relative to the mean levels of transcripts/products of a set of reference genes, wherein the reference genes are either selected based on their minimal variation across, patients, tissues or treatments (“housekeeping genes”), or the reference genes are the totality of tested genes. In the latter case, which is commonly referred to as “global normalization”, it is important that the total number of tested genes be relatively large, preferably greater than 50. Specifically, the term ‘normalized’ with respect to an RNA transcript refers to the transcript level relative to the mean of transcript levels of a set of reference genes. More specifically, the mean level of an RNA transcript as measured by TaqMan (D RT-PCR refers to the Ct value minus the mean Ct values of a set of reference gene transcripts.

The terms “expression threshold,” and “defined expression threshold” are used interchangeably and refer to the level of a gene or gene product in question above which the gene or gene product serves as a predictive marker for patient response or resistance to a drug. The threshold typically is defined experimentally from clinical studies. The expression threshold can be selected either for maximum sensitivity (for example, to detect all responders to a drug), or for maximum selectivity (for example to detect only responders to a drug), or for minimum error.

The phrase “gene amplification” refers to a process by which multiple copies of a gene or gene fragment are formed in a particular cell or cell line. The duplicated region (a stretch of i amplified DNA) is often referred to as “amplicon.” Often, the amount of the messenger RNA (mRNA) produced, i.e., the level of gene expression, also increases in the proportion to the number of copies made of the particular gene.

The term “prognosis” is used herein to refer to the prediction of the likelihood of cancer attributable death or progression, including recurrence, metastatic spread, and drug resistance, of a neoplastic disease, such as a Notch mediated cancer. As used herein, the term “predicting” or “prediction” refers to making a finding that an individual has a significantly enhanced or reduced probability of an outcome—favorable prognosis versus an unfavorable prognosis. It may also include the likelihood that a Notch inhibitor may be therapeutically effective versus one that is not found to be therapeutic. The term may also be used to refer to the likelihood that a patient will respond either favorably or unfavorably to a drug or set of drugs, and also the extent of those responses, or that a patient will survive, following surgical removal or the primary tumor and/or chemotherapy for a certain period of time without cancer recurrence. The predictive methods of the present invention can be used clinically to make treatment decisions by choosing the most appropriate treatment modalities for any particular patient. Towards this end, the predictive methods of the present invention are valuable tools in predicting if a patient is likely to respond favorably to a treatment regimen, such as chemotherapy with a given drug or drug combination, e.g. gamma secretase inhibitor or another Notch inhibitor, or whether long-term survival of the patient, following a treatment protocol with a Notch inhibitor and/or termination of chemotherapy or other treatment modalities is likely.

The terms “cancer” and “cancerous” refer to or describe the physiological condition in mammals that is typically characterized by unregulated cell growth, e.g., aberrant Notch signaling.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc. “Patient response” can be assessed using any endpoint indicating a benefit to the patient, including, without limitation, (1) inhibition, to some extent, of tumor growth, including slowing down and complete growth arrest; (2) reduction in the number of tumor cells; (3) reduction in tumor size; (4) inhibition (i.e., reduction, slowing down or complete stopping) of tumor cell infiltration into adjacent peripheral organs and/or tissues; (5) inhibition (i.e. reduction, slowing down or complete stopping) of metastasis; (6) enhancement of anti-tumor immune response, which may, but does not have to, result in the regression or rejection of the tumor; (7) relief, to some extent, of one or more symptoms associated with the tumor; (8) increase in the length of survival following treatment; and/or (9) decreased mortality at a given point of time following treatment.

“Neoadjuvant therapy” is adjunctive or adjuvant therapy given prior to the primary (main) therapy. Neoadjuvant therapy includes, for example, chemotherapy, radiation therapy, and hormone therapy. Thus, chemotherapy may be administered prior to surgery to shrink the tumor, so that surgery can be more effective, or in the case of previously unoperable tumors, possible.

“Stringency” of hybridization reactions is readily determinable by one of ordinary skill in the art, and generally is an empirical calculation dependent upon probe length, washing temperature, and salt concentration. Generally, longer probes require higher temperatures for proper annealing, while shorter probes need lower temperatures. Hybridization generally depends on the ability of denatured DNA to re-anneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995); “Stringent conditions” or “high stringency conditions”, as defined herein, typically: (1) employ low ionic strength and high temperature for washing, for example 0.015 M sodium chloride/0.0015 M sodium cikate/0.1% sodium dodecyl sulfate at 50° C.; (2) employ during hybridization a denaturing agent, such as formamide, for example, 50% (v/v) formamide with; 0.1% bovine serum albumin/0.1% Ficoll/0.1% polyvinylpyrrolidone/50 mM sodium phosphate buffer at pH 6.5 with 750 mM sodium chloride, 75 M sodium citrate at 42° C.; or (3) employ; 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 ug/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. “Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 AM NaCl, 15 mM tnsodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

In the context of the present invention, reference to “at least one,” “at least two,” “at least five,” etc. of the genes listed in any particular gene set means any one or any and all combinations of the genes listed.

“Notch” is a membrane-bound transcription factor that regulates many cellular processes, especially in development. In response to ligand binding, its intracellular domain is released by two proteases. The released intracellular domain enters the nucleus and interacts with a DNA-bound protein to activate transcription. The extracellular domain of notch and related proteins contains up to 36 EGF-like domains, followed by three notch (DSL) domains. The intracellular domain contains six ankyrin repeats and a carboxyl-terminal extension that includes a PEST region (rich in proline, glutamine, serine, and threonine). See Kopan R. Notch: a membrane-bound transcription factor. J. Cell Sci. 115: 1095-1097 (2002); Artavanis-tsakonas, et al. Notch signaling: cell fate control and signal integration in development, Science, 284; 770-776 (1999); Mumm, J. S., Kopan, R., “Notch signaling: from the outside in” Dev. Biol., 228: 151-165 (2000), each of which is incorporated by reference herein in its entirety. “Notch” encompasses all members of the Notch receptor family and in particular, Notch1. A description of the Notch signaling pathway and conditions affected by it may be found, for example, in published PCT Applications PCT/GB97/03058, filed on 6 Nov. 1997 and published as WO 98/20142; PCT/GB99/04233, filed on 15 Dec. 1999 and published as WO 00/36089.

Notch inhibiting compounds useful in some or all of the embodiments presented herein are described in WO 01/90084, WO 02/30912, WO 01/70677, WO 03/013506, WO 02/36555, WO 03/093252, WO 03/093264, WO 03/093251, WO 03/093253, WO 2004/039800, WO 2004/039370, WO 2005/030731, WO 2005/014553, U.S. Ser. No. 10/957,251, WO 2004/089911, WO 02/081435, WO 02/081433, WO 03/018543, WO 2004/031137, WO 2004/031139, WO 2004/031138, WO 2004/101538, WO 2004/101539 and WO 02/47671 (including LY-450139) and U.S. Patent Application No. 2003/0114496. See also WO 02/081435 and WO 03/018543. Methods of making and using this inhibitor are described in any one or more of the above recited applications. The contents each of the above referenced applications is incorporated by reference herein in its entirety.

The gamma secretase inhibitor compound useful in some or all of the embodiments referred to herein is described in U.S. Pat. No. 6,984,663; U.S. Ser. No. 11/261,365, the entire contents of each of which is incorporated by reference herein in its entirety.

Quantifying normal levels of the protein biomarker gene or its encoded gene product are well known to a skilled artisan.

Modulated Markers used in the methods of the invention are described in the Examples. The genes that are differentially expressed are either up regulated or down regulated in patients with various lung cancer prognostics. Up regulation and down regulation are relative terms meaning that a detectable difference (beyond the contribution of noise in the system used to measure it) is found in the amount of expression of the genes relative to some baseline. In this case, the baseline is determined based on the algorithm. The genes of interest in the diseased cells are then either up- or down-regulated relative to the baseline level using the same measurement method.

Diseased, in this context, refers to an alteration of the state of a body that interrupts or disturbs, or has the potential to disturb, proper performance of bodily functions as occurs with the uncontrolled proliferation of cells. Someone is diagnosed with a disease when some aspect of that person's genotype or phenotype is consistent with the presence of the disease. However, the act of conducting a diagnosis or prognosis may include the determination of disease/status issues such as determining the likelihood of treatment outcome, type of therapy and therapy monitoring. In therapy monitoring, clinical judgments are made regarding the effect of a given course of therapy by comparing the expression of genes over time to determine whether the gene expression profiles have changed or are changing to patterns more consistent with normal tissue.

Gene expression profiles can also be displayed in a number of ways. The most common method is to arrange raw fluorescence intensities or ratio matrix into a graphical dendogram where columns indicate test samples and rows indicate genes. The data are arranged so genes that have similar expression profiles are proximal to each other. The expression ratio for each gene is visualized as a color. For example, a ratio less than one (indicating down-regulation) may appear in the blue portion of the spectrum while a ratio greater than one (indicating up-regulation) may appear as a color in the red portion of the spectrum. Commercially available computer software programs are available to display such data including “GENESPRING” from Silicon Genetics, Inc. and “DISCOVERY” and “INFER” software from Partek, Inc.

In the case of measuring protein levels to determine gene expression, any method known in the art is suitable provided it results in adequate specificity and sensitivity. For example, protein levels can be measured by binding to an antibody or antibody fragment specific for the protein and measuring the amount of antibody-bound protein. Antibodies can be labeled by radioactive, fluorescent or other detectable reagents to facilitate detection. Methods of detection include, without limitation, enzyme-linked immunosorbent assay (ELISA) and immunoblot techniques.

### B. Detailed Description

A Biomarker is any indicia of the level of expression of an indicated marker gene. The indicia can be direct or indirect and measure over- or under-expression of the gene given the physiologic parameters and in comparison to an internal control, normal tissue or another carcinoma. Biomarkers include, without limitation, nucleic acids (both over and under-expression and direct and indirect). Using nucleic acids as Biomarkers can include any method known in the art including, without limitation, measuring DNA amplification, RNA, micro RNA, loss of heterozygosity (LOH), single nucleotide polymorphisms (SNPs, Brookes (1999)), microsatellite DNA, DNA hypo- or hyper-methylation. Using proteins as Biomarkers can include any method known in the art including, without limitation, measuring amount, activity, modifications such as glycosylation, phosphorylation, ADP-ribosylation, ubiquitination, etc., immunohistochemistry (IHC).

The biomarker genes provided herein are those associated with a particular tumor or tissue type. These biomarker gene may be associated with numerous cancer types but provided that the expression of the gene is sufficiently associated with one tumor or tissue type to be identified using methods known to one skilled in art, the gene can be using in the claimed invention to determine cancer status, prognosis (treatment outcome) and therapeutic efficacy of a test Notch inhibitor. In the main, the invention provides the identity of preferred biomarker genes including combinations thereof—gene sets as detailed in Table 3, the expression patterns of which have clinical significance relating to Notch mediated cancers. The preferred gene(s) according to the invention corresponds to the sequence designated by Accession Number or a SEQ ID NO when it contains that sequence. A gene segment or fragment corresponds to the sequence of such gene when it contains a portion of the referenced sequence or its complement sufficient to distinguish it as being the sequence of the gene. A gene expression product corresponds to such sequence when its RNA, mRNA, or cDNA hybridizes to the composition having such sequence (e.g. a probe) or, in the case of a peptide or protein, it is encoded by such mRNA. A segment or fragment of a gene expression product corresponds to the sequence of such gene or gene expression product when it contains a portion of the referenced gene expression product or its complement sufficient to distinguish it as being the sequence of the gene or gene expression product.

The inventive methods, compositions, articles, and kits of described and claimed in this specification include one or more Marker genes. “Marker” or “Marker gene” “biomarker gene” is used throughout this specification refers to genes and/or gene sets and gene expression products that correspond with any gene the over- or under-expression of which is associated with a tumor or tissue type. The preferred Marker genes are described in more detail herein. See, for example, Table 3.

Genes can be grouped so that information obtained about the set of genes in the group provides a sound basis for making a clinically relevant judgment such as a diagnosis, prognosis, or treatment choice. Certain embodiments of the invention comprise sets of genes that make up a particular gene set or combination. As with most biomarkers, it may be desirable to use the fewest number of markers sufficient to make a correct medical judgment. This prevents a delay in treatment pending further analysis as well unproductive use of time and resources.

One method of establishing gene expression portfolios is through the use of optimization algorithms such as the mean variance algorithm widely used in establishing stock portfolios. This method is described in detail in US Patent Publication Number 20030194734. Essentially, the method calls for the establishment of a set of inputs (stocks in financial applications, expression as measured by intensity here) that will optimize the return (e.g., signal that is generated) one receives for using it while minimizing the variability of the return. Many commercial software programs are available to conduct such operations. “Wagner Associates Mean-Variance Optimization Application,” referred to as “Wagner Software” throughout this specification, is preferred. This software uses functions from the “Wagner Associates Mean-Variance Optimization Library” to determine an efficient frontier and optimal portfolios in the Markowitz sense is one option. Use of this type of software requires that microarray data be transformed so that it can be treated as an input in the way stock return and risk measurements are used when the software is used for its intended financial analysis purposes. Various other methods are within the level of skill of one skilled in the art of molecular medicine.

The process of selecting a portfolio can also include the application of heuristic rules. Such rules are formulated based on biology and an understanding of the technology used to produce clinical results. In certain embodiments, they are applied to output from the optimization method. For example, the mean variance method of portfolio selection can be applied to microarray data for a number of genes differentially expressed in subjects with cancer. Output from the method would be an optimized set of genes that could include some genes that are expressed in say peripheral blood as well as in diseased tissue. If samples used in the testing method are obtained from peripheral blood and certain genes differentially expressed in instances of cancer could also be differentially expressed in peripheral blood, then a heuristic rule can be applied in which a portfolio is selected from the efficient frontier excluding those that are differentially expressed in peripheral blood. Of course, the rule can be applied prior to the formation of the efficient frontier by, for example, applying the rule during data pre-selection.

Other heuristic rules can be applied that are not necessarily related to the biology in question. For example, one can apply a rule that only a prescribed percentage of the portfolio can be represented by a particular gene or group of genes. Commercially available software such as the Wagner Software readily accommodates these types of heuristics. This can be useful, for example, when factors other than accuracy and precision (e.g., anticipated licensing fees) have an impact on the desirability of including one or more genes.

The gene expression profiles of the invention can also be used in conjunction with other non-genetic diagnostic methods useful in cancer diagnosis, prognosis, or treatment monitoring. For example, in some circumstances it is beneficial to combine the prognostic power of the gene expression based methods described above with data from conventional markers such as serum protein markers (e.g., Cancer Antigen 27.29 (“CA 27.29”)). A range of such markers exist including such analytes as CA 27.29. In one such method, blood is periodically taken from a treated patient and then subjected to an enzyme immunoassay for one of the serum markers described above. When the concentration of the marker suggests the return of tumors or failure of therapy, a sample source amenable to gene expression analysis is taken. Where a suspicious mass exists, a fine needle aspirate (FNA) is taken and gene expression profiles of cells taken from the mass are then analyzed as described above. Alternatively, tissue samples may be taken from areas adjacent to the tissue from which a tumor was previously removed. This approach can be particularly useful when other testing produces ambiguous results.

Among the various objects disclosed herein, a broad aspect relates to a prognostic method of predicting a patient's response to treatment with a Notch inhibitor by obtaining a biological sample from a cancer patient; and measuring Biomarkers associated with Marker genes corresponding to those selected from Table 3 where the expression levels of the Marker genes above or below pre-determined cut-off levels are indicative of cancer status.

In various distinct embodiments, the present invention is based, in part, on the identification of reliable prognostic for the improved prediction of treatment outcome of a patient diagnosed with a Notch mediated cellular proliferative disorder with a Notch inhibitor. The invention provides a population of reliable genomic target genes and their attendant sequences for use in prognostic methods provided by the present invention, which have been designated herein as HES1, HES4, HES5, HESL, HEY-2, DTX1, MYC, NRARP, PTCRA and SHQ1, p19, p21 and p27, including combinations thereof. The method proposes measuring the amount of one or more prognostic Marker genes in a sample of diseased cells obtained from a patient diagnosed with a Notch mediated cancer and comparing the measured amount with a reference value for each one or more of the Markers of the invention. In some instances, the reference value is a pre-determined value wherein a measured value above or below the pre-determined cut-off value is prognostic of the patient's treatment response or outcome to treatment with the gamma secretase inhibitor. In other aspects, the measured value is compared to a reference or control value. The information thus obtained may be used to aid in the patient's prognosis relative to the treatment protocol.

Accordingly, the present invention provides a method for predicting the response of a patient diagnosed with a Notch mediated cancer to treatment with a Notch inhibitor comprising determining the gene expression level of one or more prognostic biomarker genes in a biological sample comprising cancer cells obtained from said subject, wherein the predictive biomarker gene is one or more gene selected from the group consisting of HES1, HES5, and DTX1, wherein gene expression levels above or below a pre-determined cut-off level is predictive of the patient's treatment response to the anti-cancer agent.

In some embodiments, the above method includes comparing the measured level of at least one prognostic biomarker in a biological sample from an individual to a reference level for the biomarker and making a prediction relative to treatment outcome based upon the results obtained, wherein an increase in the level of at least one of the prognostic biomarkers indicates that the patient is likely to respond to treatment with the Notch inhibitor.

In some examples, the invention includes obtaining a gene expression measurement level of each of a plurality of genes selected from the group consisting of HES1, HES4, HES5, HESL, HEY-2, DTX1, MYC, NRARP, PTCRA and SHQ1 in a biological sample comprising cancer cells obtained from said subject, calculating a mean average expression level from each of said gene expression measurement levels from said plurality of genes, and predicting the response of said patient to treatment with said Notch inhibitor wherein said predicting comprises comparing said calculated mean average expression level to a pre-determined threshold value wherein said patient is predicted to not respond to said treatment when said calculated mean average expression level is below said pre-determined threshold level or said patient is predicted to respond to said treatment when said calculated mean average expression level is equal to or above said pre-determined threshold level.

In another embodiment, the above method includes a plurality of genes comprising one of MYC and HES1, wherein an increase in the calculated mean average expression level of a MYC and HES1 combined when compared to a reference level is increased, thus providing the basis for the prediction that the patients is likely to have a good prognosis upon treatment with a Notch inhibitor, e.g., gamma secretase inhibitor.

In another embodiment, the invention provides the identity of genes or gene sets, which expression pattern can correlate with the therapeutic efficacy of a test Notch inhibitor. These genes for the purposes of these embodiments are referred to as early response or target genes. Essentially, the data show that gene expression levels of such genes, either alone or cumulatively are generally up regulated in patients diagnosed with a Notch cancer and upon administration of a therapeutically effective Notch inhibitor, these should normally be down-regulated relative to a control or a pre-dose sample. As such, if the test Notch inhibitor were to be therapeutically effective, it would downregulates expression of at least one or more of these genes in a patient diagnosed with a notch cancer. Thus, when the calculated average mean expression level of a group of genes represented by HES1, HES4, HES5, HESL, HEY-2, DTX1, MYC, NRARP, PTCRA and SHQ1 is measured and compared to that of a control, then the levels after administration of the test Notch inhibitor results in the inhibition of these genes such that there is a decrease in the calculated mean average expression level relative to a control or a pre-dose sample. As such, gene expression profiles of at least one or more of these early response Markers can be used as a target to assess whether a test Notch inhibitor is actually inhibiting Notch signaling or otherwise being therapeutically effective. In a related embodiment, the method uses the expression pattern of at lest one of HES4, HES5, DTX1, MYC, and SHQ1, wherein a decrease post-dose of a test Notch inhibitor indicates that the inhibitor is effective in inhibiting Notch signaling.

On the other hand, the gene expression levels of at least one cell cycle gene can also be used to assess the therapeutic efficacy of a Notch inhibitor except in the case of the cell cycle genes, such as p19, p21 and p27, an increase in the gene expression level of at least one of these Markers is indicative the therapeutic efficacy of the test Notch inhibitor. In additional examples, comparing the measured level to a reference level for each one or more of the prognostic biomarker genes or early response genes measured comprises calculating the fold difference between the measured level and the reference level. In some examples, a method further comprises comparing the fold difference for each one or more of the biomarker genes on the invention measured with a minimum fold difference level. In some examples, the method further comprises the step of obtaining a value for the comparison of the measured level to the reference level. Also provided herein are computer readable formats comprising the values obtained by the method as described herein.

In certain aspects of the invention, measured values for at least one gene from Table 3 from one or more individuals are compared, wherein biomarkers that vary significantly are useful for aiding in the prognosis, stratification, monitoring, and/or prediction of treatment outcome. In further aspects of the invention, levels of a set of genes, Table 3, gene set comprising 10 genes, from one or more individuals are measured to produce measured values, wherein biomarkers that vary significantly are useful for aiding in the stratification, monitoring, and/or prediction of treatment outcome.

The process of comparing the measured values may be carried out by any method known in the art, including Significance Analysis of Microarrays, Tree Harvesting, CART, MARS, Self Organizing Maps, Frequent Item Set, or Bayesian networks.

In a further aspect, the invention provides methods for identifying at least one biomarker useful for the stratification of a patient population for a clinical trial by obtaining measured values from each of a for a plurality of biomarkers, wherein the set of peripheral biological fluid samples is divisible into subsets on the basis of strata of a neurological disease, comparing the measured values from each subset for at least one biomarker; and identifying biomarkers for which the measured values are significantly different between the subsets.

In another aspect, the invention provides methods for identifying at least one biomarker useful for the monitoring of a neurological disease by obtaining measured values from a set of peripheral biological fluid samples for a plurality of biomarkers, wherein the patient population is stratified based upon the results of the gene expression profile, comparing the measured values from each subset for at least one biomarker; and identifying biomarkers for which the measured values are significantly different between the patient sample and a control or a pre-determined cut-off value.

Alternatively, the Notch mediated cancer can also be staged based upon the expression levels of the marker genes detailed herein. The stage can correspond to any classification system, including to patients with similar gene expression profiles.

In the methods of the invention, the pre-determined cut-off levels have at least a statistically significant p-value over-expression in the sample having metastatic cells relative to benign cells or normal tissue, preferably the p-value is less than 0.05.

In the methods of the invention, gene expression can be measured by any method known in the art, including, without limitation on a microarray or gene chip, nucleic acid amplification conducted by polymerase chain reaction (PCR) such as reverse transcription polymerase chain reaction (RT-PCR), measuring or detecting a protein encoded by the gene such as by an antibody specific to the protein or by measuring a characteristic of the gene such as DNA amplification, methylation, mutation and allelic variation. The microarray can be for instance, a cDNA array or an oligonucleotide array. All these methods and can further contain one or more internal control reagents.

Preferred methods for establishing gene expression profiles include determining the amount of RNA that is produced by a gene that can code for a protein or peptide. This is accomplished by reverse transcriptase PCR (RT-PCR), competitive RT-PCR, real time RT-PCR, differential display RT-PCR, Northern Blot analysis and other related tests. While it is possible to conduct these techniques using individual PCR reactions, it is best to amplify complementary DNA (cDNA) or complementary RNA (cRNA) produced from mRNA and analyze it via microarray. A number of different array configurations and methods for their production are known to those of skill in the art and are described in U.S. patents such as: U.S. Pat. Nos. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; and 5,700,637.

Compositions comprising at least one probe set selected from the group consisting of: Marker genes selected from the group consisting of those disclosed in Table 3.

The present invention provides articles for assessing Notch cancer status comprising: materials for detecting isolated nucleic acid sequences, their complements, or portions thereof of a combination of genes selected from the group consisting of Marker genes corresponding to those selected from Table 3. The articles can further contain reagents for conducting a microarray analysis and/or a medium through which said nucleic acid sequences, their complements, or portions thereof are assayed.

Articles of this invention include representations of the gene expression profiles useful for prognosticating, monitoring, and otherwise assessing diseases. These profile representations are reduced to a medium that can be automatically read by a machine such as computer readable media (magnetic, optical, and the like). The articles can also include instructions for assessing the gene expression profiles in such media. For example, the articles may comprise a CD ROM having computer instructions for comparing gene expression profiles of the portfolios of genes described above. The articles may also have gene expression profiles digitally recorded therein so that they may be compared with gene expression data from patient samples. Alternatively, the profiles can be recorded in different representational format. A graphical recordation is one such format. Clustering algorithms such as those incorporated in “DISCOVERY” and “INFER” software from Partek, Inc. mentioned above can best assist in the visualization of such data.

Alternatively, articles according to the invention can be fashioned into reagent kits for conducting hybridization, amplification, and signal generation indicative of the level of expression of the genes of interest for detecting cancer

The present invention provides a kit for conducting an assay to determine Notch cancer prognosis in a biological sample comprising: materials for detecting isolated nucleic acid sequences, their complements, or portions thereof of a combination of genes selected from the group consisting of Marker genes corresponding to those selected from Table 3. The kit can further comprise reagents for conducting a microarray analysis, and/or a medium through which said nucleic acid sequences, their complements, or portions thereof are assayed.

Kits made according to the invention include formatted assays for determining the gene expression profiles. These can include all or some of the materials needed to conduct the assays such as reagents and instructions and a medium through which Biomarkers are assayed.

The present invention also provides a microarray or gene chip for performing the methods of the invention. The microarray can contain isolated nucleic acid sequences, their complements, or portions thereof of a combination of genes selected from the group consisting of Marker genes corresponding to those selected from Table 3. Preferably, the microarray is capable of measurement or characterization of at least 1.5-fold over- or under-expression. Preferably, the microarray provides a statistically significant p-value over- or under-expression. Preferably, the p-value is less than 0.05. The microarray can contain a cDNA array or an oligonucleotide array and/or one or more internal control reagents.

The mere presence or absence of particular nucleic acid sequences in a tissue sample has only rarely been found to have diagnostic or prognostic value. Information about the expression of various proteins, peptides or mRNA, on the other hand, is increasingly viewed as important. The mere presence of nucleic acid sequences having the potential to express proteins, peptides, or mRNA (such sequences referred to as “genes”) within the genome by itself is not determinative of whether a protein, peptide, or mRNA is expressed in a given cell. Whether or not a given gene capable of expressing proteins, peptides, or mRNA does so and to what extent such expression occurs, if at all, is determined by a variety of complex factors. Irrespective of difficulties in understanding and assessing these factors, assaying gene expression can provide useful information about the occurrence of important events such as tumorogenesis, metastasis, apoptosis, and other clinically relevant phenomena. Relative indications of the degree to which genes are active or inactive can be found in gene expression profiles. The gene expression profiles of this invention are used to provide diagnosis, status, prognosis and treatment protocol for lung cancer patients.

Sample preparation requires the collection of patient samples. Patient samples used in the inventive method are those that are suspected of containing diseased cells from patients diagnosed with or suspected of presenting with a cancer characterized by aberrant Notch-signaling. Bulk tissue preparation obtained from a biopsy or a surgical specimen and Laser Capture Microdissection (LCM) are also suitable for use. LCM technology is one way to select the cells to be studied, minimizing variability caused by cell type heterogeneity. Consequently, moderate or small changes in Marker gene expression between normal or benign and cancerous cells can be readily detected. Once the sample containing the cells of interest has been obtained, a gene expression profile is obtained using a Biomarker, for genes in the appropriate portfolios.

Microarray technology allows for the measurement of the steady-state mRNA level of thousands of genes simultaneously thereby presenting a powerful tool for identifying effects such as the onset, arrest, or modulation of uncontrolled cell proliferation. Two microarray technologies are currently in wide use. The first are cDNA arrays and the second are oligonucleotide arrays. Although differences exist in the construction of these chips, essentially all downstream data analysis and output are the same. The product of these analyses are typically measurements of the intensity of the signal received from a labeled probe used to detect a cDNA sequence from the sample that hybridizes to a nucleic acid sequence at a known location on the microarray. Typically, the intensity of the signal is proportional to the quantity of cDNA, and thus mRNA, expressed in the sample cells. A large number of such techniques are available and useful. Methods for determining gene expression can be found in U.S. Pat. Nos. 6,271,002; 6,218,122; 6,218,114; and 6,004,755.

Analysis of the expression levels in some instances may be conducted by comparing such signal intensities. This is best done by generating a ratio matrix of the expression intensities of genes in a test sample versus those in a control sample. For instance, the gene expression intensities from a diseased tissue can be compared with the expression intensities generated from benign or normal tissue of the same type. A ratio of these expression intensities indicates the fold-change in gene expression between the test and control samples.

The present invention also provides a diagnostic/prognostic portfolio comprising isolated nucleic acid sequences, their complements, or portions thereof of a combination of genes selected from the group consisting of Marker genes corresponding to those selected from Table 3. Preferably, the portfolio is capable of measurement or characterization of at least 1.5-fold over- or under-expression. Preferably, the portfolio provides a statistically significant p-value over- or under-expression. Preferably, the p-value is less than 0.05.

Representative methods of gene profiling techniques are provided herein, it being understood that variations to these methods are also encompassed by the invention as are other methods of qualifying and/or quantifying gene expression levels known to a skilled artisan.

1. Gene Expression Profiling

In general, methods of gene expression profiling can be divided into two large groups: methods based on hybridization analysis of polynucleotides, and methods based on sequencing of polynucleotides. The most commonly used methods known in the art for the quantification of mRNA expression in a sample include northern blotting and in situ hybridization (Parker & Barnes, Methods in Molecular Biology 106:247-283 (1999)); RNAse protection assays (Hod, Biotechniques 13:852-854 (1992)); and reverse transcription polymerase chain reaction (RT-PCR) (Weis et al., Trends in Genetics 8:263-264 (1992)). Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS).

2. Reverse Transcriptase PCR (RT-PCR)

Of the techniques listed above, the most sensitive and most flexible quantitative method is RT-PCR, which can be used to compare mRNA levels in different sample populations, in normal and tumor tissues, with or without drug treatment, to characterize patterns of gene expression, to discriminate between closely related mRNAs, and to analyze RNA structure.

The first step is the isolation of mRNA from a target sample. The starting material is typically total RNA isolated from human tumors or tumor cell lines, and corresponding normal tissues or cell lines, respectively. Thus RNA can be isolated from a variety of primary tumors, including breast, lung, colon, prostate, brain, liver, kidney, pancreas, spleen, thymus, testis, ovary, uterus, etc., tumor, or tumor cell lines, with pooled DNA from healthy donors. If the source of mRNA is a primary tumor, mRNA can be extracted, for example, from frozen or archived paraffin-embedded and fixed (e.g. formalin-fixed) tissue samples.

General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andrs et al., BioTechniques 18:42044 (1995). In particular, RNA isolation can be performed using purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MasterPure™ Complete DNA and RNA Purification Kit (EPICENTRE®, Madison, Wis.), and Paraffin Block RNA Isolation Kit (Ambion, Inc.). Total RNA from tissue samples can be isolated using RNA Stat-60 (Tel-Test). RNA prepared from tumor can be isolated, for example, by cesium chloride density gradient centrifugation.

As RNA cannot serve as a template for PCR, the first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan® RT-PCR can be performed using commercially available equipment, such as, for example, ABI PRISM 7700™ Sequence Detection System™ (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700™ Sequence Detection System. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

5′-Nuclease assay data are initially expressed as Cₜ, or the threshold cycle. As discussed above, fluorescence values are recorded during every cycle and represent the amount of product amplified to that point in the amplification reaction. The point when the fluorescent signal is first recorded as statistically significant is the threshold cycle (Cₜ).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and β-actin.

A more recent variation of the RT-PCR technique is the real time quantitative PCR, which measures PCR product accumulation through a dual-labeled fluorigenic probe (i.e., TaqMan® probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996).

The steps of a representative protocol for profiling gene expression using fixed, paraffin-embedded tissues as the RNA source, including mRNA isolation, purification, primer extension and amplification are given in various published journal articles. See for example: T. E. Godfrey et al, J. Molec. Diagnostics 2: 84-91 [2000]; K. Specht et al., Am. J. Pathol. 158: 419-29 [2001]. Briefly, a representative process starts with cutting about 10 μ.m thick sections of paraffin-embedded tumor tissue samples. The RNA is then extracted, and protein and DNA are removed. After analysis of the RNA concentration, RNA repair and/or amplification steps may be included, if necessary, and RNA is reverse transcribed using gene specific promoters followed by RT-PCR.

According to one aspect of the present invention, PCR primers and probes are designed based upon intron sequences present in the gene to be amplified. In this embodiment, the first step in the primer/probe design is the delineation of intron sequences within the genes. This can be done by publicly available software, such as the DNA BLAT software developed by Kent, W. J., Genome Res. 12(4):656-64 (2002), or by the BLAST software including its variations. Subsequent steps follow well established methods of PCR primer and probe design.

In order to avoid non-specific signals, it is important to mask repetitive sequences within the introns when designing the primers and probes. This can be easily accomplished by using the Repeat Masker program available on-line through the Baylor College of Medicine, which screens DNA sequences against a library of repetitive elements and returns a query sequence in which the repetitive elements are masked. The masked intron sequences can then be used to design primer and probe sequences using any commercially or otherwise publicly available primer/probe design packages, such as Primer Express (Applied Biosystems); MGB assay-by-design (Applied Biosystems); Primer3 (Steve Rozen and Helen J. Skaletsky (2000) Primer3 on the WWW for general users and for biologist programmers. In: Krawetz S, Misener S (eds) Bioinformatics Methods and Protocols: Methods in Molecular Biology. Humana Press, Totowa, N.J., pp 365-386).

The most important factors considered in PCR primer design include primer length, melting temperature (Tm), and G/C content, specificity, complementary primer sequences, and 3′-end sequence. In general, optimal PCR primers are generally 17-30 bases in length, and contain about 20-80%, such as, for example, about 50-60% G+C bases. Tm's between 50 and 80.degree C., e.g. about 50 to 70.degree C., are typically preferred.

For further guidelines for PCR primer and probe design see, e.g. Dieffenbach, C. W. et al., “General Concepts for PCR Primer Design” in: PCR Primer, A Laboratory Manual, Cold Spring Harbor Laboratory Press, New York, 1995, pp. 133-155; Innis and Gelfand, “Optimization of PCRs” in: PCR Protocols, A Guide to Methods and Applications, CRC Press, London, 1994, pp. 5-11; and Plasterer, T. N. Primerselect: Primer and probe design. Methods Mol. Biol. 70:520-527 (1997), the entire disclosures of which are hereby expressly incorporated by reference.

3. Microarrays

Differential gene expression can also be identified, or confirmed using the microarray technique. Thus, the expression profile of breast cancer-associated genes can be measured in either fresh or paraffin-embedded tumor tissue, using microarray technology. In this method, polynucleotide sequences of interest (including cDNAs and oligonucleotides) are plated, or arrayed, on a microchip substrate. The arrayed sequences are then hybridized with specific DNA probes from cells or tissues of interest. Just as in the RT-PCR method, the source of mRNA typically is total RNA isolated from human tumors or tumor cell lines, and corresponding normal tissues or cell lines. Thus RNA can be isolated from a variety of primary tumors or tumor cell lines. If the source of mRNA is a primary tumor, mRNA can be extracted, for example, from frozen or archived paraffin-embedded and fixed (e.g. formalin-fixed) tissue samples, which are routinely prepared and preserved in everyday clinical practice.

In an embodiment of the microarray technique, PCR amplified inserts of cDNA clones are applied to a substrate in a dense array. In some embodiments, at least 10,000 nucleotide sequences may be applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93(2):106-149 (1996)). Microarray analysis can be performed by commercially available equipment, following manufacturer's protocols, such as by using the Affymetrix GenChip technology, or Incyte's microarray technology.

The development of microarray methods for large-scale analysis of gene expression makes it possible to search systematically for molecular markers of cancer classification and outcome prediction in a variety of tumor types.

4. Serial Analysis of Gene Expression (SAGE)

Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, e.g. Velculescu et al., Science 270:484-487 (1995); and Velculescu et al., Cell 88:243-51 (1997).

5. MassARRAY Technology

The MassARRAY (Sequenom, San Diego, Calif.) technology is an automated, high-throughput method of gene expression analysis using mass spectrometry (MS) for detection. According to this method, following the isolation of RNA, reverse transcription and PCR amplification, the cDNAs are subjected to primer extension. The cDNA-derived primer extension products are purified, and dispensed on a chip array that is pre-loaded with the components needed for MALTI-TOF MS sample preparation. The various cDNAs present in the reaction are quantitated by analyzing the peak areas in the mass spectrum obtained.

6. Gene Expression Analysis by Massively Parallel Signature Sequencing (MPSS)

This method, described by Brenner et al., Nature Biotechnology 18:630-634 (2000), is a sequencing approach that combines non-gel-based signature sequencing with in vitro cloning of millions of templates on separate 5 μm diameter microbeads. First, a microbead library of DNA templates is constructed by in vitro cloning. This is followed by the assembly of a planar array of the template-containing microbeads in a flow cell at a high density (typically greater than 3.times.10.sup.6 microbeads/cm.sup.2). The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a yeast cDNA library.

7. Immunohistochemistry

Immunohistochemistry methods are also suitable for detecting the expression levels of the prognostic markers of the present invention. Thus, antibodies or antisera, preferably polyclonal antisera, and most preferably monoclonal antibodies specific for each marker are used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

8. Proteomics

The term “proteome” is defined as the totality of the proteins present in a sample (e.g. tissue, organism, or cell culture) at a certain point of time. Proteomics includes, among other things, study of the global changes of protein expression in a sample (also referred to as “expression proteomics”). Proteomics typically includes the following steps: (1) separation of individual proteins in a sample by 2-D gel electrophoresis (2-D PAGE); (2) identification of the individual proteins recovered from the gel, e.g. my mass spectrometry or N-terminal sequencing, and (3) analysis of the data using bioinformatics. Proteomics methods are valuable supplements to other methods of gene expression profiling, and can be used, alone or in combination with other methods, to detect the products of the prognostic markers of the present invention.

9. General Description of the mRNA Isolation, Purification and Amplification

The steps of a representative protocol for profiling gene expression using fixed, paraffin-embedded tissues as the RNA source, including mRNA isolation, purification, primer extension and amplification are given in various published journal articles {for example: T. E. Godfrey et al. J. Molec. Diagnostics 2: 84-91 [2000]; K. Specht et al., Am. J. Pathol. 158: 419-29 [2001]}. Briefly, a representative process starts with cutting about 10 μm thick sections of paraffin-embedded tumor tissue samples. The RNA is then extracted, and protein and DNA are removed. After analysis of the RNA concentration, RNA repair and/or amplification steps may be included, if necessary, and RNA is reverse transcribed using gene specific promoters followed by RT-PCR. Finally, the data are analyzed to identify the best treatment option(s) available to the patient on the basis of the characteristic gene expression pattern identified in the tumor sample examined.

The invention is further illustrated by the following non-limiting examples. All references cited herein are hereby incorporated herein.

The invention is further illustrated by the following non-limiting examples. All references cited herein are hereby incorporated herein.

### EXAMPLES

### Example 1

### Materials and Methods

Compounds: MRK-003 (active GSI) and MRK-006 (275-fold less active enantiomer control) were previously described (Lewis, Leveridge et al. 2007).

Cell culture and cell viability: Human T-ALL cell lines were purchased from ATCC (Manassas, Va.) or DSMZ (Braunschweig, Germany). Cell lines were maintained in RPMI supplemented with 10-15% FBS and 2 mmol/L glutamine. For IC₅₀ analyses TALL cell lines were plated in 96 well plates at 5000 cells/well, except for Tall-1 cells which were plated at 10,000 cells/well. Cells were re-fed with compound and media on day 4. Viability assays were performed using Cell Titer Glo kit (Promega, Cat. No. G7572, Fitchburg, Wis.) 7 days after compound addition. For larger scale compound treatments, TALL cell lines were plated in T-150 flasks at 200,000 cells/mL and treated at 0.1 or 1.0 μM GSI (MRK-003) for 3 days or as indicated. GSI washout studies were performed using 10 μM GSI. T-150 cultures were also re-fed on day 4.

Microarray Gene Expression Analysis: Total RNA isolated from cultured cells was used to make fluorescently labeled cRNA that was hybridized to DNA oligonucleotide microarrays as described previously (Marton, DeRisi et al. 1998; Hughes, Mao et al. 2001). Briefly, 4 μg of total RNA was used to synthesize dsDNA through RT. cRNA was produced by in vitro transcription and labeled post-synthetically with Cy3 or Cy5. Two populations of labeled cRNA, a reference population and an experimental population, were compared with each other by competitive hybridization to microarrays. Two hybridizations were done with each cRNA sample pair using a fluorescent dye reversal strategy. Human microarrays contained oligonucleotide probes corresponding to approximately 21,000 genes. All oligonucleotide probes on the microarrays were synthesized in situ with inkjet technology (Agilent Technologies, Palo Alto, Calif. (Hughes, Mao et al. 2001). After hybridization, arrays were scanned and fluorescence intensities for each probe were recorded. Ratios of transcript abundance (experimental to control) were obtained following normalization and correction of the array intensity data. Gene expression data analysis was done with the Rosetta Resolver gene expression. Expression levels (fold change) were generated by comparing each gene to the Stratagene Human Universal Reference (HURR) where untreated (baseline) cells were compared to IC50 for generation of Pearson correlation coefficients. In graphs where untreated (baseline) gene expression was binned into four GSI sensitivity groups (high, medium, low, none) the fold change of each gene (log ratio) was compared to the untreated average mRNA level for that gene(s) across all 16 cell lines. Log ratios from GSI treated cells were determined by comparing expression of each gene to a DMSO control prepared at the same time.

Flow Cytometry: DNA content analysis was performed using Propidium Iodide/RNase buffer (BD Biosciences, Cat. No. 550825, San Jose, Calif.) or Draq5. Briefly, 1×10(6) cells were harvested and fixed with 70% ethanol for 20 minutes on ice, washed and then resupended in 500 uL of PI buffer for 15 min at room temperature followed by analysis on flow cytometer (FACSCalibur, BD Biosciences). Draq5 staining was performed by incubating 200,000 live cells with 10 uM Draq5 for 5 minutes followed by flow cytometric analysis (488 nm excitation).

Quantitative PCR (qPCR):

T-ALL cell lines were treated with DMSO or MRK-003 (active GSI) for 48 hours and then harvested. RNA was isolated using the RNeasy Mini Kit (Qiagen, Cat#: 74106). cDNA was synthesized using High Capacity Archive Kit (Applied Biosystems, Cat #: 4368814). qPCR was performed on an ABI 7900 using ΔΔCT protocol using their inventoried Taqman Probes/Primers for human CDKN2D, CDKN1B and GAPDH (as internal control). Analysis was performed in SDS 2.2.2 software (Applied Biosystems).

Immunoblot: Standard western blotting procedures were used. Antibodies used as follows; Rb-underphosphorylated, (BD Biosciences, Cat. No 554164, San Jose, Calif.) Rb-Total (Cat. No. 9309), CDKN1B (Cell Signaling Technologies, Cat. No. 2552, Danvers, Mass.), CDKN2D (Santa Cruz Biotechnologies, Cat No. sc-1063, Santa Cruz, Calif.), Beta-Actin (Abeam Inc., Cat. No. 8226, Cambridge, Mass.). Immunoblot of NOTCH intracellular domain (NICD) was previously determined (O'Neil, Grim et al. 2007)

### Results

Identification of a NOTCH Pathway Gene Signature that Predicts Sensitivity to GSI.

Previous studies have identified mutations in the heterodimerization domain or PEST domain of the NOTCH1 gene in T-ALL cell lines and patient samples resulting in increased NICD and presumably activation of the NOTCH signaling pathway (Weng, Ferrando et al. 2004; Zhu, Zhao et al. 2006). In a subset of human T-ALL cell lines with activating mutations in NOTCH1, treatment with GSIs leads to cell cycle arrest and apoptosis (Weng, Ferrando et al. 2004; Lewis, Leveridge et al. 2007). We have previously shown that several cell lines with detectable levels of NICD are resistant to GSI treatment suggesting that levels of NICD are not a good predictor of GSI sensitivity (O'Neil, Grim et al. 2007). We determined IC₅₀ values for 16 T-ALL cell lines using a 7-day viability assay which allowed grouping into four sensitivity groups (1) high, IC₅₀<1 uM, (2) medium, IC₅₀ 3-4 uM, (3) low, IC₅₀ 6-7 uM, (4) none, IC₅₀>10 uM and comparison to NOTCH1 mutuation and NICD protein Levels (Table 1). These analyses demonstrate that neither NOTCH1 mutation status nor NICD protein levels predict sensitivity of T-ALL cell lines to GSI.

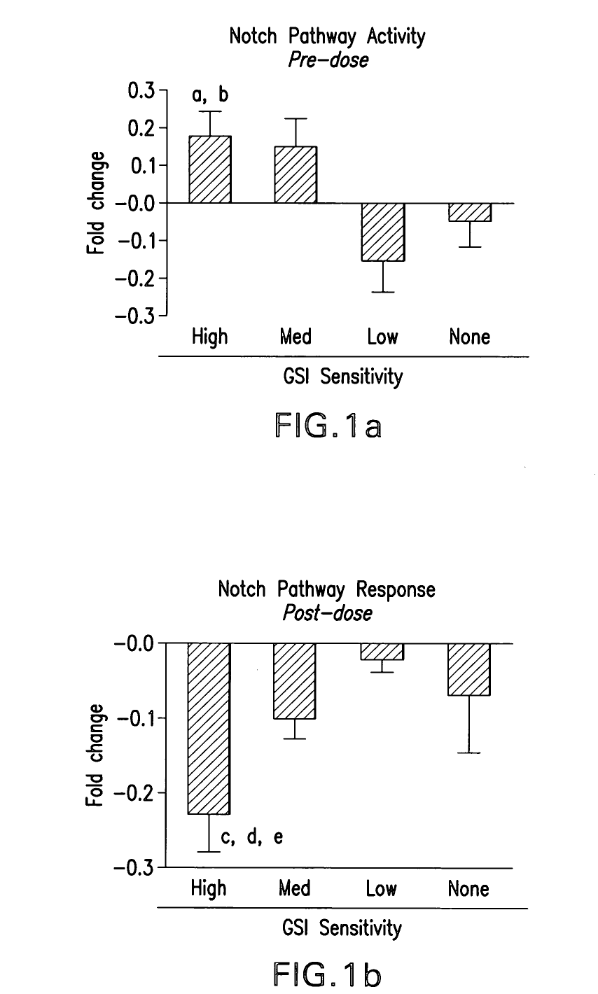

We used transcriptional profiling to determine if a gene signature could be identified that predicts sensitivity to GSI in T-ALL. The 16 T-ALL cell lines described in Table 1 were profiled and ten NOTCH target genes (HES-1, HES-4, HES-5, HEY-L, HEY-2, DTX1, C-MYC, NRARP, PTCRA, SHQ1) were used to assess NOTCH pathway activity (FIG. 1A). A composite expression score for NOTCH pathway activity was determined by calculating the average expression value of the ten NOTCH target genes (NOTCH-10) for each sensitivity group and comparing this to the overall average expression of these 10 genes across all 16 T-ALL cell lines. Indeed, the basal Notch pathway activity as measured with the NOTCH-10 signature correlates with sensitivity to GSI (FIG. 1A) Change in expression of the Notch-10 gene set in response to GSI treatment was also assessed We found that T-ALL cell lines with high sensitivity to GSI displayed a higher fold change in NOTCH target genes upon GSI treatment (FIG. 1B). Thus, we have identified a NOTCH gene signature that accurately predicts T-ALL cell lines with GSI sensitivity. In addition, we demonstrate that decrease in expression of this gene signature correlates and reports on the cytotoxic effects of the drug.

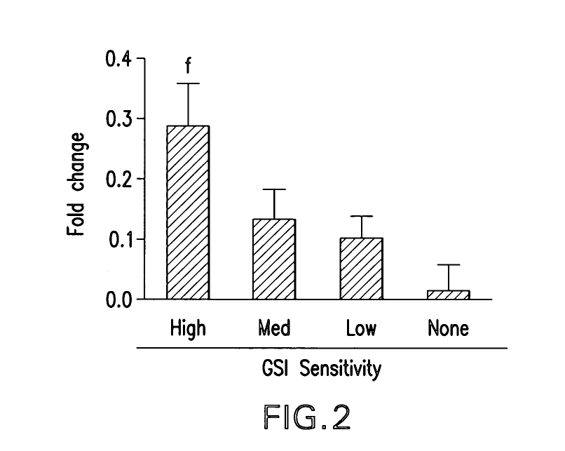

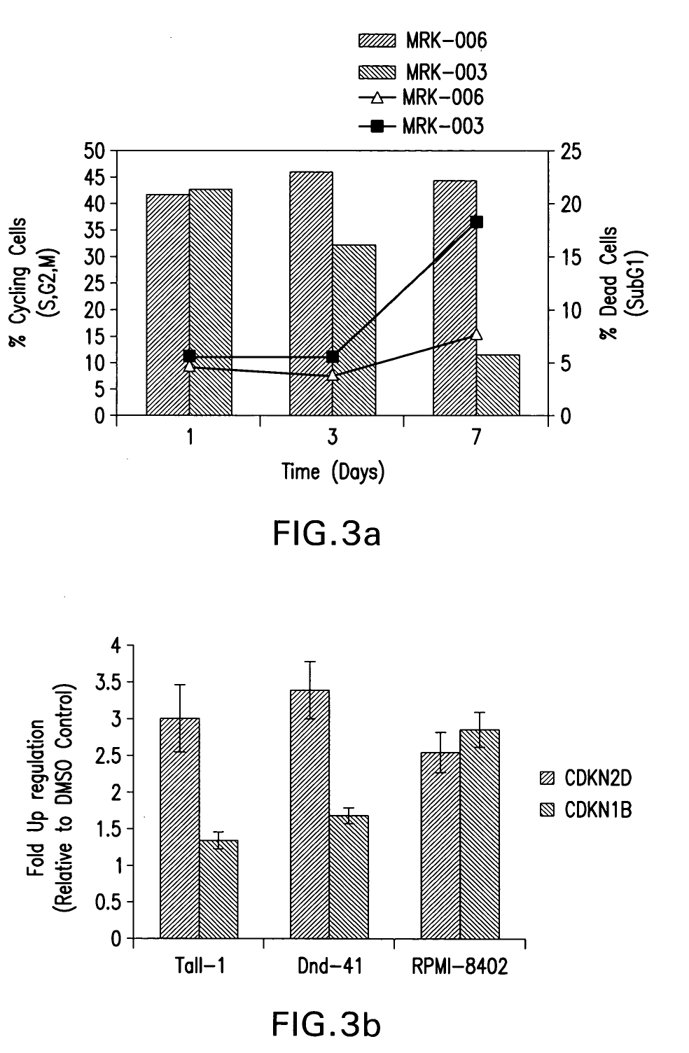

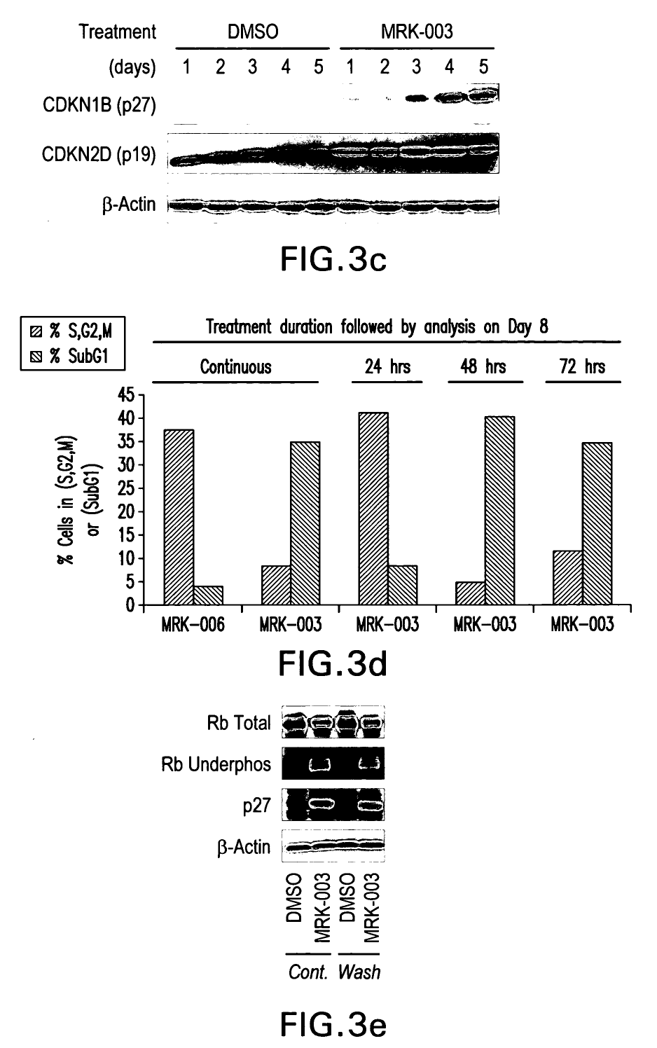

In addition to the Notch Pathway genes we evaluated changes in expression of several cyclin-dependent kinase inhibitors (CDKi), as cell cycle inhibition in G₀/G₁ is a hallmark of the GSI effect on T-ALL cells. We have found that the CDK genes, p19 (CDKN2D) & p27 (CDKN1B) are induced in T-ALL cells in response to GSI. Expression of these genes alone or combined after exposure to GSI, significantly correlates with sensitivity across the panel of T-ALL cell lines (FIG. 2). GSI induced cell cycle arrest (FIG. 3A) and subsequent increases in the CDKi CDKN2D and CDKN1B are also evident by quantitative PCR and immunblot (FIGS. 3B and 3C respectively) in a representative GSI sensitive cell line TALL1. These effects were only observed with the active GSI MRK-003, while the 275-fold leas active enantiomer MRK-006, used as a control, showed no effect.

Thus, we have identified a NOTCH gene signature that accurately predicts T-ALL cell lines with GSI sensitivity. In addition, we demonstrate that decrease in the expression of the Notch signature and increase in CDKi gene signature correlate and report on the cytotoxic effects of the GSI.

Still smaller Notch target genes sets and individual genes may also be useful in evaluating Notch pathway activity. Expression of individual genes and composites shown of genes in Table 2 were correlated to IC₅₀'s using a Pearson two-tailed correlation analysis. Individual genes or composite scores which correlate (p<0.05) with IC₅₀ are summarized in Table 3 and correlation analysis is shown in Table 4. Using this method both the Notch-10 composite score, a HES1-MYC composite score, as well as DTX1, correlate to GSI sensitivity.

In an effort to optimally analyze the Notch dependent genes we next removed one of the sixteen cell lines, KARPAS-45, from the correlation analysis, based on the fact it contains a MLL-AFX fusion. Cells containing MLL fusions have been reported represent a unique sub-type of T-ALL and contain down regulated levels of cell cycle genes relative to other T-ALL cells (Ferrando, Armstrong et al. 2003). Reanalysis in the absence of the KARPAS-45 cell line determined that HES1, HES5 mRNA levels now correlate to GSI-Sensitivity, in addition to the genes previously identified and describe in Table 3, (correlation analysis shown in Table 5). Similar correlation analysis was conducted using GSI treated cells. This identified the NOTCH target genes (HES5, DTX1, HES4, MYC, SHQ1, Notch-10 composite) as well as cell cycle genes (p19, p27 and p21) as markers of target inhibition and response. The use of both individual genes and composite scores may be useful in identifying Notch activated cells and tumors in preclinical and clinical settings as well as demonstrating the effect of NOTCH pathway inhibitors. In addition to benefiting patient stratification and demonstrating NOTCH pathway inhibitors effect in T-ALL patients, such markers can be applied to other NOTCH dependent tumors i.e. cervical, head and neck, endometrial, renal, lung, pancreatic, breast, osteosarcoma, mesothelioma, glioma, medulloblastoma, other hematologic malignancies and other NOTCH driven diseases (reviewed in (Miele, Miao et al. 2006)).

### Example 2

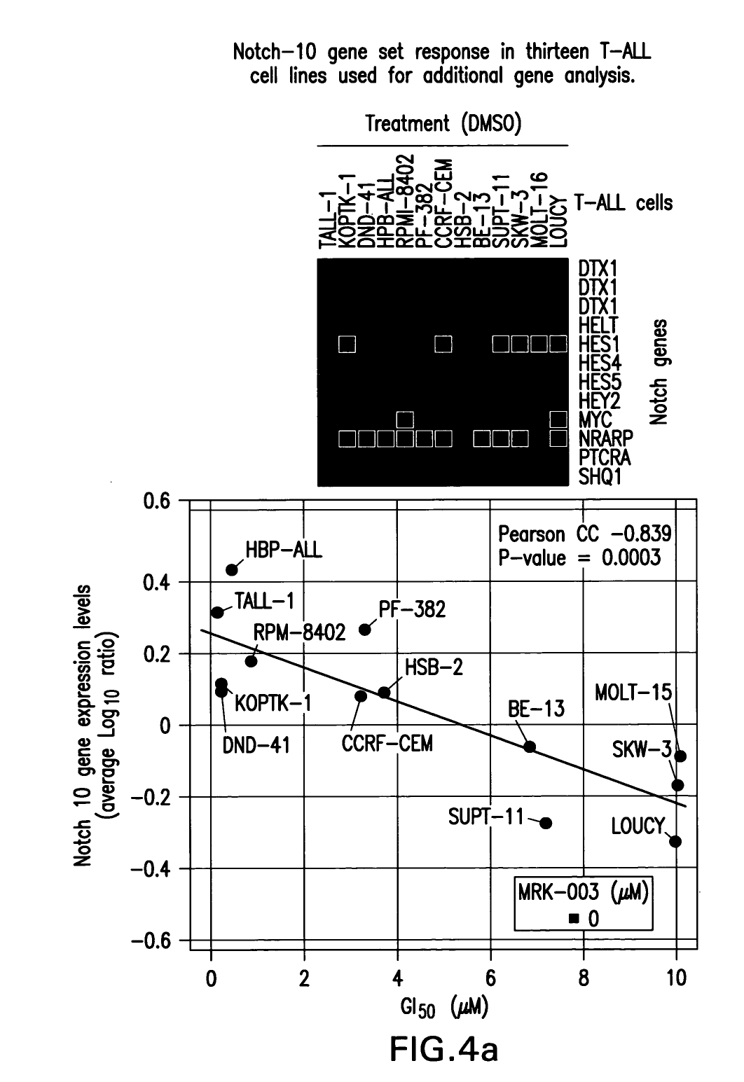

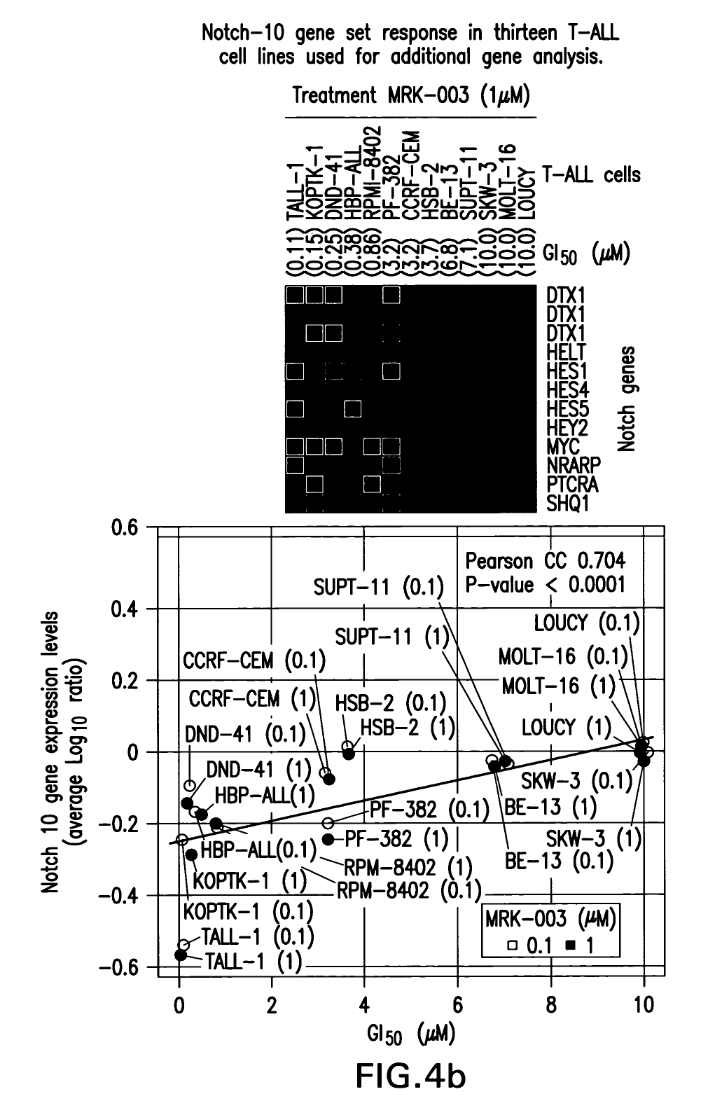

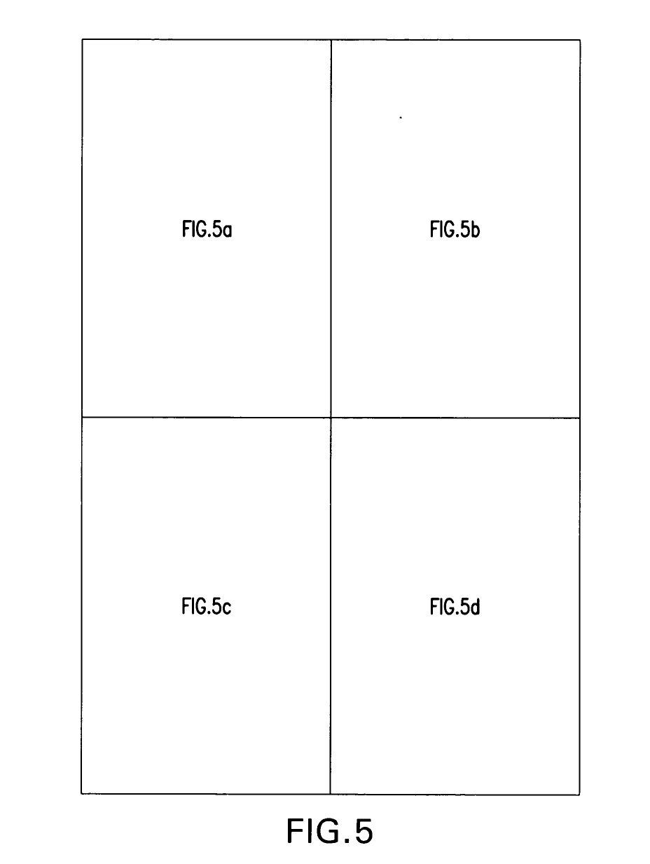

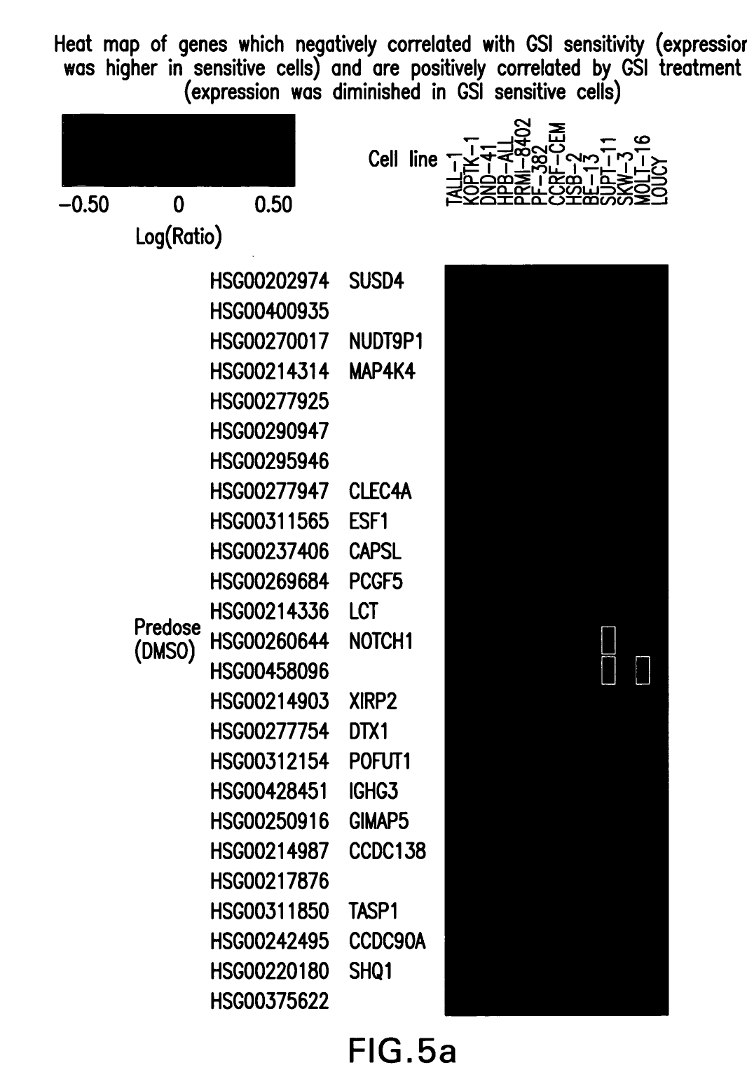

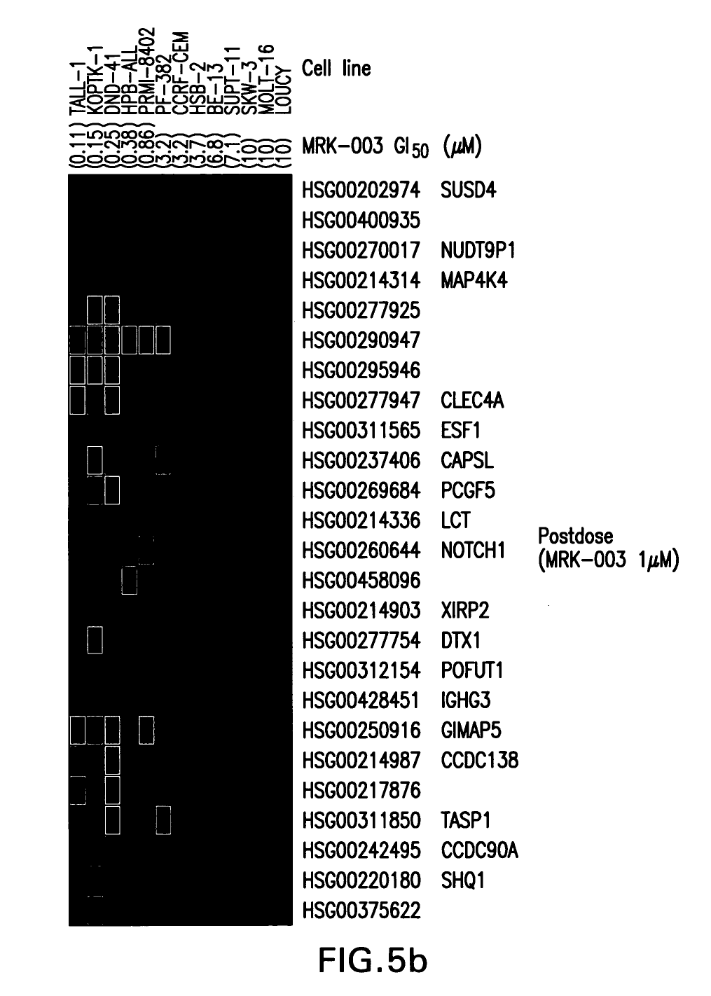

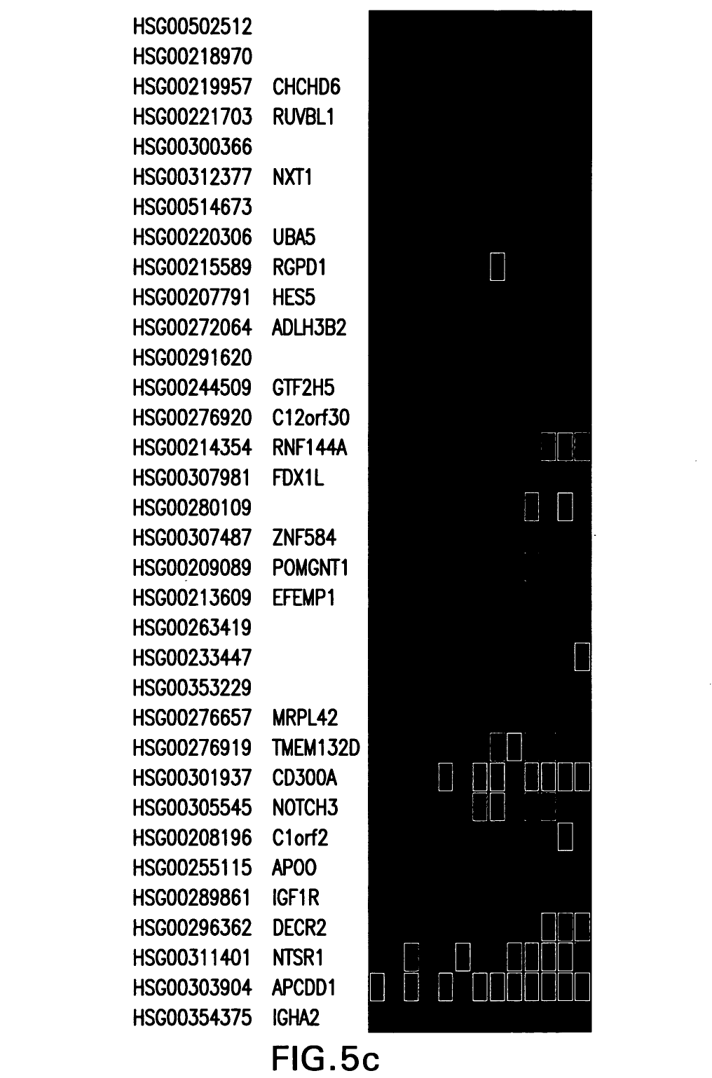

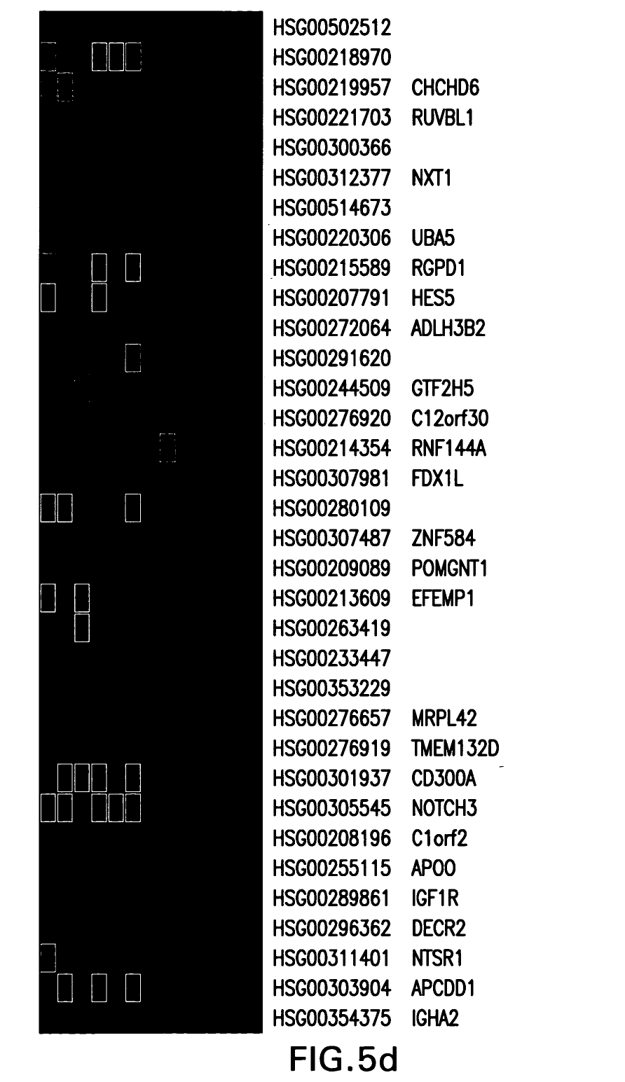

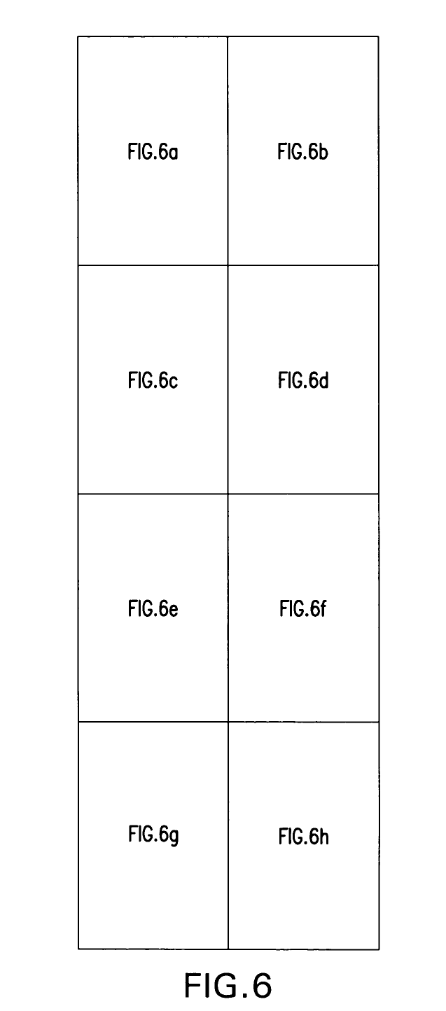

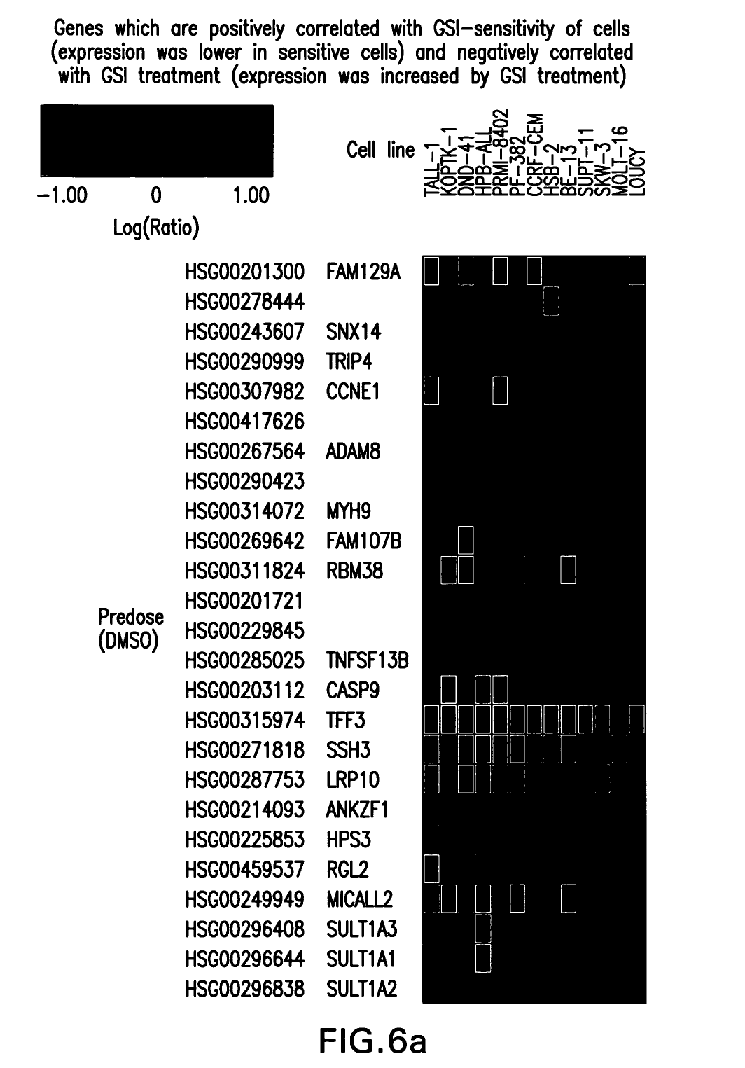

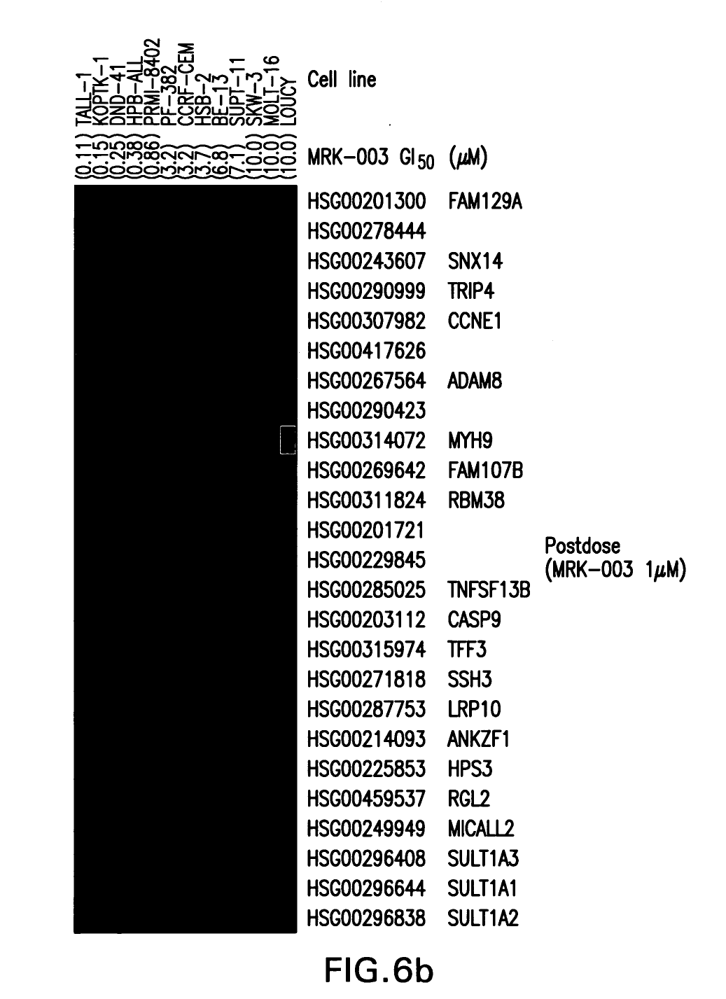

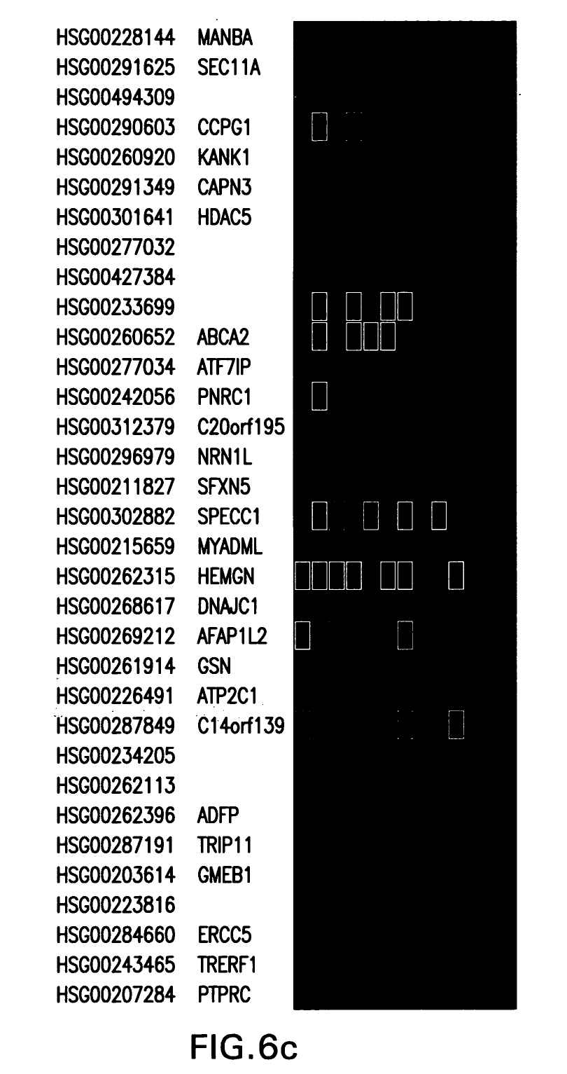

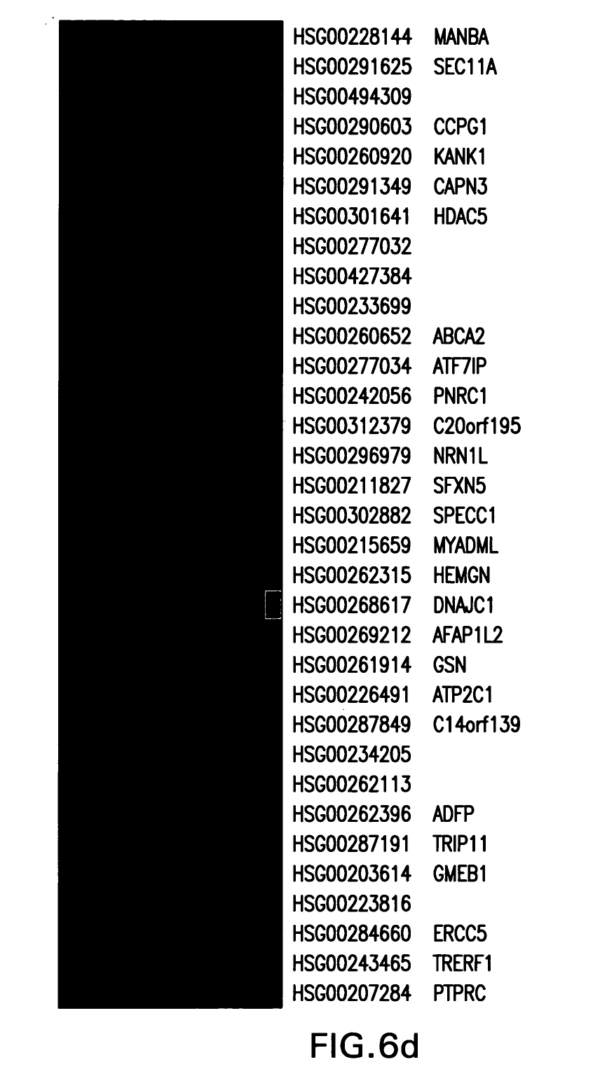

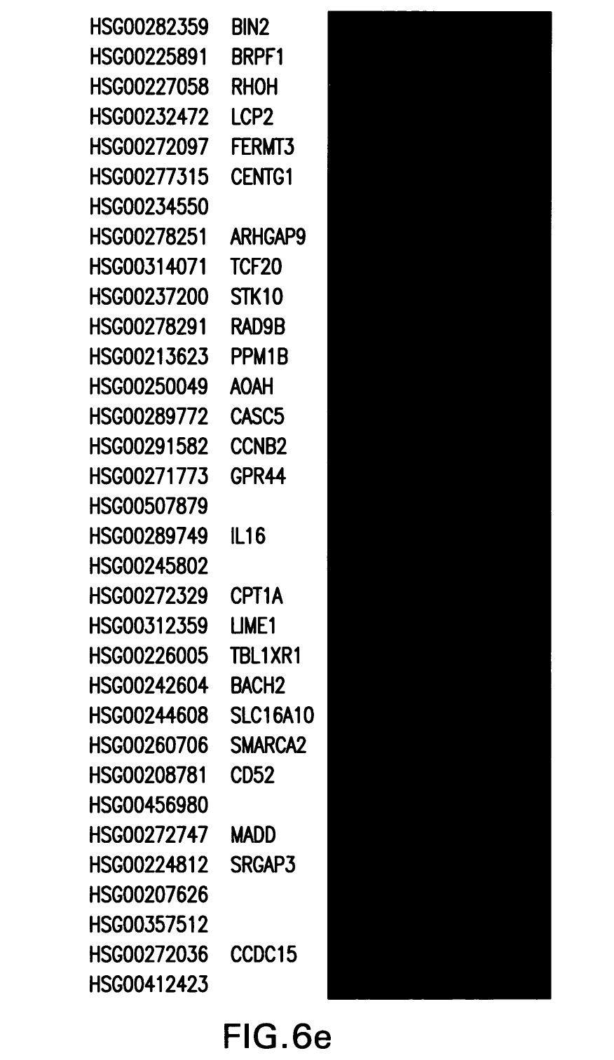

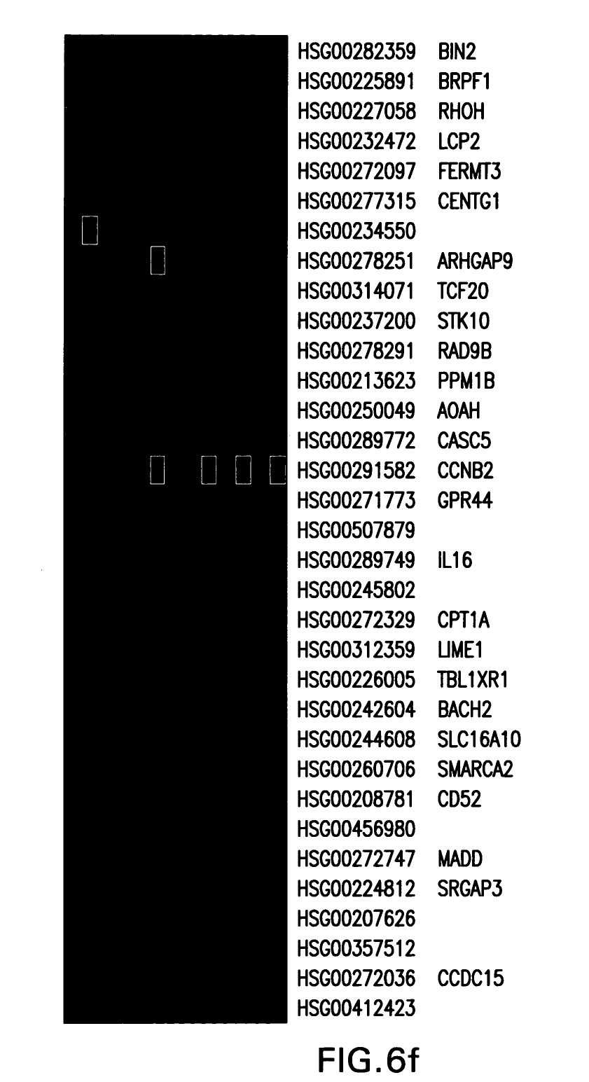

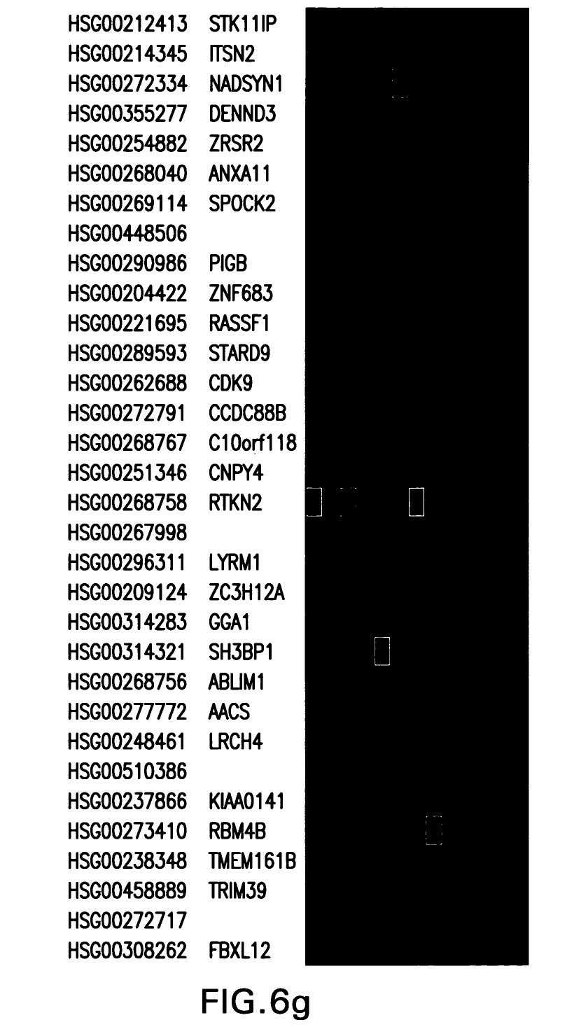

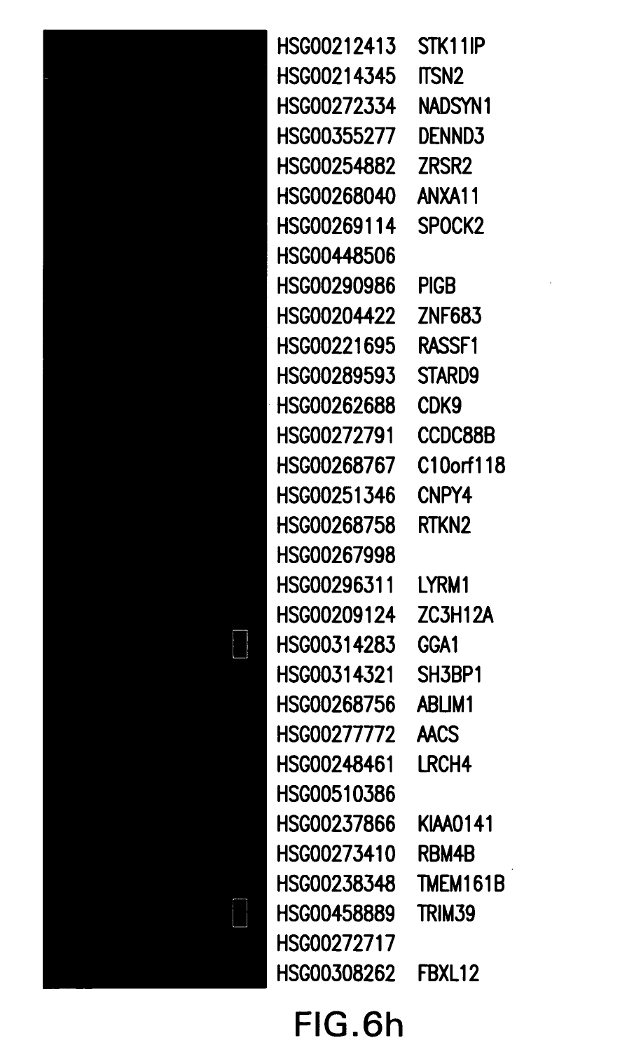

To identify additional predictive and response genes we utilized mRNA expression data from thirteen T-ALL cell lines where the Notch-10 gene set score showed good correlation with GSI-sensitivity in both DMSO control and MRK-003 treated cells (FIG. 4). Sixty three genes were identified which positively correlated with GSI-sensitivity predose (higher in GSI-sensitive cells predose) (correlation coefficient <−0.4, p<0.05) and whose expression was diminished upon GSI-treatment (MRK-003) (correlation coefficient >0.04, p<0.05), (Table 7 and FIG. 5). These genes likely to include Notch target genes and includes three genes associated with the Notch-10 gene set (DTX1, SHQ1, HES5), two other Notch target genes NOTCH3 and TASP1 (Palomero, T., M. L. Sulis et al. (2006) “NOTCH1 directly regulates c-MYC and activates a feed-forward-loop transcriptional network promoting leukemic cell growth.” Proc Natl Acad Sci USA 103(48):18261-6), as well as NOTCH1 itself. One hundred and thirty one genes were identified to be anti-correlated with GSI-sensitivity predose (lower expression in GSI-sensitive cells predose) (correlation coefficient >0.4, p<0.05) and upon GSI-treatment (MRK-003) were up-regulated (correlation coefficient >0.04, p<0.05) (Table 8 and FIG. 6). Together or individually these 194 genes represent the most robust set of genes which predict GSI-sensitivity and are also capable of demonstrating GSI response.

### REFERENCES

Artavanis-Tsakonas, S., M. D. Rand, et al. (1999). “Notch signaling: cell fate control and signal integration in development.” Science 284(5415): 770-6.
Ellisen, L. W., J. Bird, et al. (1991). “TAN-1, the human homolog of the Drosophila notch gene, is broken by chromosomal translocations in T lymphoblastic neoplasms.” Cell 66(4): 649-61.
Grabher, C., H. von Boehmer, et al. (2006). “Notch 1 activation in the molecular pathogenesis of T-cell acute lymphoblastic leukaemia.” Nat Rev Cancer 6(5): 347-59.
Hughes, T. R., M. Mao, et al. (2001). “Expression profiling using microarrays fabricated by an ink-jet oligonucleotide synthesizer.” Nat Biotechnol 19(4): 342-7.
Lewis, H. D., M. Leveridge, et al. (2007). “Apoptosis in T cell acute lymphoblastic leukemia cells after cell cycle arrest induced by pharmacological inhibition of notch signaling.” Chem Biol 14(2): 209-19.
Marton, M. J., J. L. DeRisi, et al. (1998). “Drug target validation and identification of secondary drug target effects using DNA microarrays.” Nat Med 4(11): 1293-301.
Miele, L., H. Miao, et al. (2006). “NOTCH signaling as a novel cancer therapeutic target.” Curr Cancer Drug Targets 6(4): 313-23.
Mumm, J. S, and R. Kopan (2000). “Notch signaling: from the outside in.” Dev Biol 228(2): 151-65.
O'Neil, J., J. Grim, et al. (2007). “FBW7 mutations in leukemic cells mediate NOTCH pathway activation and resistance to {gamma}-secretase inhibitors.” J. Exp. Med.: jem.20070876.
Pear, W. S., J. C. Aster, et al. (1996). “Exclusive development of T cell neoplasms in mice transplanted with bone marrow expressing activated Notch alleles.” J Exp Med 183(5): 2283-91.
Reynolds, T. C., S. D. Smith, et al. (1987). “Analysis of DNA surrounding the breakpoints of chromosomal translocations involving the beta T cell receptor gene in human lymphoblastic neoplasms.” Cell 50(1): 107-17.
Weng, A. P., A. A. Ferrando, et al. (2004). “Activating mutations of NOTCH1 in human T cell acute lymphoblastic leukemia.” Science 306(5694): 269-71.
Zhu, Y. M., W. L. Zhao, et al. (2006). “NOTCH1 mutations in T-cell acute lymphoblastic leukemia: prognostic significance and implication in multifactorial leukemogenesis.” Clin Cancer Res 12(10): 3043-9.

TABLE 1
Sensitivity of human T-ALL cell lines to
the gamma-secretase inhibitor MRK-003

| | | | IC₅₀ | GSI | Group |
|---|---|---|---|---|---|
| Cell Line | Notch-1 | NICD | (uM) | Sensitivity | Size |
| TALL-1 | Wt | 0 | 0.11 | High | 37.5% |
| KOPTK-1 | Mutated | 3 | 0.15 | | |
| DND41 | Mutated | 2 | 0.25 | | |
| HBP-ALL | Mutated | 3 | 0.38 | | |
| KARPAS-45 | Mutated | 0 | 0.81 | | |
| RPMI 8402 | Mutated | 3 | 0.86 | | |
| CEM | Mutated | 2 | 3.2 | Medium | 18.75% |
| PF-382 | Mutated | 3 | 3.2 | | |
| HSB-2 | Wt | 1 | 3.7 | | |
| BE-13 | Mutated | 3 | 6.8 | Low | 12.5% |
| SUPT-11 | Wt | 0 | 7.1 | | |
| JURKAT | Wt | 3 | >10 | None | 31.25% |
| LOUCY | Wt | 0 | >10 | | |
| MOLT -4 | Mutated | 3 | >10 | | |
| MOLT-16 | Wt | 0 | >10 | | |
| SKW-3 | Mutated | 1 | >10 | | |

NICD levels (immunoblot): 0 = None, 1 = Low, 2 = Medium, 3 = High

GSI IC₅₀s, based on a 7-day ATP viability assay, are summarized for 16 T-ALL cell lines and grouped into high (>1 uM), medium (3-4 uM), low (6-7 uM) or insensitive groupings (>10 uM). Also summarized is NOTCH1 mutation status, NICD levels and cell line group size for each sensitivity level previously described (O'Neil et al. 2007). NICD levels are reported qualitatively based on immunoblot signal, 0=none, 1=low, 2=medium, 3=high.

TABLE 2
Marker gene identifiers and accession numbers

| | | | Gene/Gene | | |
|---|---|---|---|---|---|
| Input | Gene Model | Transcript | Symbol | Gene/Name | Gene/Alternate Gene Symbol |
| HES1 | HSG00222224 | NM_005524 | HES1 | hairy and enhancer of split 1, (Drosophila) | FLJ20408; HES-1; Hes1; HHL; |
| | | | | | HRY; (HHL) |
| CDKN1B | HSG00282095 | NM_004064 | CDKN1B | cyclin-dependent kinase inhibitor 1B (p27, Kip1) | KIP1; MEN1B; P27KIP1; CDKN4 |
| DTX1 | HSG00277754 | NM_004416 | DTX1 | deltex homolog 1 (Drosophila) | hDx-1 |
| CDKN2D | HSG00306660 | NM_079421 | CDKN2D | cyclin-dependent kinase inhibitor 2D (p19, | INK4D; p19; p19-INK4D; |
| | | | | inhibits CDK4) | p19INK4d; (P19) |
| HES5 | HSG00207791 | NM_001010926 | HES5 | hairy and enhancer of split 5 (Drosophila) | LOC256482 |
| PTCRA | HSG00243539 | NM_138296 | PTCRA | pre T-cell antigen receptor alpha | PT-ALPHA; PTA; Hs.169002 |
| HEY2 | HSG00244588 | NM_012259 | HEY2 | hairy/enhancer-of-split related with | CHF1; GRL; HERP1; HESR2; |
| | | | | YRPW motif 2 | HRT2; MGC10720; (GRL) |
| HESL | HSG00228923 | NM_001029887 | HEYL | Heslike; Hey-like transcriptional repressor | HELT; HESL; Mgn; HCM1228 |
| CDKN1A | HSG00242011 | NM_000389 | CDKN1A | cyclin-dependent kinase inhibitor 1A (p21, Cip1) | (P21); CAP20; CDKN1; CIP1; |
| | | | | | MDA-6; P21; p21-Cip1; p21CIP1; |
| | | | | | p21WAF1; SDI1; WAF1; (CIP1) |
| SHQ1 | HSG00220180 | NM_018130 | SHQ1 | SHQ1 homolog (S. cerevisiae) | FLJ10539; DKFZp686H07226 |
| MYC | HSG00258060 | NM_002467 | MYC | v-myc myelocytomatosis viral oncogene | c-Myc |
| | | | | homolog (avian) | |
| Hes4 | HSG00317508 | NM_021170 | HES4 | hairy and enhancer of split 4 (Drosophila) | LOC57801; Hes4 |
| NRARP | HSG00351624 | NM_001004354 | NRARP | similar to ankyrin-repeat protein Nrarp | MGC61598 |

TABLE 3
Summary of mRNA levels which predict sensitivity of T-ALL
cells to GSIs and demonstrate target inhibition or response

| | | | | Demon- |
|---|---|---|---|---|
| | # Genes | | | strates |
| Gene Type | in Set | Gene | Predictive | Effect |
| Notch | 1 | HES1 * | Yes | — |
| Targets | 2 | HES1, MYC | Yes | — |
| | 1 | HES5 * | Yes | Yes |
| | 1 | DTX1 | Yes | Yes |
| | 10 | HES1, -4, -5, L, | Yes | Yes |
| | | HEY-2, DTX1, MYC, | | |
| | | NRARP, PTCRA, SHQ1 | | |
| | 1 | HES4 | — | Yes |
| | 1 | MYC | — | Yes |
| | 1 | SHQ1 | — | Yes |
| Cell Cycle | 1 | p19 | — | Yes |
| | 1 | p21 | — | Yes |
| | 1 | p27 | — | Yes |

Correlation coefficients and p-values for mRNA levels vs. GSI sensitivity can be found in Tables 4 and 5. *See Table 5

TABLE 4
Correlation coefficients (CC) and p-values of genes
whose levels predict GSI sensitivity or
demonstrate GSI effect

| | | | | Predictive | | | | | |
|---|---|---|---|---|---|---|---|---|---|
| | | | | of GSI- | | Demonstrate | | | |
| | | | | Baseline | | GSI Effect | | | |
| | | | | vs. HUR | | 0.1 uM, day 3 | | 1 uM, day 3 | |
| Mechansim | # genes | Name | Gene Symbol | CC | p-value | CC | p-value | CC | p-value |
| Notch | 1 | Hes1 | HES1 | −0.47 | 0.064 | 0.18 | 0.521 | 0.06 | 0.828 |
| | 2 | HES1-MYC | HES1, MYC | −0.54 | 0.029 | 0.44 | 0.090 | 0.41 | 0.113 |
| | 1 | HES5 | HES5 | −0.49 | 0.056 | 0.45 | 0.079 | 0.49 | 0.056 |
| | 1 | DTX1 | DTX1 | −0.51 | 0.045 | 0.55 | 0.028 | 0.52 | 0.037 |
| | 10 | 10 gene Notch set | HES1, HES4, HES5, | −0.56 | 0.023 | 0.52 | 0.038 | 0.49 | 0.055 |
| | | | HEY2, HESL, DTX1, | | | | | | |
| | | | MYC, NRARP-related, | | | | | | |
| | | | PTCRA, SHQ1 | | | | | | |
| | 1 | MYC | MYC | −0.30 | 0.267 | 0.59 | 0.017 | 0.60 | 0.015 |
| | 1 | SHQ1 | SHQ1 | −0.31 | 0.253 | 0.60 | 0.014 | 0.47 | 0.066 |
| | 1 | HES4 | HES4 | −0.33 | 0.209 | 0.41 | 0.119 | 0.41 | 0.119 |
| | 1 | NRARP | NRARP-related | −0.27 | 0.318 | 0.24 | 0.362 | 0.34 | 0.193 |
| | 1 | PTCRA | PTCRA | −0.18 | 0.499 | 0.09 | 0.746 | 0.01 | 0.960 |
| | 1 | HEY2 | HEY2 | 0.31 | 0.247 | −0.01 | 0.958 | −0.23 | 0.390 |
| | 1 | HEY-like | HESL | 0.30 | 0.258 | −0.01 | 0.977 | 0.10 | 0.724 |
| Cell Cycle | 1 | p19 | CDKN2D | 0.38 | 0.150 | −0.65 | 0.007 | −0.53 | 0.035 |
| | 1 | p21 | CDKN1A | −0.03 | 0.900 | 0.46 | 0.070 | 0.30 | 0.263 |
| | 1 | p27 | CDKN1B | −0.22 | 0.422 | −0.42 | 0.104 | −0.68 | 0.006 |

TABLE 5
Correlation coefficients (CC) and p-values of genes
whose levels predict GSI sensitivity or demonstrate GSI effect
(* KARPAS-45 cell line data removed)

| | | | | Predictive | | | | | |
|---|---|---|---|---|---|---|---|---|---|
| | | | | of GSI- | | | | | |
| | | | | Sensitivity | | Demonstrate GSI | | | |
| | | | | (p < 0.05) | | Effect (p < 0.05) | | | |
| | | | | Baseline | | 0.1 uM, | | 1 uM, | |
| | | | | vs. HUR | | day 3 | | day 3 | |
| Mechansim | # genes | Name | Gene Symbol | CC | p-value | CC | p-value | CC | p-value |
| Notch | 1 | Hes1 | HES1 | −0.55 | 0.036 | 0.22 | 0.455 | 0.10 | 0.742 |
| | 2 | HES1-MYC | HES1, MYC | −0.56 | 0.031 | 0.48 | 0.071 | 0.49 | 0.064 |
| | 1 | HES5 | HES5 | −0.54 | 0.038 | 0.53 | 0.042 | 0.54 | 0.038 |
| | 1 | DTX1 | DTX1 | −0.60 | 0.018 | 0.64 | 0.010 | 0.61 | 0.015 |
| | 10 | 10 gene Notch | HES1, HES4, HES5, | −0.64 | 0.010 | 0.62 | 0.014 | 0.58 | 0.023 |
| | | | HEY2, HESL, DTX1, | | | | | | |
| | | | MYC, NRARP-related, | | | | | | |
| | | | PTCRA, SHQ1 | | | | | | |
| | 1 | MYC | MYC | −0.25 | 0.368 | 0.62 | 0.014 | 0.69 | 0.004 |
| | 1 | SHQ1 | SHQ1 | −0.31 | 0.253 | 0.69 | 0.004 | 0.64 | 0.010 |
| | 1 | HES4 | HES4 | −0.48 | 0.072 | 0.60 | 0.018 | 0.45 | 0.096 |
| | 1 | NRARP | NRARP-related | −0.31 | 0.265 | 0.28 | 0.321 | 0.37 | 0.170 |
| | 1 | PTCRA | PTCRA | −0.22 | 0.427 | 0.14 | 0.622 | 0.06 | 0.845 |
| | 1 | HEY2 | HEY2 | 0.39 | 0.154 | 0.04 | 0.875 | −0.21 | 0.444 |
| | 1 | HEY-like | HESL | 0.26 | 0.354 | 0.08 | 0.768 | 0.02 | 0.953 |
| Cell Cycle | 1 | p19 | CDKN2D | 0.37 | 0.179 | −0.77 | 0.001 | −0.68 | 0.005 |
| | 1 | p21 | CDKN1A | −0.07 | 0.812 | 0.52 | 0.045 | 0.33 | 0.226 |
| | 1 | p27 | CDKN1B | −0.26 | 0.349 | −0.62 | 0.013 | −0.70 | 0.005 |

TABLE 6
Genes which negatively correlated with GSI sensitivity (expression was higher in sensitive
cells) and are positively correlated by GSI treatment (expression was diminished in GSI sensitive
cells)

| | | | | | GI50 | | GI50 | |
|---|---|---|---|---|---|---|---|---|
| Gene | Gene | | | Alternate Gene | Correlation | p-value | Correlation | p-value |
| Symbol | Model | Transcript | Gene/Name | Symbol | Predose | Predose | Postdose | Postdose |
| NaN | HSG00514673 | 10025931768 | | | −0.556 | 0.050 | 0.506 | 0.009 |
| CLEC4A | HSG00277947 | 10023808951 | C-type lectin domain | DCIR; DDB27; | −0.557 | 0.050 | 0.510 | 0.008 |
| | | | family 4, member A | HDCGC13P; | | | | |
| | | | | LLIR; CLECSF6 | | | | |
| GTF2H5 | HSG00244509 | 10025916728 | general transcription | (TTD); | −0.557 | 0.049 | 0.430 | 0.028 |
| | | | factor IIH, | bA120J8.2; | | | | |
| | | | polypeptide 5 | C6orf175; TFB5; | | | | |
| | | | | TGF2H5; TTD; | | | | |
| | | | | TTD-A; TTDA; | | | | |
| | | | | (FLJ30544) | | | | |
| APCDD1 | HSG00303904 | 10025910447 | adenomatosis | DRAPC1; | −0.558 | 0.049 | 0.439 | 0.025 |
| | | | polyposis coli down- | FP7019; B7323 | | | | |
| | | | regulated 1 | | | | | |
| POFUT1 | HSG00312154 | 10025904701 | protein O- | KIAA0180; | −0.558 | 0.049 | 0.416 | 0.033 |
| | | | fucosyltransferase 1 | MGC2482; O- | | | | |
| | | | | Fuc-T; O-FucT- | | | | |
| | | | | 1; O-FUT; | | | | |
| | | | | FUT12 | | | | |
| HES5 | HSG00207791 | 10025921292 | hairy and enhancer | LOC256482 | −0.560 | 0.048 | 0.516 | 0.007 |
| | | | of split 5 | | | | | |
| | | | (Drosophila) | | | | | |
| RGPD1 | HSG00215589 | 10025924124 | RANBP2-like and | LOC388974; | −0.569 | 0.044 | 0.509 | 0.008 |
| | | | GRIP domain | LOC400966; | | | | |
| | | | containing 1 | RGP1; | | | | |
| | | | | LOC339749 | | | | |
| RNF144 | HSG00214354 | 10023813723 | | | −0.570 | 0.043 | 0.574 | 0.002 |
| NaN | HSG00502512 | 10025923080 | | | −0.570 | 0.043 | 0.470 | 0.015 |
| SHQ1 | HSG00220180 | 10025909122 | SHQ1 homolog | FLJ10539; | −0.573 | 0.042 | 0.750 | 0.000016 |
| | | | (S. cerevisiae) | DKFZp686H07226 | | | | |
| IGHG3 | HSG00428451 | 10023823209 | immunoglobulin | FLJ39988; | −0.575 | 0.041 | 0.511 | 0.008 |
| | | | heavy constant | FLJ40036; | | | | |
| | | | gamma 3 (G3m | FLJ40253; | | | | |
| | | | marker) | FLJ40587; | | | | |
| | | | | FLJ40789; | | | | |
| | | | | FLJ40834; | | | | |
| | | | | MGC45809; | | | | |
| | | | | DKFZp686H11213 | | | | |
| UBE1DC1 | HSG00220306 | 10025905958 | ubiquitin-activating | FLJ23251Uba5; | −0.577 | 0.041 | 0.478 | 0.014 |
| | | | enzyme E1-domain | Uba5; FLJ23251 | | | | |
| | | | containing 1 | | | | | |
| NTSR1 | HSG00311401 | 10025902150 | neurotensin receptor | hNTR; NT1; | −0.577 | 0.041 | 0.440 | 0.024 |
| | | | 1 (high affinity) | NT1-R; NTR; | | | | |
| | | | | Ntr1; NTSRH; | | | | |
| | | | | hNT1-R | | | | |
| ZNF584 | HSG00307487 | 10025906095 | zinc finger protein | LOC201514; | −0.577 | 0.040 | 0.610 | 0.001 |
| | | | 584 | FLJ39899 | | | | |
| NaN | HSG00289861 | 10025907002 | | | −0.579 | 0.040 | 0.579 | 0.002 |
| POMGNT1 | HSG00209089 | 10025907173 | protein O-linked | FLJ20277; | −0.579 | 0.040 | 0.434 | 0.026 |
| | | | mannose beta1,2-N- | GnTI.2; MEB; | | | | |
| | | | acetylglucosaminyltransferase | MGAT1.2; | | | | |
| | | | | POMGnT1; | | | | |
| | | | | UDP-GlcNAc; | | | | |
| | | | | DKFZp761B182 | | | | |
| GIMAP5 | HSG00250916 | 10023811284 | GTPase, IMAP | hIAN5; HIMAP3; | −0.581 | 0.039 | 0.782 | 0.000004 |
| | | | family member 5 | IAN-5; IAN4; | | | | |
| | | | | IAN4L1; IAN5; | | | | |
| | | | | IMAP3; | | | | |
| | | | | FLJ11296 | | | | |
| MRPL42 | HSG00276657 | 10025910717 | mitochondrial | MRP-L31; | −0.585 | 0.037 | 0.434 | 0.026 |
| | | | ribosomal protein | MRPS32; | | | | |
| | | | L42 | PTD007; | | | | |
| | | | | RPML31; | | | | |
| | | | | HSPC204 | | | | |
| NUDT9P1 | HSG00270017 | 10025908138 | nudix (nucleoside | C10orf98; | −0.585 | 0.037 | 0.400 | 0.041 |
| | | | diphosphate linked | MGC34007; | | | | |
| | | | moiety X)-type motif | bA56M3.1 | | | | |
| | | | 9 pseudogene 1 | | | | | |
| LCT | HSG00214336 | 10025905326 | lactase | LAC; LPH; | −0.586 | 0.037 | 0.467 | 0.016 |
| | | | | LPH1; (LPH) | | | | |
| NaN | HSG00280109 | 10025929162 | | | −0.586 | 0.037 | 0.592 | 0.002 |
| NaN | HSG00458096 | 10025930675 | | | −0.589 | 0.036 | 0.634 | 0.001 |
| NaN | HSG00291620 | 10023816194 | | | −0.592 | 0.035 | 0.425 | 0.030 |
| DECR2 | HSG00296362 | 10023810624 | 2,4-dienoyl CoA | PDCR | −0.594 | 0.034 | 0.541 | 0.005 |
| | | | reductase 2, | | | | | |
| | | | peroxisomal | | | | | |
| NaN | HSG00353229 | 10025930613 | | | −0.595 | 0.034 | 0.536 | 0.005 |
| NaN | HSG00218970 | 10025914636 | | | −0.596 | 0.033 | 0.603 | 0.001 |
| NaN | HSG00263419 | 10025918838 | | | −0.596 | 0.033 | 0.642 | 0.001 |
| IGHA2 | HSG00354375 | 10023828965 | immunoglobulin heavy constant alpha 2 | | −0.597 | 0.033 | 0.524 | 0.006 |
| | | | (A2m marker) | | | | | |
| CAPSL | HSG00237406 | 10025910211 | calcyphosine-like | MGC26610 | −0.599 | 0.032 | 0.613 | 0.001 |
| IGHA2 | HSG00354375 | 10023843004 | immunoglobulin heavy constant alpha 2 | | −0.601 | 0.032 | 0.637 | 0.001 |
| | | | (A2m marker) | | | | | |
| NOTCH1 | HSG00260644 | 10025910795 | Notch homolog 1, | hN1; NIC; TAN1; | −0.602 | 0.031 | 0.406 | 0.038 |
| | | | translocation- | FLJ20005 | | | | |
| | | | associated | | | | | |
| | | | (Drosophila) | | | | | |
| TMEM132D | HSG00276919 | 10023828344 | transmembrane | MGC138770; | −0.608 | 0.029 | 0.423 | 0.030 |
| | | | protein 132D | MGC138771; | | | | |
| | | | | MOLT; | | | | |
| | | | | KIAA1944 | | | | |
| CMYA3 | HSG00214903 | 10025906812 | | | −0.610 | 0.028 | 0.415 | 0.034 |
| PCGF5 | HSG00269684 | 10025906395 | polycomb group ring | MGC16202; | −0.613 | 0.027 | 0.551 | 0.004 |
| | | | finger 5 | RNF159; | | | | |
| | | | | Hs.246914 | | | | |
| MRPL42 | HSG00276657 | 10023809605 | mitochondrial | MRP-L31; | −0.617 | 0.026 | 0.740 | 0.000024 |
| | | | ribosomal protein | MRPS32; | | | | |
| | | | L42 | PTD007; | | | | |
| | | | | RPML31; | | | | |
| | | | | HSPC204 | | | | |
| ALDH3B2 | HSG00272064 | 10023809943 | aldehyde | Hs.87539; | −0.618 | 0.026 | 0.799 | 0.000002 |
| | | | dehydrogenase 3 | ALDH8 | | | | |
| | | | family, member B2 | | | | | |
| NaN | HSG00307981 | 10025906387 | | | −0.620 | 0.025 | 0.547 | 0.004 |
| NaN | HSG00233447 | 10025927023 | | | −0.622 | 0.025 | 0.448 | 0.022 |
| NXT1 | HSG00312377 | 10025902738 | NTF2-like export | MTR2; P15; | −0.635 | 0.021 | 0.490 | 0.011 |
| | | | factor 1 | (P15) | | | | |
| EFEMP1 | HSG00213609 | 10025903996 | EGF-containing | DRAD; FBLN3; | −0.640 | 0.020 | 0.587 | 0.002 |
| | | | fibulin-like | FBNL; fibulin-3; | | | | |
| | | | extracellular matrix | FLJ35535; | | | | |
| | | | protein 1 | MGC111353; | | | | |
| | | | | MLVT; MTLV; | | | | |
| | | | | S1-5; DHRD | | | | |
| C1orf2 | HSG00208196 | 10023808153 | chromosome 1 open | cote; cote_1; | −0.646 | 0.019 | 0.521 | 0.007 |
| | | | reading frame 2 | COTE1; 1 | | | | |
| SUSD4 | HSG00202974 | 10023808649 | sushi domain | FLJ10052; | −0.651 | 0.018 | 0.600 | 0.001 |
| | | | containing 4 | PRO222; RP11- | | | | |
| | | | | 239E10.4; | | | | |
| | | | | (FLJ10052) | | | | |
| CD300A | HSG00301937 | 10025906933 | CD300a molecule | CMRF-35H; | −0.654 | 0.017 | 0.450 | 0.021 |
| | | | | CMRF35H; | | | | |
| | | | | CMRF35H9; | | | | |
| | | | | IGSF12; IRC1; | | | | |
| | | | | IRC2; IRp60; | | | | |
| | | | | CMRF-35-H9 | | | | |
| MAP4K4 | HSG00214314 | 10025909093 | mitogen-activated | FLH21957; | −0.661 | 0.015 | 0.431 | 0.027 |
| | | | protein kinase | FLJ10410; | | | | |
| | | | kinase kinase | FLJ20373; | | | | |
| | | | kinase 4 | FLJ90111; HGK; | | | | |
| | | | | KIAA0687; NIK; | | | | |
| | | | | ZC1; (NIK) | | | | |
| C20orf6 | HSG00311565 | 10025919052 | | | −0.667 | 0.014 | 0.538 | 0.005 |
| C12orf30 | HSG00276920 | 10023814776 | chromosome 12 | FLJ13089; | −0.668 | 0.014 | 0.418 | 0.033 |
| | | | open reading frame | DKFZp667K2112 | | | | |
| | | | 30 | | | | | |
| NOTCH3 | HSG00305545 | 10025905290 | Notch homolog 3 | CASIL; | −0.679 | 0.012 | 0.762 | 0.000010 |
| | | | (Drosophila) | CADASIL | | | | |
| NaN | HSG00277925 | 10023843307 | | | −0.687 | 0.011 | 0.567 | 0.003 |
| NaN | HSG00400935 | 10023834463 | | | −0.693 | 0.010 | 0.546 | 0.004 |
| NaN | HSG00295946 | 10025921852 | | | −0.694 | 0.010 | 0.442 | 0.024 |
| RUVBL1 | HSG00221703 | 10025904348 | RuvB-like 1 (E. coli) | NMP238; RVB1; | −0.696 | 0.009 | 0.570 | 0.003 |
| | | | | TAP54alpha; | | | | |
| | | | | TIP49; TIP49A; | | | | |
| | | | | ECP54 | | | | |
| NaN | HSG00214987 | 10025902175 | | | −0.703 | 0.009 | 0.638 | 0.001 |
| DTX1 | HSG00277754 | 10023826610 | deltex homolog 1 | hDx-1 | −0.708 | 0.008 | 0.746 | 0.000019 |
| | | | (Drosophila) | | | | | |
| FAM121B | HSG00255115 | 10023848086 | | | −0.714 | 0.007 | 0.609 | 0.001 |
| C6orf79 | HSG00242495 | 10023837483 | | | −0.740 | 0.005 | 0.464 | 0.017 |
| NaN | HSG00290947 | 10025924862 | | | −0.744 | 0.004 | 0.696 | 0.000110 |
| NaN | HSG00375622 | 10025930900 | | | −0.763 | 0.003 | 0.708 | 0.000076 |
| TASP1 | HSG00311850 | 10023827451 | taspase, threonine | dJ585I14.2; | −0.774 | 0.002 | 0.637 | 0.001 |
| | | | aspartase, 1 | FLJ20212; | | | | |
| | | | | MGC39159; | | | | |
| | | | | C20orf13 | | | | |
| C20orf6 | HSG00311565 | 10025908942 | | | −0.781 | 0.002 | 0.463 | 0.017 |
| NaN | HSG00217876 | 10025915073 | | | −0.784 | 0.002 | 0.435 | 0.026 |
| CHCHD6 | HSG00219957 | 10023836906 | coiled-coil-helix- | MGC13016 | −0.815 | 0.001 | 0.651 | 0.000409 |
| | | | coiled-coil-helix | | | | | |
| | | | domain containing 6 | | | | | |
| NaN | HSG00300366 | 10025927694 | | | −0.825 | 0.001 | 0.596 | 0.002 |

TABLE 7
Genes which are positively correlated with GSI-sensitivity of cells (expression was lower in
sensitive cells) and negatively correlated with GSI treatment (expression was increased by GSI
treatment)

| | | | | | | | GI50 | |
|---|---|---|---|---|---|---|---|---|
| | | | | | GI50 | | Correla- | |
| Gene | Gene | | | Gene/Alternate | Correlation | p-value | tion | p-value |
| Symbol | Model | Transcript | Gene/Name | Gene Symbol | Predose | Predose | Postdose | Postdose |
| DENND3 | HSG00355277 | 10023821538 | DENN/MADD | Hs.18166; | 0.844 | 0.000 | −0.576 | 0.002 |
| | | | domain | KIAA0870; | | | | |
| | | | containing 3 | DKFZP586I2121 | | | | |
| NaN | HSG00417626 | 10023816238 | | | 0.808 | 0.001 | −0.471 | 0.015 |
| NaN | HSG00278444 | 10025935291 | | | 0.804 | 0.001 | −0.404 | 0.039 |
| SFXN5 | HSG00211827 | 10025905887 | sideroflexin 5 | MGC120413; | 0.791 | 0.002 | −0.752 | 0.000 |
| | | | | MGC120415; | | | | |
| | | | | BBG-TCC | | | | |
| SPOCK2 | HSG00269114 | 10023824933 | sparc/osteonectin, | testican-2; | 0.781 | 0.002 | −0.475 | 0.014 |
| | | | cwcv and | KIAA0275 | | | | |
| | | | kazal-like | | | | | |
| | | | domains | | | | | |
| | | | proteoglycan | | | | | |
| | | | (testican) 2 | | | | | |
| SULT1A3 | HSG00296408 | 10023818392 | sulfotransferase | HAST; HAST3; | 0.781 | 0.002 | −0.506 | 0.009 |
| | | | family, cytosolic, | LOC648394; M- | | | | |
| | | | 1A, phenol- | PST; | | | | |
| | | | preferring, | MGC117469; | | | | |
| | | | member 3 | ST1A5; STM; | | | | |
| | | | | STM.; SULT1A4; | | | | |
| | | | | TL-PST; | | | | |
| | | | | (SULT1A4) | | | | |
| MYH9 | HSG00314072 | 10025912958 | myosin, heavy | DFNA17; | 0.763 | 0.003 | −0.407 | 0.038 |
| | | | chain 9, non- | EPSTS; FTNS; | | | | |
| | | | muscle | Hs.146550; | | | | |
| | | | | MGC104539; | | | | |
| | | | | MHA; NMHC; | | | | |
| | | | | NMHC-II-A; | | | | |
| | | | | NMMHC-IIa; | | | | |
| | | | | NMMHCA; | | | | |
| | | | | (myosin) | | | | |
| RASSF1 | HSG00221695 | 10025910157 | Ras association | NORE2A; | 0.753 | 0.004 | −0.430 | 0.028 |
| | | | (RalGDS/AF-6) | RASSF1A; | | | | |
| | | | domain family 1 | RASSF1B; | | | | |
| | | | | RASSF1C; | | | | |
| | | | | RDA32; | | | | |
| | | | | REH3P21; | | | | |
| | | | | 123F2 | | | | |
| HPS3 | HSG00225853 | 10023850169 | Hermansky- | FLJ22704; | 0.748 | 0.004 | −0.668 | 0.000 |
| | | | Pudlak | SUTAL; | | | | |
| | | | syndrome 3 | DKFZp686F0413 | | | | |
| TRIM39 | HSG00458889 | 10025910852 | tripartite motif- | MGC32984; | 0.745 | 0.004 | −0.421 | 0.032 |
| | | | containing 39 | RNF23; TFP; | | | | |
| | | | | TRIM39B; (TFP) | | | | |
| ERCC5 | HSG00284660 | 10025905676 | excision repair | ERCM2; UVDR; | 0.728 | 0.006 | −0.545 | 0.004 |
| | | | cross- | XPG; XPGC; | | | | |
| | | | complementing | COFS3 | | | | |
| | | | rodent repair | | | | | |
| | | | deficiency, | | | | | |
| | | | complementation | | | | | |
| | | | group 5 | | | | | |
| | | | (xeroderma | | | | | |
| | | | pigmentosum, | | | | | |
| | | | complementation | | | | | |
| | | | group G | | | | | |
| | | | (Cockayne | | | | | |
| | | | syndrome)) | | | | | |
| SULT1A3 | HSG00296408 | 10025909341 | sulfotransferase | HAST; HAST3; | 0.727 | 0.006 | −0.512 | 0.008 |
| | | | family, cytosolic, | LOC648394; M- | | | | |
| | | | 1A, phenol- | PST; | | | | |
| | | | preferring, | MGC117469; | | | | |
| | | | member 3 | ST1A5; STM; | | | | |
| | | | | STM.; SULT1A4; | | | | |
| | | | | TL-PST; | | | | |
| | | | | (SULT1A4) | | | | |
| NaN | HSG00290423 | 10025925020 | | | 0.725 | 0.006 | −0.473 | 0.015 |
| STK11IP | HSG00212413 | 10025902398 | serine/threonine | KIAA1898; LIP1; | 0.718 | 0.007 | −0.520 | 0.007 |
| | | | kinase 11 | LKB1IP; | | | | |
| | | | interacting | STK11IP1; | | | | |
| | | | protein | (LIP1) | | | | |
| ANKRD15 | HSG00260920 | 10025909256 | ankyrin repeat | DKFZp451G231; | 0.713 | 0.007 | −0.530 | 0.006 |
| | | | domain 15 | Hs.77546; | | | | |
| | | | | KANK; | | | | |
| | | | | KIAA0172; | | | | |
| | | | | MGC43128; | | | | |
| | | | | (KIAA0172) | | | | |
| ZNF683 | HSG00204422 | 10025912827 | zinc finger | RP11-569G9.6; | 0.710 | 0.008 | −0.442 | 0.024 |
| | | | protein 683 | MGC33414 | | | | |
| C10orf118 | HSG00268767 | 10023820116 | chromosome 10 | FLJ35301; | 0.709 | 0.008 | −0.640 | 0.001 |
| | | | open reading | Hs.159066; | | | | |
| | | | frame 118 | MGC118918; | | | | |
| | | | | MGC129699; | | | | |
| | | | | FLJ10188 | | | | |
| CENTG1 | HSG00277315 | 10023827980 | centaurin, | FLJ16430; | 0.707 | 0.008 | −0.656 | 0.000 |
| | | | gamma 1 | GGAP2; | | | | |
| | | | | KIAA0167; PIKE; | | | | |
| | | | | AGAP2 | | | | |
| SH3BP1 | HSG00314321 | 10025911251 | SH3-domain | dJ37E16.2; | 0.707 | 0.008 | −0.514 | 0.008 |
| | | | binding protein 1 | FLJ21318; | | | | |
| | | | | dJ37E16 | | | | |
| NADSYN1 | HSG00272334 | 10025913128 | NAD synthetase 1 | FLJ36703; | 0.703 | 0.009 | −0.495 | 0.010 |
| | | | | FLJ40627; | | | | |
| | | | | FLJ10631 | | | | |
| AOAH | HSG00250049 | 10025909535 | acyloxyacyl | | 0.698 | 0.009 | −0.419 | 0.032 |
| | | | hydrolase | | | | | |
| | | | (neutrophil) | | | | | |
| ANKZF1 | HSG00214093 | 10023816256 | ankyrin repeat | FLJ13144; | 0.698 | 0.009 | −0.463 | 0.017 |
| | | | and zinc finger | ZNF744; | | | | |
| | | | domain | FLJ10415 | | | | |
| | | | containing 1 | | | | | |
| SULT1A1 | HSG00296644 | 10023804824 | sulfotransferase | (PST); (STP); | 0.697 | 0.009 | −0.464 | 0.017 |
| | | | family, cytosolic, | HAST1/HAST2; | | | | |
| | | | 1A, phenol- | HAST1_HAST2; | | | | |
| | | | preferring, | MGC131921; | | | | |
| | | | member 1 | MGC5163; P- | | | | |
| | | | | PST; P-PST1; | | | | |
| | | | | PST; ST1A3; | | | | |
| | | | | STP; STP1; | | | | |
| | | | | TSPST1; (P- | | | | |
| | | | | PST) | | | | |
| STARD9 | HSG00289593 | 10023816073 | StAR-related | FLJ16106; | 0.694 | 0.010 | −0.595 | 0.002 |
| | | | lipid transfer | FLJ21936; | | | | |
| | | | (START) domain | KIAA1300; | | | | |
| | | | containing 9 | DKFZp781J069 | | | | |
| SULT1A2 | HSG00296838 | 10023807348 | sulfotransferase | (PST); HAST4; | 0.694 | 0.010 | −0.450 | 0.021 |
| | | | family, cytosolic, | MGC142287; | | | | |
| | | | 1A, phenol- | MGC142289; P- | | | | |
| | | | preferring, | PST; ST1A2; | | | | |
| | | | member 2 | STP2; TSPST2; | | | | |
| | | | | (P-PST) | | | | |
| U2AF1L2 | HSG00254882 | 10025904719 | zinc finger | MGC142040; | 0.689 | 0.010 | −0.466 | 0.016 |
| | | | (CCCH type), | U2AF1-RS2; | | | | |
| | | | RNA-binding | U2AF1L2; | | | | |
| | | | motif and | U2AF1RS2; | | | | |
| | | | serine/arginine | URP; | | | | |
| | | | rich 2 | MGC142014 | | | | |
| LCP2 | HSG00232472 | 10023809856 | lymphocyte | SLP76; SLP-76 | 0.688 | 0.011 | −0.526 | 0.006 |
| | | | cytosolic protein | | | | | |
| | | | 2 (SH2 domain | | | | | |
| | | | containing | | | | | |
| | | | leukocyte protein | | | | | |
| | | | of 76 kDa) | | | | | |
| KIAA1914 | HSG00269212 | 10025903072 | | | 0.688 | 0.011 | −0.702 | 0.000 |
| SULT1A3 | HSG00296408 | 10033668549 | sulfotransferase | HAST; HAST3; | 0.687 | 0.011 | −0.493 | 0.011 |
| | | | family, cytosolic, | LOC648394; M- | | | | |
| | | | 1A, phenol- | PST; | | | | |
| | | | preferring, | MGC117469; | | | | |
| | | | member 3 | ST1A5; STM; | | | | |
| | | | | STM.; SULT1A4; | | | | |
| | | | | TL-PST; | | | | |
| | | | | (SULT1A4) | | | | |
| RBM4B | HSG00273410 | 10025909252 | RNA binding | RBM30; RBM4L; | 0.687 | 0.011 | −0.652 | 0.000 |
| | | | motif protein 4B | ZCCHC15; | | | | |
| | | | | ZCRB3B; | | | | |
| | | | | MGC10871 | | | | |
| ITSN2 | HSG00214345 | 10025902436 | intersectin 2 | KIAA1256; | 0.686 | 0.011 | −0.755 | 0.000 |
| | | | | SH3D1B; | | | | |
| | | | | SH3P18; SWA; | | | | |
| | | | | SWAP; (SWAP) | | | | |
| NaN | HSG00234205 | 10023834496 | | | 0.685 | 0.011 | −0.575 | 0.002 |
| CCDC88 | HSG00272791 | 10023830292 | | | 0.679 | 0.012 | −0.545 | 0.004 |
| IL16 | HSG00289749 | 10025906384 | interleukin 16 | FLJ42735; | 0.678 | 0.012 | −0.679 | 0.000 |
| | | | (lymphocyte | FLJ44234; | | | | |
| | | | chemoattractant | HsT19289; IL- | | | | |
| | | | factor) | 16; LCF; prIL-16; | | | | |
| | | | | FLJ16806 | | | | |
| NaN | HSG00494309 | 10025925290 | | | 0.677 | 0.012 | −0.430 | 0.028 |
| LRP10 | HSG00287753 | 10025905083 | low density | DKFZP564C1940; | 0.672 | 0.013 | −0.603 | 0.001 |
| | | | lipoprotein | LRP9; | | | | |
| | | | receptor-related | MGC142274; | | | | |
| | | | protein 10 | MGC142276; | | | | |
| | | | | MGC8675; | | | | |
| | | | | MST087; | | | | |
| | | | | MSTP087; | | | | |
| | | | | (LRP9) | | | | |
| SNX14 | HSG00243607 | 10025910751 | sorting nexin 14 | RGS-PX2; | 0.672 | 0.013 | −0.430 | 0.028 |
| | | | | RP11-321N4.2; | | | | |
| | | | | MGC13217 | | | | |
| SSH3 | HSG00271818 | 10023812453 | slingshot | FLJ20515; | 0.672 | 0.013 | −0.407 | 0.038 |
| | | | homolog 3 | Hs.29173; SSH- | | | | |
| | | | (Drosophila) | 3; FLJ10928 | | | | |
| NaN | HSG00448506 | 10025930023 | | | 0.671 | 0.013 | −0.522 | 0.007 |
| AACS | HSG00277772 | 10025906010 | acetoacetyl-CoA | FLJ41251; SUR- | 0.664 | 0.015 | −0.518 | 0.007 |
| | | | synthetase | 5; FLJ12389 | | | | |
| CPT1A | HSG00272329 | 10023809543 | carnitine | CPT1; CPT1-L; | 0.664 | 0.015 | −0.405 | 0.039 |
| | | | palmitoyltransferase | L-CPT1; (CPT1) | | | | |
| | | | 1A (liver) | | | | | |
| ABLIM1 | HSG00268756 | 10023818148 | actin binding LIM | ABLIM; | 0.664 | 0.015 | −0.444 | 0.023 |
| | | | protein 1 | DKFZp781D0148; | | | | |
| | | | | FLJ14564; | | | | |
| | | | | KIAA0059; | | | | |
| | | | | LIMAB1; | | | | |
| | | | | LIMATIN; | | | | |
| | | | | MGC1224; | | | | |
| | | | | (FLJ14564) | | | | |
| ATP2C1 | HSG00226491 | 10025911572 | ATPase, Ca++ | BCPM; HHD; | 0.662 | 0.015 | −0.509 | 0.008 |
| | | | transporting, | hSPCA1; | | | | |
| | | | type 2C, | KIAA1347; | | | | |
| | | | member 1 | PMR1; PMR1L; | | | | |
| | | | | SPCA1; | | | | |
| | | | | ATP2C1A | | | | |
| TRIP4 | HSG00290999 | 10025908500 | thyroid hormone | LOC51694; | 0.657 | 0.016 | −0.724 | 0.000 |
| | | | receptor | HsT17391 | | | | |
| | | | interactor 4 | | | | | |
| FAM107B | HSG00269642 | 10023849056 | family with | FLJ45505; | 0.653 | 0.017 | −0.709 | 0.000 |
| | | | sequence | MGC11034; | | | | |
| | | | similarity 107, | MGC90261; | | | | |
| | | | member B | C10orf45 | | | | |
| RAD9B | HSG00278291 | 10025908502 | RAD9 homolog | Hs.97794; | 0.653 | 0.017 | −0.400 | 0.041 |
| | | | B (S. cerevisiae) | MGC75426; | | | | |
| | | | | FLJ40346 | | | | |
| RGL2 | HSG00459537 | 10025906964 | ral guanine | HKE1.5; KE1.5; | 0.653 | 0.017 | −0.557 | 0.003 |
| | | | nucleotide | RAB2L; Rg1; Rlf; | | | | |
| | | | dissociation | (RAB2) | | | | |
| | | | stimulator-like 2 | | | | | |
| NaN | HSG00507879 | 10025922834 | | | 0.641 | 0.020 | −0.477 | 0.014 |
| GGA1 | HSG00314283 | 10023821470 | golgi associated, | DKFZP434A033 | 0.641 | 0.020 | −0.438 | 0.025 |
| | | | gamma adaptin | | | | | |
| | | | ear containing, | | | | | |
| | | | ARF binding | | | | | |
| | | | protein 1 | | | | | |
| CASC5 | HSG00289772 | 10025909958 | cancer | D40; Hs.283099; | 0.640 | 0.020 | −0.468 | 0.016 |
| | | | susceptibility | KIAA1570; | | | | |
| | | | candidate 5 | AF15Q14 | | | | |
| ABLIM1 | HSG00268756 | 10023820886 | actin binding LIM | ABLIM; | 0.640 | 0.020 | −0.428 | 0.028 |
| | | | protein 1 | DKFZp781D0148; | | | | |
| | | | | FLJ14564; | | | | |
| | | | | KIAA0059; | | | | |
| | | | | LIMAB1; | | | | |
| | | | | LIMATIN; | | | | |
| | | | | MGC1224; | | | | |
| | | | | (FLJ14564) | | | | |
| ABCA2 | HSG00260652 | 10033668986 | ATP-binding | KIAA1062; | 0.638 | 0.021 | −0.462 | 0.018 |
| | | | cassette, sub- | MGC129761; | | | | |
| | | | family A (ABC1), | ABC2 | | | | |
| | | | member 2 | | | | | |
| PTPRC | HSG00207284 | 10023811643 | protein tyrosine | B220; CD45; | 0.638 | 0.021 | −0.425 | 0.030 |
| | | | phosphatase, | GP180; LCA; | | | | |
| | | | receptor type, C | LY5; T200; | | | | |
| | | | | (LCA) | | | | |
| SRGAP3 | HSG00224812 | 10025913189 | SLIT-ROBO Rho | (srGAP3); | 0.638 | 0.021 | −0.509 | 0.008 |
| | | | GTPase | ARHGAP14; | | | | |
| | | | activating protein 3 | KIAA0411; | | | | |
| | | | | MEGAP; | | | | |
| | | | | SRGAP2; WRP; | | | | |
| | | | | (SRGAP2) | | | | |
| RBM38 | HSG00311824 | 10023841432 | RNA binding | HSRNASEB; | 0.637 | 0.021 | −0.550 | 0.004 |
| | | | motif protein 38 | RNPC1; SEB4B; | | | | |
| | | | | SEB4D; | | | | |
| | | | | dJ800J21.2 | | | | |
| MANBA | HSG00228144 | 10025907439 | mannosidase, | MANB1 | 0.637 | 0.021 | −0.590 | 0.002 |
| | | | beta A, | | | | | |
| | | | lysosomal | | | | | |
| ABCA2 | HSG00260652 | 10025906623 | ATP-binding | KIAA1062; | 0.636 | 0.021 | −0.411 | 0.036 |
| | | | cassette, sub- | MGC129761; | | | | |
| | | | family A (ABC1), | ABC2 | | | | |
| | | | member 2 | | | | | |
| NaN | HSG00456980 | 10025913782 | | | 0.633 | 0.022 | −0.576 | 0.002 |
| CCDC15 | HSG00272036 | 10023833567 | coiled-coil | FLJ13215 | 0.631 | 0.023 | −0.490 | 0.011 |
| | | | domain | | | | | |
| | | | containing 15 | | | | | |
| MYADML | HSG00215659 | 10023838164 | myeloid-associated differentiation | | 0.628 | 0.023 | −0.432 | 0.027 |
| | | | marker-like | | | | | |
| SLC16A10 | HSG00244608 | 10033668841 | solute carrier | PRO0813; | 0.628 | 0.023 | −0.678 | 0.000 |
| | | | family 16, | TAT1; (TAT1) | | | | |
| | | | member 10 | | | | | |
| | | | (aromatic amino | | | | | |
| | | | acid transporter) | | | | | |
| NaN | HSG00412423 | 10025920332 | | | 0.627 | 0.024 | −0.531 | 0.006 |
| ANXA11 | HSG00268040 | 10025902722 | annexin A11 | CAP50; ANX11 | 0.626 | 0.024 | −0.413 | 0.035 |
| NaN | HSG00262113 | 10025913979 | | | 0.625 | 0.024 | −0.457 | 0.019 |
| NaN | HSG00207626 | 10025926957 | | | 0.621 | 0.025 | −0.440 | 0.024 |
| LIME1 | HSG00312359 | 10023851065 | Lck interacting | FLJ20406; LIME; | 0.620 | 0.026 | −0.404 | 0.039 |
| | | | transmembrane | LP8067; RP4- | | | | |
| | | | adaptor 1 | 583P15.5; | | | | |
| | | | | dJ583P15.4 | | | | |
| NaN | HSG00201721 | 10025915400 | | | 0.619 | 0.026 | −0.470 | 0.016 |
| CCNE1 | HSG00307982 | 10025906388 | cyclin E1 | CCNE | 0.619 | 0.026 | −0.434 | 0.026 |
| CDK9 | HSG00262688 | 10025904660 | cyclin-dependent | CDC2L4; | 0.618 | 0.026 | −0.571 | 0.003 |
| | | | kinase 9 (CDC2- | PITALRE; TAK; | | | | |
| | | | related kinase) | C-2k | | | | |
| ARHGAP9 | HSG00278251 | 10025911993 | Rho GTPase | 10C; FLJ16525; | 0.616 | 0.027 | −0.636 | 0.001 |
| | | | activating protein 9 | MGC1295; | | | | |
| | | | | RGL1; (RGL1) | | | | |
| NaN | HSG00223816 | 10025914956 | | | 0.616 | 0.027 | −0.584 | 0.002 |
| NaN | HSG00234550 | 10025934978 | | | 0.612 | 0.028 | −0.437 | 0.025 |
| SPECC1 | HSG00302882 | 10025911310 | sperm antigen | FLJ36955; | 0.610 | 0.029 | −0.527 | 0.006 |
| | | | with calponin | HCMOGT-1; | | | | |
| | | | homology and | NSP; (NSP) | | | | |
| | | | coiled-coil | | | | | |
| | | | domains 1 | | | | | |
| HDAC5 | HSG00301641 | 10025908560 | histone | HD5; KIAA0600; | 0.607 | 0.029 | −0.590 | 0.002 |
| | | | deacetylase 5 | NY-CO-9; | | | | |
| | | | | FLJ90614 | | | | |
| ADAM8 | HSG00267564 | 10023818002 | ADAM | MGC134985; | 0.607 | 0.029 | −0.460 | 0.018 |
| | | | metallopeptidase | MS2; CD156 | | | | |
| | | | domain 8 | | | | | |
| TNFSF13B | HSG00285025 | 10033668475 | tumor necrosis | BLYS; CD257; | 0.606 | 0.030 | −0.501 | 0.010 |
| | | | factor (ligand) | delta; TALL-1; | | | | |
| | | | superfamily, | TALL1; THANK; | | | | |
| | | | member 13b | TNFSF20; | | | | |
| | | | | ZTNF4; BAFF | | | | |
| TFF3 | HSG00315974 | 10023827487 | trefoil factor 3 | HITF; hP1.B; | 0.604 | 0.030 | −0.614 | 0.001 |
| | | | (intestinal) | Hs.82961; ITF; | | | | |
| | | | | TFI; (TFI) | | | | |
| NaN | HSG00272097 | 10023835412 | | | 0.603 | 0.031 | −0.594 | 0.002 |
| NaN | HSG00245802 | 10025916827 | | | 0.601 | 0.031 | −0.484 | 0.012 |
| SEC11L1 | HSG00291625 | 10025912981 | SEC11 homolog | SEC11L1; | 0.596 | 0.033 | −0.491 | 0.011 |
| | | | A (S. cerevisiae) | sid2895; SPC18; | | | | |
| | | | | SPCS4A; | | | | |
| | | | | 1810012E07Rik | | | | |
| TRERF1 | HSG00243465 | 10025912467 | transcriptional | dJ139D8.5; | 0.595 | 0.034 | −0.438 | 0.025 |
| | | | regulating factor 1 | HSA277276; | | | | |
| | | | | RAPA; RP1- | | | | |
| | | | | 139D8.5; TReP- | | | | |
| | | | | 132; BCAR2 | | | | |
| NaN | HSG00267998 | 10025933169 | | | 0.594 | 0.034 | −0.562 | 0.003 |
| DNAJC1 | HSG00268617 | 10023849098 | DnaJ (Hsp40) | ERdj1; HTJ1; | 0.593 | 0.034 | −0.456 | 0.019 |
| | | | homolog, | MGC131954; | | | | |
| | | | subfamily C, | DNAJL1 | | | | |
| | | | member 1 | | | | | |
| GPR44 | HSG00271773 | 10023822471 | G protein- | CRTH2; CD294 | 0.593 | 0.034 | −0.548 | 0.004 |
| | | | coupled receptor | | | | | |
| | | | 44 | | | | | |
| ATF7IP | HSG00277034 | 10025907474 | activating | FLJ10139; | 0.593 | 0.034 | −0.439 | 0.025 |
| | | | transcription | FLJ10688; | | | | |
| | | | factor 7 | MCAF; p621; | | | | |
| | | | interacting | (MCAF) | | | | |
| | | | protein | | | | | |
| CCNB2 | HSG00291582 | 10023817096 | cyclin B2 | HsT17299 | 0.593 | 0.034 | −0.439 | 0.024 |
| PPM1B | HSG00213623 | 10025912990 | protein | MGC21657; | 0.592 | 0.035 | −0.660 | 0.000 |
| | | | phosphatase 1B | PP2C-beta-X; | | | | |
| | | | (formerly 2C), | PP2CB; | | | | |
| | | | magnesium- | PP2CBETA; | | | | |
| | | | dependent, beta | PP2CBETAX; | | | | |
| | | | isoform | PPC2BETAX; | | | | |
| | | | | (PP2CB) | | | | |
| RASSF1 | HSG00221695 | 10023819349 | Ras association | NORE2A; | 0.591 | 0.035 | −0.431 | 0.027 |
| | | | (RalGDS/AF-6) | RASSF1A; | | | | |
| | | | domain family 1 | RASSF1B; | | | | |
| | | | | RASSF1C; | | | | |
| | | | | RDA32; | | | | |
| | | | | REH3P21; | | | | |
| | | | | 123F2 | | | | |
| NaN | HSG00357512 | 10025920975 | | | 0.590 | 0.035 | −0.458 | 0.019 |
| NaN | HSG00296979 | 10025928968 | | | 0.589 | 0.036 | −0.520 | 0.007 |
| NaN | HSG00229845 | 10023821239 | | | 0.589 | 0.036 | −0.404 | 0.039 |
| TNFSF13B | HSG00285025 | 10023821604 | tumor necrosis | BLYS; CD257; | 0.589 | 0.036 | −0.598 | 0.001 |
| | | | factor (ligand) | delta; TALL-1; | | | | |
| | | | superfamily, | TALL1; THANK; | | | | |
| | | | member 13b | TNFSF20; | | | | |
| | | | | ZTNF4; BAFF | | | | |
| HEMGN | HSG00262315 | 10025906806 | hemogen | EDAG-1; | 0.587 | 0.037 | −0.787 | 0.000 |
| | | | | Hs.176626; | | | | |
| | | | | EDAG | | | | |
| NaN | HSG00251346 | 10025904602 | | | 0.587 | 0.037 | −0.558 | 0.003 |
| TRIP11 | HSG00287191 | 10023823142 | thyroid hormone | GMAP-210; | 0.585 | 0.037 | −0.596 | 0.002 |
| | | | receptor | TRIP230; | | | | |
| | | | interactor 11 | CEV14 | | | | |
| STK10 | HSG00237200 | 10025908110 | serine/threonine | PRO2729; LOK | 0.585 | 0.037 | −0.516 | 0.007 |
| | | | kinase 10 | | | | | |
| NaN | HSG00272717 | 10025909547 | | | 0.585 | 0.038 | −0.524 | 0.006 |
| FBXL12 | HSG00308262 | 10023810278 | F-box and | FLJ20188; Fbl12 | 0.584 | 0.038 | −0.589 | 0.002 |
| | | | leucine-rich | | | | | |
| | | | repeat protein 12 | | | | | |
| NaN | | 10025933345 | | | 0.583 | 0.038 | −0.490 | 0.011 |
| GSN | HSG00261914 | 10023823864 | gelsolin | DKFZp313L0718 | 0.583 | 0.038 | −0.779 | 0.000 |
| | | | (amyloidosis, | | | | | |
| | | | Finnish type) | | | | | |
| CAPN3 | HSG00291349 | 10025905287 | calpain 3, (p94) | CANPL3; | 0.582 | 0.039 | −0.623 | 0.001 |
| | | | | LGMD2; | | | | |
| | | | | LGMD2A; | | | | |
| | | | | MGC10767; | | | | |
| | | | | MGC11121; | | | | |
| | | | | MGC14344; | | | | |
| | | | | MGC4403; nCL- | | | | |
| | | | | 1; p94; CANP3 | | | | |
| BIN2 | HSG00282359 | 10023825264 | bridging | LOC51722; | 0.581 | 0.039 | −0.415 | 0.034 |
| | | | integrator 2 | BRAP-1 | | | | |
| LRCH4 | HSG00248461 | 10025902716 | leucine-rich | FLJ40101; | 0.581 | 0.039 | −0.469 | 0.016 |
| | | | repeats and | FLJ46315; LRN; | | | | |
| | | | calponin | LRRN1; LRRN4; | | | | |
| | | | homology (CH) | PP14183; | | | | |
| | | | domain | SAP25; (LRRN1) | | | | |
| | | | containing 4 | | | | | |
| NaN | HSG00262396 | 10025930047 | | | 0.580 | 0.039 | −0.612 | 0.001 |
| TMEM161B | HSG00238348 | 10025903356 | transmembrane | FLB3342; | 0.579 | 0.040 | −0.433 | 0.027 |
| | | | protein 161B | MGC33214; | | | | |
| | | | | PRO1313; | | | | |
| | | | | (FLB3342) | | | | |
| NaN | HSG00277032 | 10025915718 | | | 0.578 | 0.040 | −0.507 | 0.009 |
| MADD | HSG00272747 | 10023822286 | MAP-kinase | DENN-SV; IG20; | 0.578 | 0.040 | −0.636 | 0.001 |
| | | | activating death | IG20-PA; IG20- | | | | |
| | | | domain | SV1; IG20-SV2; | | | | |
| | | | | IG20-SV3; IG20- | | | | |
| | | | | SV4; KIAA0358; | | | | |
| | | | | RAB3GEP; | | | | |
| | | | | DENN | | | | |
| PLEKHK1 | HSG00268758 | 10025903212 | pleckstrin | DKFZp686J10120; | 0.577 | 0.041 | −0.586 | 0.002 |
| | | | homology | FLJ39352; | | | | |
| | | | domain | LOC219790; | | | | |
| | | | containing, | RTKN2; | | | | |
| | | | family K member 1 | bA531F24.1 | | | | |
| KIAA0141 | HSG00237866 | 10023827598 | KIAA0141 | | 0.573 | 0.042 | −0.561 | 0.003 |
| C20orf195 | HSG00312379 | 10023831581 | chromosome 20 | MGC5356; | 0.572 | 0.043 | −0.478 | 0.014 |
| | | | open reading | Hs.197755 | | | | |
| | | | frame 195 | | | | | |
| BACH2 | HSG00242604 | 10023827472 | BTB and CNC homology 1, basic | | 0.570 | 0.043 | −0.419 | 0.032 |
| | | | leucine zipper transcription factor 2 | | | | | |
| CD52 | HSG00208781 | 10025905651 | CD52 molecule | CDW52 | 0.569 | 0.044 | −0.574 | 0.002 |
| NaN | HSG00233699 | 10023845979 | | | 0.568 | 0.044 | −0.525 | 0.006 |
| NaN | HSG00249949 | 10023836558 | | | 0.568 | 0.044 | −0.504 | 0.009 |
| NaN | HSG00510386 | 10025930194 | | | 0.567 | 0.045 | −0.446 | 0.022 |
| C1orf24 | HSG00201300 | 10025906960 | | | 0.566 | 0.045 | −0.446 | 0.022 |
| SMARCA2 | HSG00260706 | 10023820977 | SWI/SNF | BAF190; BRM; | 0.566 | 0.045 | −0.406 | 0.039 |
| | | | related, matrix | FLJ36757; | | | | |
| | | | associated, actin | hBRM; hSNF2a; | | | | |
| | | | dependent | MGC74511; | | | | |
| | | | regulator of | SNF2; SNF2L2; | | | | |
| | | | chromatin, | SNF2LA; Sth1p; | | | | |
| | | | subfamily a, | SWI2; (BAF190) | | | | |
| | | | member 2 | | | | | |
| TBL1XR1 | HSG00226005 | 10025908867 | transducin | DC42; | 0.566 | 0.045 | −0.537 | 0.005 |
| | | | (beta)-like 1X- | FLJ12894; IRA1; | | | | |
| | | | linked receptor 1 | TBLR1; C21 | | | | |
| CASP9 | HSG00203112 | 10025909668 | caspase 9, | APAF3; Cas-9; | 0.565 | 0.046 | −0.483 | 0.013 |
| | | | apoptosis- | caspase-9; | | | | |
| | | | related cysteine | CASPASE-9c; | | | | |
| | | | peptidase | ICE-LAP6; | | | | |
| | | | | MCH6; APAF-3 | | | | |
| TCF20 | HSG00314071 | 10025911643 | transcription | KIAA0292; | 0.565 | 0.046 | −0.601 | 0.001 |
| | | | factor 20 (AR1) | SPBP; AR1 | | | | |
| RHOH | HSG00227058 | 10023812089 | ras homolog | TTF; ARHH | 0.563 | 0.046 | −0.605 | 0.001 |
| | | | gene family, | | | | | |
| | | | member H | | | | | |
| ZC3H12A | HSG00209124 | 10023840535 | zinc finger | FLJ23231; | 0.563 | 0.047 | −0.408 | 0.037 |
| | | | CCCH-type | MCPIP; RP3- | | | | |
| | | | containing 12A | 423B22.1; | | | | |
| | | | | dJ423B22.1 | | | | |
| NaN | HSG00427384 | 10025927558 | | | 0.562 | 0.047 | −0.576 | 0.002 |
| BRPF1 | HSG00225891 | 10025912016 | bromodomain | PEREGRIN; | 0.562 | 0.047 | −0.444 | 0.023 |
| | | | and PHD finger | BR140 | | | | |
| | | | containing, 1 | | | | | |
| NaN | HSG00296311 | 10025903387 | | | 0.562 | 0.047 | −0.611 | 0.001 |
| GMEB1 | HSG00203614 | 10025911137 | glucocorticoid | p96; P96PIF; | 0.559 | 0.048 | −0.481 | 0.013 |
| | | | modulatory | PIF96; GMEB- | | | | |
| | | | element binding | 1_prime | | | | |
| | | | protein 1 | | | | | |
| CCPG1 | HSG00290603 | 10025903075 | cell cycle | KIAA1254; | 0.559 | 0.049 | −0.420 | 0.032 |
| | | | progression 1 | CPR8 | | | | |
| PNRC1 | HSG00242056 | 10023816478 | proline-rich | B4-2; Hs.75969; | 0.558 | 0.049 | −0.598 | 0.001 |
| | | | nuclear receptor | PNAS-145; | | | | |
| | | | coactivator 1 | PROL2; PRR2; | | | | |
| | | | | RP11-63L7.5; | | | | |
| | | | | (PRR2) | | | | |
| PIGB | HSG00290986 | 10025908827 | phosphatidylinositol | MGC21236 | 0.558 | 0.049 | −0.414 | 0.034 |
| | | | glycan | | | | | |
| | | | anchor | | | | | |
| | | | biosynthesis, | | | | | |
| | | | class B | | | | | |
| C14orf139 | HSG00287849 | 10023833484 | chromosome 14 | FLJ21276 | 0.557 | 0.049 | −0.444 | 0.023 |
| | | | open reading | | | | | |
| | | | frame 139 | | | | | |

1-73. (canceled)

74. A method for predicting the response of a patient diagnosed with a Notch mediated T-ALL to treatment with a Notch inhibitor comprising quantifying the gene expression level of a biomarker gene set designated Notch-10 comprising HES1, HES4, HES5, HESL, HEY-2, DTX1, MYC, NRARP, PTCRA and SHQ1 in a biological sample comprising cancer cells obtained from said subject to obtain a mean average expression level, wherein mean average expression level above or below a pre-determined cut-off level is predictive of the patient's treatment response to the anti-cancer agent.

75. The method according to claim 74, an increase in the average gene expression level relative to a pre-determined cut-off level is predictive of the patient's treatment response to the anti-cancer agent.

76. The method according to claim 74, wherein the predictive biomarker gene set comprises HES5, and wherein the average expression level of said gene set above or below a pre-determined cut-off level is predictive of the patient's treatment response to the anti-cancer agent.

77. The method according to claim 74, wherein the predictive biomarker gene set comprises DTX1, and wherein the average expression level of said gene set above or below a pre-determined cut-off level is predictive of the patient's treatment response to the anti-cancer agent.

78. The method according to claim 74, wherein the predictive biomarker gene set comprises HES1 and MYC, wherein the average expression level of said gene set above or below a pre-determined cut-off level is predictive of the patient's treatment response to the anti-cancer agent.

79. The method according to claim 74, wherein the predictive biomarker gene set comprises HES1 and HES5, wherein the average expression level of said gene set above or below a pre-determined cut-off level is predictive of the patient's treatment response to the anti-cancer agent.

80. The method according to any one of claim 74, wherein said patient is more likely than not to respond favorably to treatment with said Notch inhibitor when the expression levels of said biomarker gene in said clinical sample is increased relative to the cut-off level.

81. The method according to claim 80, wherein said patient is predicted to be sensitive to treatment with said Notch inhibitor.

82. The method according to any one of claim 74 wherein the pre-determined cut-off level has at least a statistically significant p-value over-expression in the biological sample comprising cancer cells relative to cells or tissue from normal patient or a patient not exhibiting aberrant Notch signaling.

83. The method according to any one of claim 74 wherein the pre-determined cut-off levels are at least 1 to 2 fold over-expressed in the biological sample relative to cells or tissue from a non-cancerous patient or cells or tissue comprising non-cancerous cells.

84. The method according to claim 83, wherein the p-value is less than 0.05.

85. The method according to claim 74 wherein gene expression is measured on a microarray or gene chip.

86. The method according to claim 85 wherein the microarray is a cDNA array or an oligonucleotide array.

87. The method according to claim 86 wherein the microarray or gene chip further comprises one or more internal control reagents.

88. The method to claim 74 wherein gene expression is determined by nucleic acid amplification conducted by polymerase chain reaction (PCR) of RNA extracted from the sample.

89. The method according to claim 74, wherein gene expression is detected by measuring or detecting a polypeptide encoded by the gene.

90. The method according to claim 89, wherein the polypeptide is detected by performing immunohistochemical analysis on the sample using an antibody that specifically binds to the polypeptide.

91. The method according to claim 74 wherein gene expression is detected by measuring a characteristic of the gene.

92. The method according to claim 74 wherein gene expression level is compared to an expression level of a control sample.

93. The method according to claim 74 wherein the determining step comprises detecting the RNA transcript levels.

94. The method according to claim 74, wherein said control is the average level of expression of each of said plurality of genes in a sample obtained from a disease free subject or a subject whose cells do not exhibit aberrant Notch signaling.

95. The method according to claim 74, wherein said average is the average level of expression of each of said plurality of genes across a plurality of control samples derived from disease free subjects.

96. A method for stratifying a patient diagnosed with a Notch mediated cellular proliferative disorder (T-ALL) for a clinical trial comprising:
(a) detecting a measured level of expression of a Notch biomarker gene set one in a clinical sample of diseased cells comprising cancer cells obtained from said subject with a control sample, and
(b) stratifying the patient for the clinical trial based on the results of the detecting step, wherein said gene set comprises HES1, HES4, HES5, HESL, HEY-2, DTX1, MYC, NRARP, PTCRA and SHQ1.

97. The method according to claim 96, wherein said gene set comprises DTX1.

98. The method according to claim 96, wherein said gene set comprises HES1 and HES5.

99. The method according to claim 96, wherein said gene set comprises HES1.

100. The method according to claim 96, wherein said gene set comprises HES5.

101. The method according to claim 96, wherein said gene set comprises HES1 and MYC.

102. A method for determining whether a patient diagnosed with a Notch mediated cellular proliferative disorder (T-ALL) is likely to respond to a Notch inhibitor based therapy comprising the steps of:
(a) quantifying the average expression level of a plurality of genes in a clinical sample of diseased cells obtained from a patient diagnosed with a Notch mediated cancer, wherein said plurality is genes is selected from the group comprising Group 1, Group 2 or Group 3, and
(b) comparing the average expression level obtained from the clinical sample to that obtained from a control sample, wherein an increase in the average expression in the clinical sample relative to the control sample indicates that the patient is more likely to respond to treatment with a Notch inhibitor;
wherein Group 1 comprises HES1, HES4, HES5, HESL, HEY-2, DTX1, MYC, NRARP, PTCRA and SHQ1HES1 and MYC; Group 2 comprises HES and HES5; Group 3 comprises HES1 and MYC.

103. The method according to claim 102, wherein said average level of expression in the clinical sample is the average level of expression of each of said plurality of genes.

104. The method according to claim 102, wherein said control is the average level of expression of each of said plurality of genes in a sample obtained from a disease free subject or a subject whose cells do not exhibit aberrant Notch signaling.

105. The method according to claim 102, wherein said average is the average level of expression of each of said plurality of genes across a plurality of control samples derived from disease free subjects.

106. A method of predicting the response of a patient diagnosed with a Notch mediated cellular proliferative disorder (T-ALL) to a Notch inhibitor, comprising: determining in a biological sample comprising cancer cells obtained from a patient after administration of a therapeutically effective amount of said Notch inhibitor the gene expression level of at least one target gene selected from the group consisting of HES4, HES5, DTX1, MYC, and SHQ1; wherein a change in the gene expression level of said at least one target gene relative to a control correlates with treatment response.

107. The method according to claim 106, wherein an increase in said mean average gene expression level of said at least one target gene in said biological sample obtained from said patient relative to a control sample correlates with poor prognosis of treatment response with said Notch inhibitor.

108. A method of predicting the response of a patient diagnosed with a Notch mediated cellular proliferative disorder (T-ALL) to a Notch inhibitor, comprising: determining in a biological sample comprising cancer cells obtained from a patient after administration of a therapeutically effective amount of said Notch inhibitor the gene expression level of at least one target gene selected from the group consisting of HES4, MYC, and SHQ1; wherein a change in the gene expression level of said at least one target gene relative to a control correlates with treatment response.

109. The method according to claim 108, wherein an increase in said mean average gene expression level of said at least one target gene in said biological sample obtained from said patient relative to a control sample correlates with poor prognosis of treatment response with said Notch inhibitor.

110. A method of predicting the response of a patient diagnosed with a Notch mediated cellular proliferative disorder (T-ALL) to a Notch inhibitor, comprising determining in a biological sample comprising cancer cells obtained from a patient after administration of a therapeutically effective amount of said Notch inhibitor the average gene expression level of a gene set comprising a plurality of genes selected from the group consisting of HES1, HES4, HES5, HESL, HEY-2, DTX1, MYC, NRARP, PTCRA and SHQ1 such as to obtain a mean average gene expression level, wherein a mean average gene expression level above or below a pre-determined cut-off level correlates with treatment response.

111. The method according to claim 110, wherein the determining step is carried out by a method comprising comparing the mean average level of gene expression of said plurality of genes in said set to the mean average gene expression level of the same genes in a control sample.

112. The method according to claim 110, wherein said control is the average level of expression of each of said plurality of genes in a sample of non-diseased cells or cells that do not exhibit aberrant Notch signaling or activation.

113. The method according to claim 110, wherein an increase in said mean average gene expression level of said plurality of genes in said gene set relative to a control sample correlates with poor prognosis of treatment response with said Notch inhibitor.

114. The method according to claim 110, wherein a decrease in said mean average gene expression level of said plurality of genes in said gene set relative to a control sample correlates with a good prognosis of treatment response with said Notch inhibitor.

115. A method of predicting the response of a patient diagnosed with a Notch mediated cellular proliferative disorder (T-ALL) to a Notch inhibitor, comprises determining in a biological sample comprising cancer cells obtained from a patient after administration of a therapeutically effective amount of said Notch inhibitor the gene expression level of at least one target gene selected from the group consisting of p19, p21 and p27, wherein a change in gene expression level of said at least one target gene above or below a pre-determined cut-off level correlates with treatment response.

116. The method according to claim 115, wherein the determining step is carried out by a method comprising comparing the gene expression of level of at least one of said target gene to the corresponding gene expression level in a control sample.

117. The method according to claim 115, wherein said control is the gene expression level determined in a control sample of non-diseased cells, or cells that do not exhibit aberrant Notch signaling or activation.

118. The method according to claim 115, wherein an increase in gene expression level of said at least one target gene in said biological sample obtained from said patient relative to a control sample correlates with good prognosis of treatment response with said Notch inhibitor.

119. A method to determine whether a patient diagnosed with a Notch mediated cancer (T-ALL) should continue treatment with a Notch inhibitor, comprising the steps of:
(a) determining the level of expression of at least one gene selected from the group consisting of p19, p21 and p27 in a clinical sample of cancer cells obtained from said patient prior to administering a therapeutically effective amount of a Notch inhibitor to said patient to obtain a pre-dosing level and after administration of said Notch inhibitor to obtain a post-dose level, and
(b) comparing said pre-dose and post-dose levels in said sample, wherein an increase in the expression level at least one biomarker gene selected from the group consisting of p19, p21 and p27 post-dose is predictive of said patient having a favorable response to treatment with said Notch inhibitor, whereas a decrease in the expression pattern of said at least one biomarker gene post-dose is predictive of an unfavorable response to treatment with said Notch inhibitor.

120. A method to determine whether a patient diagnosed with a Notch mediated cancer (T-ALL) should continue treatment with a Notch inhibitor, comprising determining the level of expression of at least one gene selected from the group consisting of HES4, HES5, DTX1, MYC and SHQ1 in a clinical sample of cancer cells obtained from said patient prior to administering a therapeutically effective amount of a Notch inhibitor to said patient to obtain a pre-dosing level and after administration of said Notch inhibitor to obtain a post-dose level, wherein a decrease in the expression level of said at least one biomarker gene post-dose is predictive of said patient having a favorable response to treatment with said Notch inhibitor.

121. A method to determine whether a patient diagnosed with a Notch mediated cancer (T-ALL) should continue treatment with a Notch inhibitor, comprising the steps of:
(a) determining the average level of expression of each of a plurality of biomarker gene selected from the group consisting of HES1, HES4, HES5, HESL, HEY-2, MYC, NRARP, PTCRA, DTX1 in a clinical sample of cancer cells obtained from said patient aid prior to (pre-dose) and after (post-dose) administration of a therapeutically effective amount of a Notch inhibitor to said patient to obtain a pre-dosing level, and
(b) quantifying said level of expression in each of the pre- and post-dose samples to obtain an average mean level of expression, wherein a decrease in the average level of expression in the post-dose sample relative to the pre-dose sample is predictive of said patient having a favorable response to treatment with said Notch inhibitor, whereas an increase in the average mean expression level in said post-dose sample relative to the pre-dose sample is predictive of an unfavorable response to treatment with said Notch inhibitor.

122. A method for determining the therapeutic efficacy of a Notch inhibitor for treating a Notch mediated cellular proliferative disorder (T-ALL) comprising assaying a sample of diseased cells from said subject to determine and quantify a mean average expression level of each of a plurality of genes selected from the group consisting of HES1, HES4, HES5, HESL, HEY-2, DTX1, MYC, NRARP, PTCRA, SHQ1 at a first time point after administration of a therapeutically effective amount of said Notch inhibitor, wherein a decrease in the mean average level of expression of said plurality if genes relative to a control sample is indicative of the therapeutic efficacy of said Notch inhibitor.

123. The method according to claim 122, further comprising the step of assaying a sample of diseased cells post administration over at least one additional time point, wherein a decrease in the mean average expression level is indicative of the therapeutic efficacy of said inhibitor.

124. A method for determining the therapeutic efficacy of a Notch inhibitor for treating a Notch mediated cellular proliferative disorder (T-ALL) comprising the step of assaying a sample of diseased cells for expression levels of at least one biomarker gene selected from the group consisting of p19, p21 and p27 post administration of said Notch inhibitor over a period of time, wherein an increase in the level of expression of said at least one biomarker gene over a period of time relative to a control sample is indicative of the therapeutic efficacy of said Notch inhibitor.

125. A method of determining a therapeutically effective dosage of a Notch inhibitor to effectively treat a Notch mediated cellular proliferative disorder (T-ALL) in a subject comprising the steps of:
(a) administering to a diseased non-human animal varying dosages of said Notch inhibitor,
(b) determining in a biological sample obtained from said subject after administration of each dosage a gene expression profile of at least one biomarker gene selected from the group consisting of HES1, HES4, HES5, HESL, HEY-2, DTX1, MYC, NRARP, PTCRA, SHQ1, p19, p21, and p27; and
(c) selecting an appropriate dosage based upon the results of the gene expression profile.

126. The method according to claim 125, wherein determining gene expression profiles comprises determining the gene expression level of at least one said biomarker gene across a plurality of biological samples relative to a control or a pre-determined cut-off level.

127. The method according to claim 125, wherein the appropriate dosage correlates with gene expression levels above or below a pre-determined cut-off level.

128. The method according to claim 125, wherein the step of selecting the appropriate dosage comprises selecting a dosage regiment that results in a statistically significant decrease or increase in the gene expression level of said at least one gene relative to a control.
